(19) United States
(12) Patent Application Publication
(10) Pub. No.: US 2024/0294533 A1
(43) Pub. Date: Sep. 5, 2024

(54) 2-(HET)ARYL-SUBSTITUTED CONDENSED HETEROCYCLE DERIVATIVES AS PEST CONTROL AGENTS

(71) Applicant: Bayer Aktiengesellschaft, Leverkusen (DE)

(72) Inventors: Ruediger FISCHER, Pulheim (DE); Steffen MUELLER, Muelheim an der Ruhr (DE); Matthieu WILLOT, Monheim am Rhein (DE); Elke HELLWEGE, Berlin (DE); Marc LINKA, Duesseldorf (DE); Peter LOESEL, Leverkusen (DE); Nina KAUSCH-BUSIES, Dusseldorf (DE); Yolanda CANCHO GRANDE, Leverkusen (DE); Yeshua SEMPERE MOLINA, Langenfeld (DE); Sascha EILMUS, Leichlingen (DE); Kerstin ILG, Koeln (DE); Ulrich GOERGENS, Ratingen (DE)

(21) Appl. No.: 18/559,710

(22) PCT Filed: May 10, 2022
(86) PCT No.: PCT/EP2022/062620

§ 371 Date: Nov. 8, 2023

(30) Foreign Application Priority Data
May 12, 2021 (EP) 21173484.3

(51) Int. Cl. C07D 487/04; A01N 25/04; A01N 43/90; A01P 7/04; C07D 471/04

(52) U.S. Cl. CPC C07D 487/04; A01N 25/04; A01N 43/90; A01P 7/04; C07D 471/04

(57) ABSTRACT

The invention relates to novel compounds of the formula (I)

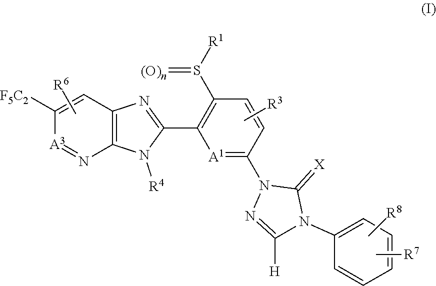

in which A¹, A³, X, R¹, R³, R⁴, R⁶, R⁷, R⁸ and n have the definitions given above,
to the use thereof as acaricides and/or insecticides for controlling animal pests and to processes and intermediates for preparation thereof.

The present invention relates to novel 2-(het)aryl-substituted fused heterocycle derivatives of the formula (I), to the use thereof as acaricides and/or insecticides for controlling animal pests, particularly arthropods and especially insects and arachnids, and to processes and intermediates for preparation thereof.

Fused heterocycle derivatives with insecticidal properties are already described in the literature, e.g. in WO 2010/125985, WO 2012/074135, WO 2012/086848, WO 2013/018928, WO 2013/191113, WO 2014/142292, WO 2014/148451, WO 2015/000715, WO 2016/124563, WO 2016/124557, WO 2015/121136, WO 2015/133603, WO 2015/198859, WO 2015/002211, WO 2015/071180, WO 2015/091945, WO 2016/005263, WO 2015/198817, WO 2016/041819, WO 2016/039441, WO 2016/026848, WO 2016/023954, WO 2016/020286, WO 2016/046071, WO 2017/025419, WO 2017/055185, WO 2017/121674, WO 2018/141954 or WO 2021/213978.

Modern crop protection compositions have to meet many demands, for example in relation to extent, persistence and spectrum of their action and possible use. Questions of toxicity, sparing of beneficial species and pollinators, environmental properties, application rates, combinability with other active ingredients or formulation auxiliaries play a role, as does the question of the effort required for the synthesis of an active ingredient; furthermore, resistances may occur, to mention just some parameters. For all these reasons alone, the search for novel crop protection compositions cannot be considered complete, and there is a constant need for novel compounds having improved properties compared to the known compounds, at least in relation to individual aspects.

It was an object of the present invention to provide compounds which broaden the spectrum of the pesticides in various aspects and/or improve their activity.

Novel 2-(het)aryl-substituted fused heterocycle derivatives have now been found, and these have advantages over the compounds already known, examples of which are better biological or environmental properties, a wider range of application methods, better insecticidal or acaricidal activity, and also good compatibility with crop plants. The 2-(het)aryl-substituted fused heterocycle derivatives can be used in combination with further agents for improving efficacy, especially against insects that are difficult to control.

The present invention therefore provides novel compounds of the formula (I)

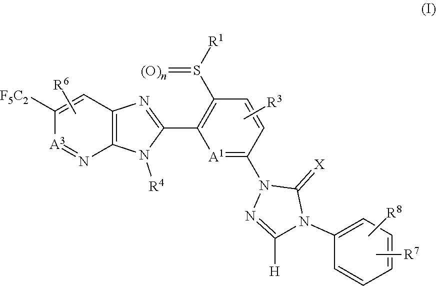

in which (configuration 1-1)
A¹ is nitrogen, =N+(O⁻)— or =C(H)—,
A³ is nitrogen, =N+(O⁻)— or =C(H)—,
X is oxygen or sulfur,
R¹ is (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₈)cycloalkyl, halo(C₃-C₈)cycloalkyl, (C₃-C₈)cycloalkyl-(C₁-C₆)alkyl, (C₃-C₈)cycloalkyl-(C₁-C₆)haloalkyl, (C₁-C₆)alkyl-(C₃-C₈)cycloalkyl, (C₁-C₆)haloalkyl-(C₃-C₈)cycloalkyl, (C₁-C₆)cyanoalkyl, (C₁-C₆)alkoxy-(C₁-C₆)alkyl, (C₁-C₆)haloalkoxy-(C₁-C₆)alkyl, (C₁-C₆)alkylthio-(C₁-C₆)alkyl, (C₁-C₆)alkylsulfinyl-(C₁-C₆)alkyl or (C₁-C₆)alkylsulfonyl-(C₁-C₆)alkyl,
R³ is hydrogen, cyano, halogen, nitro, hydroxyl, amino, SCN, tri-(C₁-C₆)alkylsilyl, (C₃-C₈)cycloalkyl, (C₃-C₈)cycloalkyl-(C₃-C₈)cycloalkyl, (C₁-C₆)alkyl-(C₃-C₈)cycloalkyl, halo(C₃-C₈)cycloalkyl, cyano(C₃-C₈)cycloalkyl, (C₁-C₆)alkyl, (C₁-C₆)haloalkyl, (C₁-C₆)cyanoalkyl, (C₁-C₆)hydroxyalkyl, (C₁-C₆)alkoxy-(C₁-C₆)alkyl, (C₂-C₆)alkenyl, (C₂-C₆)haloalkenyl, (C₂-C₆)cyanoalkenyl, (C₂-C₆)alkynyl, (C₂-C₆)haloalkynyl, (C₂-C₆)cyanoalkynyl, (C₁-C₆)alkoxy, (C₁-C₆)haloalkoxy, (C₁-C₆)cyanoalkoxy, (C₁—C₆)alkylhydroxyimino, (C₁-C₆)alkoxyimino, (C₁-C₅)alkyl-(C₁-C₆)alkoxyimino, (C₁-C₆)haloalkyl-(C₁-C₆)alkoxyimino, (C₁-C₆)alkylthio, (C₁-C₆)haloalkylthio, (C₁-C₆)alkylsulfinyl, (C₁-C₆)haloalkylsulfinyl, (C₁-C₆)alkylsulfonyl, (C₁-C₆)haloalkylsulfonyl, (C₁-C₆)alkylcarbonyl, (C₁-C₆)haloalkylcarbonyl, (C₁-C₆)alkoxycarbonyl, (C₁-C₆)haloalkoxycarbonyl, aminocarbonyl, (C₁-C₆)alkylaminocarbonyl, di(C₁-C₆)alkylaminocarbonyl, (C₁-C₆)alkylsulfonylamino, (C₁-C₆)alkylamino, di(C₁-C₆)alkylamino, aminosulfonyl, (C₁-C₆)alkylaminosulfonyl, di(C₁-C₆)alkylaminosulfonyl, (C₁-C₆)alkylsulfoximino, (C₃-C₅)cycloalkylamino or NHCO—(C₁-C₆)alkyl ((C₁-C₆)alkylcarbonylamino),
R⁴ is hydrogen, (C₁-C₄)alkyl, (C₁-C₄)haloalkyl, (C₁-C₄)cyanoalkyl, (C₁-C₄)alkoxy-(C₁-C₄)alkyl, (C₂-C₄)alkenyl, (C₂-C₄)haloalkenyl, (C₂-C₄)cyanoalkenyl, (C₂-C₄)alkynyl, (C₂-C₄)haloalkynyl, (C₂-C₄)cyanoalkynyl, (C₁-C₄)alkoxy, (C₁-C₄)haloalkoxy, (C₁-C₄)alkylthio, (C₁-C₄)haloalkylthio, (C₁-C₄)alkylsulfinyl, (C₁-C₄)haloalkylsulfinyl, (C₁-C₄)alkylsulfonyl or (C₁-C₄)haloalkylsulfonyl,
R⁶ is hydrogen, cyano, halogen, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₅)cycloalkyl, (C₃-C₅)cycloalkyl-(C₃-C₅)cycloalkyl, (C₁-C₆)alkyl-(C₃-C₅)cycloalkyl, (C₁-C₆)alkoxy, (C₁-C₆)haloalkoxy, (C₁-C₆)alkoxyimino, (C₁-C₆)alkylthio, (C₁-C₆)haloalkylthio, (C₁-C₆)alkylsulfinyl, (C₁-C₆)haloalkylsulfinyl, (C₁-C₆)alkylsulfonyl, (C₁-C₆)haloalkylsulfonyl, (C₁-C₆)alkylsulfonyloxy, (C₁-C₆)alkylcarbonyl, (C₁-C₆)haloalkylcarbonyl, aminocarbonyl, (C₁-C₆)alkylaminocarbonyl, di(C₁-C₆)alkylaminocarbonyl, (C₁-C₆)alkylsulfonylamino, aminosulfonyl, (C₁-C₆)alkylaminosulfonyl or di(C₁-C₆)alkylaminosulfonyl,
R⁷, R⁸ are independently hydrogen, cyano, halogen, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₈)cycloalkyl, halo(C₃-C₈)cycloalkyl, cyano(C₃-C₈)cycloalkyl, (C₃-C₈)cycloalkyl-(C₃-C₈)cycloalkyl, (C₁-C₆)alkyl-(C₃-C₈)cycloalkyl, (C₁-C₆)alkoxy, (C₁-C₆)haloalkoxy, (C₁-C₆)alkoxyimino, (C₁-C₆)alkylthio, (C₁-C₆)haloalkylthio, (C₁-C₆)alkylsulfinyl, (C₁-C₆)haloalkylsulfinyl, (C₁—C₆)alkylsulfonyl, (C₁-C₆)haloalkylsulfonyl, (C₁-C₆)alkylcarbonyl, (C₁-C₆)haloalkylcarbonyl, (C₁-C₆)alkoxycarbonyl, aminocarbonyl, (C₁-C₆)alkylaminocarbonyl, di(C₁-C₆)alkylaminocarbonyl, (C₁-C₆)alkylsulfonylamino, aminosulfonyl, (C₁-C₆)alkylaminosulfonyl or di(C₁-C₆)alkylaminosulfonyl,
n is 0, 1 or 2.

It has additionally been found that the compounds of the formula (I) have very good efficacy as pesticides, preferably as insecticides and/or acaricides, and additionally generally have very good plant compatibility, in particular with respect to crop plants.

The compounds of the invention are defined in general terms by the formula (I). Preferred substituents or ranges of the radicals given in the formulae mentioned above and below are illustrated hereinafter:

### Configuration 2-1

A¹ is preferably nitrogen, =N⁺(O⁻)— or =C(H)—,
A³ is preferably nitrogen, =N⁺(O⁻)— or =C(H)—,
X is preferably oxygen or sulfur,
R¹ is preferably (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₈)cycloalkyl, halo(C₃-C₈)cycloalkyl, (C₃-C₈)cycloalkyl-(C₁-C₆)alkyl, (C₃-C₈)cycloalkyl-(C₁-C₆)haloalkyl, (C₁-C₆)alkyl-(C₃-C₈)cycloalkyl, (C₁-C₆)haloalkyl-(C₃-C₈)cycloalkyl, (C₁-C₆)cyanoalkyl, (C₁-C₆)alkoxy-(C₁-C₆)alkyl or (C₁-C₆)haloalkoxy-(C₁-C₆)alkyl,
R³ is preferably hydrogen, cyano, halogen, (C₃-C₈)cycloalkyl, (C₁-C₆)alkyl-(C₃-C₈)cycloalkyl, halo(C₃-C₈)cycloalkyl, cyano(C₃-C₈)cycloalkyl, (C₁-C₆)alkyl, (C₁-C₆)haloalkyl, (C₁-C₆)cyanoalkyl, (C₁-C₆)alkoxy-(C₁-C₆)alkyl, (C₂-C₆)alkenyl, (C₂-C₆)haloalkenyl, (C₂-C₆)cyanoalkenyl, (C₂-C₆)alkynyl, (C₂-C₆)haloalkynyl, (C₂-C₆)cyanoalkynyl, (C₁-C₆)alkoxy, (C₁-C₆)haloalkoxy, (C₁-C₆)cyanoalkoxy, (C₁-C₆)alkylhydroxyimino, (C₁-C₆)alkoxyimino, (C₁-C₆)alkyl-(C₁-C₆)alkoxyimino, (C₁-C₆)haloalkyl-(C₁-C₆)alkoxyimino, (C₁—C₆)alkylthio, (C₁-C₆)haloalkylthio, (C₁-C₆)alkylsulfinyl, (C₁-C₆)haloalkylsulfinyl, (C₁-C₆)alkylsulfonyl, (C₁-C₆)haloalkylsulfonyl, aminocarbonyl, (C₁-C₆)alkylaminocarbonyl, di(C₁-C₆)alkylaminocarbonyl, (C₁-C₆)alkylsulfonylamino, aminosulfonyl, (C₁-C₆)alkylaminosulfonyl, di(C₁-C₆)alkylaminosulfonyl, (C₁-C₆)alkylsulfoximino or NHCO—(C₁-C₆)alkyl ((C₁-C₆)alkylcarbonylamino),
R⁴ is preferably hydrogen, (C₁-C₄)alkyl, (C₁-C₄)alkoxy-(C₁-C₄)alkyl, (C₁-C₄)haloalkyl, (C₂-C₄)alkenyl, (C₂-C₄)haloalkenyl, (C₂-C₄)alkynyl, (C₂-C₄)haloalkynyl, (C₁-C₄)alkoxy, (C₁-C₄)haloalkoxy, (C₁-C₄)alkylthio, (C₁-C₄)haloalkylthio, (C₁-C₄)alkylsulfinyl, (C₁-C₄)haloalkylsulfinyl, (C₁-C₄)alkylsulfonyl or (C₁-C₄)haloalkylsulfonyl,
R⁶ is preferably hydrogen, cyano, halogen, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₈)cycloalkyl, (C₁-C₆)alkyl-(C₃-C₈)cycloalkyl, (C₁-C₆)alkoxy, (C₁-C₆)haloalkoxy, (C₁-C₆)alkoxyimino, (C₁-C₆)alkylthio, (C₁-C₆)haloalkylthio, (C₁-C₆)alkylsulfinyl, (C₁-C₆)haloalkylsulfinyl, (C₁-C₆)alkylsulfonyl, (C₁-C₆)haloalkylsulfonyl, (C₁-C₆)alkylcarbonyl or (C₁-C₆)haloalkylcarbonyl,
R⁷, R⁸ are preferably independently hydrogen, cyano, halogen, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₈)cycloalkyl, halo(C₃-C₈)cycloalkyl, cyano(C₃-C₈)cycloalkyl, (C₃-C₈)cycloalkyl-(C₃-C₈)cycloalkyl, (C₁-C₆)alkyl-(C₃-C₈)cycloalkyl, (C₁-C₆)alkoxy, (C₁-C₆)haloalkoxy, (C₁-C₆)alkoxycarbonyl, (C₁-C₆)alkoxyimino, (C₁-C₆)alkylthio, (C₁-C₆)haloalkylthio, (C₁-C₆)alkylsulfinyl, (C₁-C₆)haloalkylsulfinyl, (C₁-C₆)alkylsulfonyl or (C₁-C₆)haloalkylsulfonyl,
n is preferably 0, 1 or 2.

### Configuration 3-1

A¹ is more preferably nitrogen, =N⁺(O⁻)— or =C(H)—,
A³ is more preferably nitrogen, =N⁺(O⁻)— or =C(H)—,
X is more preferably oxygen or sulfur,
R¹ is more preferably (C₁-C₆)alkyl, (C₁-C₆)haloalkyl or (C₃-C₆)cycloalkyl,
R³ is more preferably hydrogen, cyano, halogen, (C₃-C₆)cycloalkyl, (C₁-C₆)alkyl-(C₃-C₆)cycloalkyl, halo(C₃-C₆)cycloalkyl, cyano(C₃-C₆)cycloalkyl, (C₁-C₆)alkyl, (C₁-C₆)haloalkyl, (C₁-C₆)cyanoalkyl, (C₁-C₆)alkoxy-(C₁-C₆)alkyl, (C₂-C₆)alkenyl, (C₂-C₆)haloalkenyl, (C₂-C₆)cyanoalkenyl, (C₂-C₆)alkynyl, (C₂-C₆)haloalkynyl, (C₂-C₆)cyanoalkynyl, (C₁-C₆)alkoxy, (C₁-C₆)haloalkoxy, (C₁-C₆)alkoxyimino, (C₁-C₆)alkylthio, (C₁-C₆)haloalkylthio, (C₁-C₆)alkylsulfinyl, (C₁-C₆)haloalkylsulfinyl, (C₁-C₆)alkylsulfonyl, (C₁-C₆)haloalkylsulfonyl or (C₁-C₆)alkylsulfoximino,
R⁴ is more preferably hydrogen, (C₁-C₄)alkyl, (C₁-C₄), alkoxy-(C₁-C₄)alkyl or (C₁-C₄)haloalkyl,
R⁶ is more preferably hydrogen, cyano, halogen, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₆)cycloalkyl, (C₁-C₆)alkoxy, (C₁-C₆)haloalkoxy, (C₁-C₆)alkylthio, (C₁-C₆)haloalkylthio, (C₁-C₆)alkylsulfinyl, (C₁-C₆)haloalkylsulfinyl, (C₁-C₆)alkylsulfonyl, (C₁-C₆)haloalkylsulfonyl, (C₁-C₆)alkylcarbonyl or (C₁-C₆)haloalkylcarbonyl,
R⁷, R⁸ are more preferably independently hydrogen, cyano, halogen, (C₁-C₄)alkyl, (C₁-C₄)haloalkyl, (C₃-C₆)cycloalkyl, halo(C₃-C₆)cycloalkyl, cyano(C₃-C₆)cycloalkyl, (C₁-C₄)alkoxy, (C₁-C₄)haloalkoxy, (C₁-C₄)alkoxycarbonyl, (C₁-C₄)alkoxyimino, (C₁-C₄)alkylthio, (C₁-C₄)haloalkylthio, (C₁-C₄)alkylsulfinyl, (C₁-C₄)haloalkylsulfinyl, (C₁-C₄)alkylsulfonyl or (C₁-C₄)haloalkylsulfonyl,
n is more preferably 0, 1 or 2.

### Configuration 4-1

A¹ is even more preferably nitrogen,
A³ is even more preferably nitrogen or =C(H)—,
X is even more preferably oxygen,
R¹ is even more preferably (C₁-C₄)alkyl, (C₁-C₄)haloalkyl or (C₃-C₄)cycloalkyl,
R³ is even more preferably hydrogen, cyano, halogen, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, (C₁-C₄)alkylsulfinyl, (C₁-C₄)haloalkylsulfinyl, (C₁-C₄)alkylsulfonyl, (C₁-C₄)haloalkylsulfonyl or (C₁-C₄)alkoxyimino,
R⁴ is even more preferably hydrogen or (C₁-C₄)alkyl,
R⁶ is even more preferably hydrogen,
R⁷ is even more preferably cyano, halogen, (C₁-C₄)alkyl, (C₁-C₄)haloalkyl, (C₃-C₄)cycloalkyl, cyano(C₃-C₄)cycloalkyl, (C₁-C₄)alkoxy, (C₁-C₄)haloalkoxy, (C₁-C₄)alkoxycarbonyl, (C₁-C₄)alkoxyimino, (C₁-C₄)alkylthio, (C₁-C₄)haloalkylthio, (C₁-C₄)alkylsulfinyl, (C₁-C₄)haloalkylsulfinyl, (C₁-C₄)alkylsulfonyl or (C₁-C₄)haloalkylsulfonyl,
R⁸ is even more preferably hydrogen or cyano,
n is even more preferably 0, 1 or 2.

### Configuration 5-1

A¹ is particularly nitrogen,
A³ is particularly nitrogen or =C(H)—,
X is particularly oxygen,
R¹ is particularly methyl, ethyl, n-propyl or i-propyl,
R³ is particularly hydrogen,
R⁴ is particularly methyl,
R⁶ is particularly hydrogen,
R⁷ is particularly cyano, fluorine, chlorine, bromine, iodine, methyl, ethyl, trifluoromethyl, methoxy, trifluoromethoxy, methoxycarbonyl, methoxyimino or cyanocyclopropyl,
R⁸ is particularly hydrogen or chlorine,
n is particularly 0, 1 or 2.

### Configuration 6-1

A¹ is especially nitrogen,
A³ is especially nitrogen or =C(H)—,
X is especially oxygen,
R¹ is especially ethyl,
R³ is especially hydrogen,
R⁴ is especially methyl,
R⁶ is especially hydrogen,
R⁷ is especially cyano, fluorine, chlorine, bromine, iodine, methoxycarbonyl (—COOCH₃), methoxyimino (—CH═NOCH₃) or 1-cyano-1-cyclopropyl,
R⁸ is especially hydrogen or chlorine,
n is especially 2.

In a preferred embodiment, the invention relates to compounds of the formula (I) where A¹ is nitrogen and A³, X, R¹, R³, R⁴, R⁶, R⁷, R⁸ and n have the definitions given in configuration (1-1) or configuration (2-1) or configuration (3-1) or configuration (4-1) or configuration (5-1) or configuration (6-1).

In a preferred embodiment, the invention relates to compounds of the formula (I) where A¹ is nitrogen, A³ is nitrogen, and X, R¹, R³, R⁴, R⁶, R⁷, R⁸ and n have the definitions given in configuration (1-1) or configuration (2-1) or configuration (3-1) or configuration (4-1) or configuration (5-1) or configuration (6-1).

In a preferred embodiment, the invention relates to compounds of the formula (I) where A¹ is nitrogen, A³ is =C(H)—, and X, R¹, R³, R⁴, R⁶, R⁷, R⁸ and n have the definitions given in configuration (1-1) or configuration (2-1) or configuration (3-1) or configuration (4-1) or configuration (5-1) or configuration (6-1).

In a further embodiment, the invention relates to the compounds of the formula (I) where A¹ is nitrogen, A³ is nitrogen, R⁴ is methyl, X is oxygen, R³ is hydrogen, R⁶ is hydrogen, and R¹, R⁷, R⁸ and n have the definitions given in configuration (1-1) or configuration (2-1) or configuration (3-1) or configuration (4-1) or configuration (5-1) or configuration (6-1).

In a further embodiment, the invention relates to the compounds of the formula (I) where A¹ is nitrogen, A³ is =C(H)—, R⁴ is methyl, X is oxygen, R³ is hydrogen, R⁶ is hydrogen, and R¹, R⁷, R⁸ and n have the definitions given in configuration (1-1) or configuration (2-1) or configuration (3-1) or configuration (4-1) or configuration (5-1) or configuration (6-1).

In a further embodiment, the invention relates to the compounds of the formula (IA)

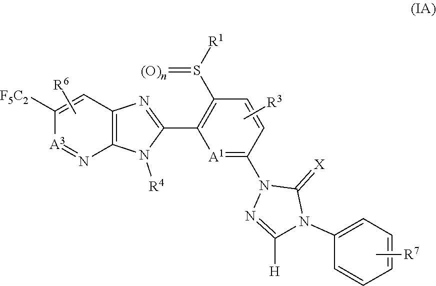

in which A¹, A³, X, R¹, R³, R⁴, R⁶, R⁷ and n have the definitions given in configuration (1-1) or configuration (2-1) or configuration (3-1) or configuration (4-1) or configuration (5-1) or configuration (6-1).

In a preferred embodiment, the invention relates to compounds of the formula (IA) where A¹ is nitrogen, A³ is nitrogen, and X, R¹, R³, R⁴, R⁶, R⁷ and n have the definitions given in configuration (1-1) or configuration (2-1) or configuration (3-1) or configuration (4-1) or configuration (5-1) or configuration (6-1).

In a preferred embodiment, the invention relates to compounds of the formula (IA) where A¹ is nitrogen, A³ is =C(H)—, and X, R¹, R³, R⁴, R⁶, R⁷ and n have the definitions given in configuration (1-1) or configuration (2-1) or configuration (3-1) or configuration (4-1) or configuration (5-1) or configuration (6-1).

In a further embodiment, the invention relates to the compounds of the formula (IA) where A¹ is nitrogen, A³ is nitrogen, R⁴ is methyl, X is oxygen, R³ is hydrogen, R⁶ is hydrogen, and R¹, R⁷ and n have the definitions given in configuration (1-1) or configuration (2-1) or configuration (3-1) or configuration (4-1) or configuration (5-1) or configuration (6-1).

In a further embodiment, the invention relates to the compounds of the formula (IA) where A¹ is nitrogen, A³ is =C(H)—, R⁴ is methyl, X is oxygen, R³ is hydrogen, R⁶ is hydrogen, and R¹, R⁷ and n have the definitions given in configuration (1-1) or configuration (2-1) or configuration (3-1) or configuration (4-1) or configuration (5-1) or configuration (6-1).

In the preferred definitions, unless stated otherwise, halogen is selected from the group of fluorine, chlorine, bromine and iodine, preferably in turn from the group of fluorine, chlorine and bromine.

In the particularly preferred definitions, unless stated otherwise, halogen is selected from the group of fluorine, chlorine, bromine and iodine, preferably in turn from the group of fluorine, chlorine and bromine.

In the context of the present invention, unless defined differently elsewhere, the term “alkyl”, either on its own or else in combination with further terms, for example haloalkyl, is understood to mean a radical of a saturated, aliphatic hydrocarbon group which has 1 to 12 carbon atoms and may be branched or unbranched. Examples of C₁-C₁₂-alkyl radicals are methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1-ethylpropyl, 1,2-dimethylpropyl, hexyl, n-heptyl, n-octyl, n-nonyl, n-decyl, n-undecyl and n-dodecyl. Among these alkyl radicals, particular preference is given to C₁-C₆-alkyl radicals. Particular preference is given to C₁-C₄-alkyl radicals.

According to the invention, unless defined differently elsewhere, the term “alkenyl”, either on its own or else in combination with further terms, is understood to mean a straight-chain or branched C₂-C₁₂-alkenyl radical which has at least one double bond, for example vinyl, allyl, 1-propenyl, isopropenyl, 1-butenyl, 2-butenyl, 3-butenyl, 1,3-butadienyl, 1-pentenyl, 2-pentenyl, 3-pentenyl, 4-pentenyl, 1,3-pentadienyl, 1-hexenyl, 2-hexenyl, 3-hexenyl, 4-hexenyl, 5-hexenyl and 1,4-hexadienyl. Among these, preference is given to C₂-C₆-alkenyl radicals and particular preference to C₂-C₄-alkenyl radicals.

According to the invention, unless defined differently elsewhere, the term “alkynyl”, either on its own or else in combination with further terms, is understood to mean a straight-chain or branched C₂-C₁₂-alkynyl radical which has at least one triple bond, for example ethynyl, 1-propynyl and propargyl. Among these, preference is given to C₃-C₆-alkynyl radicals and particular preference to C₃-C₄-alkynyl radicals. The alkynyl radical may also contain at least one double bond.

According to the invention, unless defined differently elsewhere, the term “cycloalkyl”, either on its own or else in combination with further terms, is understood to mean a C₃-C₈-cycloalkyl radical, for example cyclopropyl, cyclobutyl, cyclopentyl, cyclohexyl, cycloheptyl and cyclooctyl. Among these, preference is given to C₃-C₆-cycloalkyl radicals.

The term “alkoxy”, either on its own or else in combination with further terms, for example haloalkoxy, is understood in the present case to mean an O-alkyl radical, where the term “alkyl” is as defined above.

Halogen-substituted radicals, for example haloalkyl, are mono- or polyhalogenated up to the maximum number of possible substituents. In the case of polyhalogenation, the halogen atoms may be identical or different. In this case, halogen is fluorine, chlorine, bromine or iodine, especially fluorine, chlorine or bromine.

Unless stated otherwise, optionally substituted radicals may be mono- or polysubstituted, where the substituents in the case of polysubstitutions may be the same or different.

The radical definitions or illustrations given above in general terms or listed within ranges of preference apply correspondingly to end products and to starting materials and intermediates. These radical definitions can be combined with one another as desired, i.e. including combinations between the respective ranges of preference.

Preference is given in accordance with the invention to using compounds of the formula (I) in which there is a combination of the definitions listed above as being preferred.

Particular preference is given in accordance with the invention to using compounds of the formula (I) in which there is a combination of the definitions listed above as being more preferred.

Very particular preference is given in accordance with the invention to using compounds of the formula (I) in which there is a combination of the definitions listed above as being even more preferred.

Particularly used in accordance with the invention are compounds of the formula (I) in which there is a combination of the definitions listed above as particular definitions.

Especially used in accordance with the invention are compounds of the formula (I) in which there is a combination of the definitions listed above as especial definitions.

### Isomers

Depending on the nature of the substituents, the compounds of the formula (I) may be in the form of geometric and/or optically active isomers or corresponding isomer mixtures in different compositions. These stereoisomers are, for example, enantiomers, diastereomers, atropisomers or geometric isomers. The invention therefore encompasses both pure stereoisomers and any desired mixtures of these isomers.

### Isotope Variants

The present invention also includes all suitable isotope variants of the compounds of the formula (I). An isotope variant of such a compound should be considered to mean a compound of the formula (I) in which at least one atom is replaced by another atom having the same atomic number but an atomic mass different from that usually or predominantly encountered in nature. Examples of isotopes which can be incorporated into a compound of the formula (I) are those of hydrogen, carbon, nitrogen, oxygen, phosphorus, sulfur, fluorine, chlorine, bromine and iodine, such as ²H (deuterium), ³H (tritium), ¹³C, ¹⁴C, ¹⁵N, ¹⁷O ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotope variants of a compound of the formula (I), such as, in particular, those incorporated into the one or more radioactive isotopes, may be beneficial, for example, for studies of the mechanism of action or of the active ingredient distribution, for example in the body of a pathogen; especially suitable for this purpose are compounds labelled with ³H or ¹⁴C isotopes, since the preparation and detection thereof is comparatively simple. Furthermore, the incorporation of isotopes, for example deuterium, can offer advantages, for example on account of a greater metabolic stability of the compound, for example an extension of the half-life or a reduction in the required active dose. Isotope modifications of the compounds of the formula (I) may therefore also constitute a preferred embodiment of the invention. Isotope variants of the compounds of the formula (I) can be prepared by processes known to the person skilled in the art, for example by the processes described below and the instructions given in the illustrative embodiments, using corresponding isotope modifications of the respective reagents and/or starting compounds (reactants).

The inventive compounds of the formula (I) can be obtained by the process shown in the following scheme:

### Process a

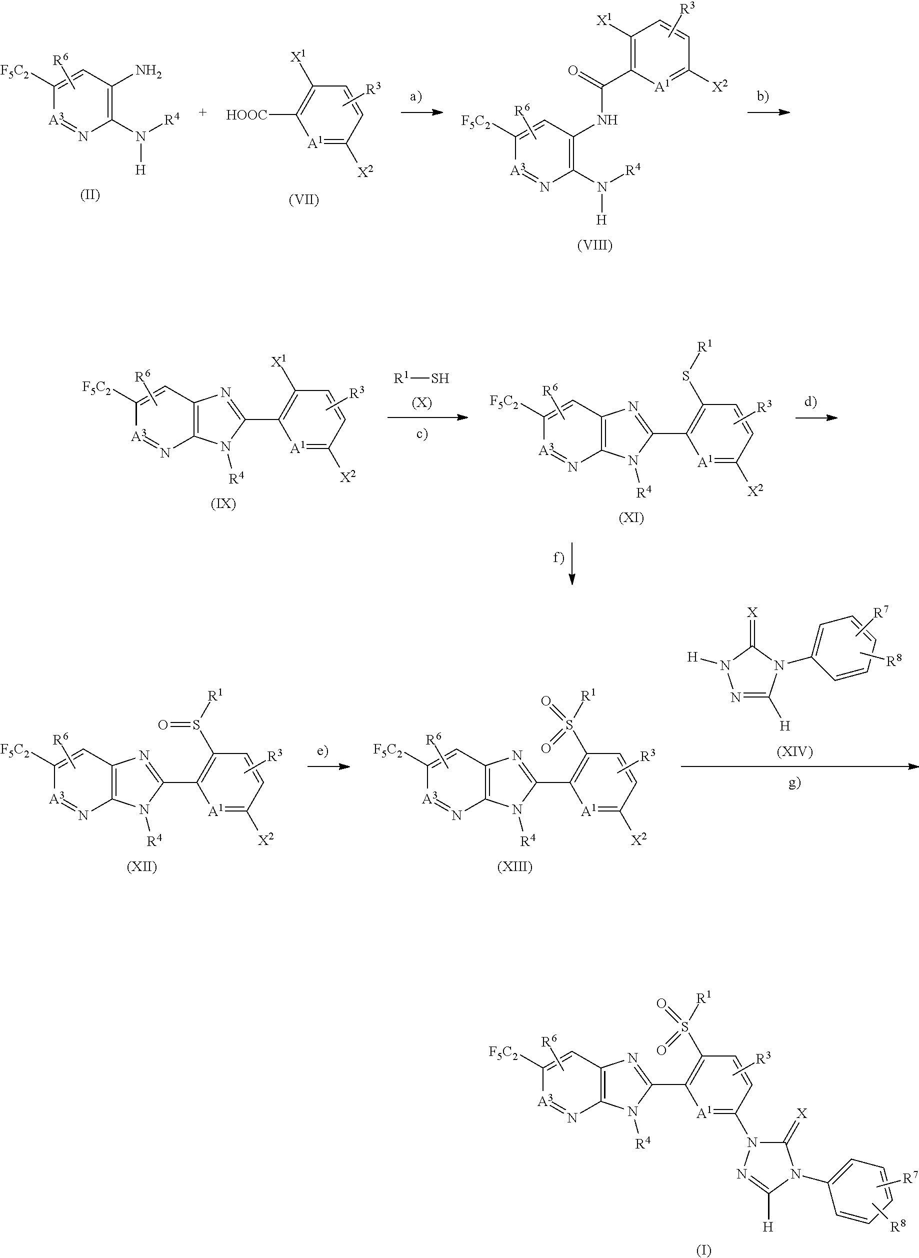

The R¹, R³, R⁴, R⁶, R⁷, R⁸, A¹, A³ and X radicals have the definitions described above; X¹ and X² are halogen.

### Step a)

The compounds of the formula (VIII) can be prepared in analogy to the process described in U.S. Pat. No. 5,576,335 by the reaction of compounds of the formula (II) with a carboxylic acid of the formula (VII) in the presence of a condensing agent or a base.

Compounds of the formula (II) are either commercially available or can be prepared by known methods, for example analogously to the processes described in WO2017/014214, WO2016/194929 or Journal of Medicinal Chemistry 62 (2019), 11232-11259.

Carboxylic acids of the formula (VII) are either commercially available or can be prepared by known methods, for example analogously to the processes described in US2010/234604, WO2012/61926 or Bioorganic and Medicinal Chemistry Letters, 18 (2008), 5023-5026.

The reaction of the compounds of the formula (II) with carboxylic acids of the formula (VII) can be effected neat or in a solvent, preference being given to conducting the reaction in a solvent selected from customary solvents that are inert under the prevailing reaction conditions. Preference is given to ethers, for example diisopropyl ether, dioxane, tetrahydrofuran, 1,2-dimethoxyethane; halogenated hydrocarbons, for example dichloromethane, chloroform, carbon tetrachloride, 1,2-dichloroethane or chlorobenzene; nitriles, for example acetonitrile or propionitrile; aromatic hydrocarbons, for example toluene or xylene; aprotic polar solvents, for example N,N-dimethylformamide or N-methylpyrrolidone, or nitrogen compounds, for example pyridine.

Suitable condensing agents are, for example, carbodiimides such as 1-(3-dimethylaminopropyl)-3-ethylcarbodiimide hydrochloride (EDCI) or 1,3-dicyclohexylcarbodiimide.

Suitable bases are inorganic bases which are typically used in such reactions. Preference is given to using bases selected by way of example from the group consisting of acetates, phosphates, carbonates and bicarbonates of alkali metals or alkaline earth metals. Particular preference is given here to sodium acetate, sodium phosphate, potassium phosphate, caesium carbonate, sodium carbonate, potassium carbonate, sodium hydrogencarbonate, potassium hydrogencarbonate.

The reaction can be carried out under reduced pressure, at standard pressure or under elevated pressure and at temperatures of 0° C. to 180° C.; with preference, the reaction is carried out at atmospheric pressure and temperatures of 20 to 140° C.

### Step b)

The compounds of the formula (IX) can be prepared by condensing the compounds of the formula (VIII), for example analogously to the processes described in WO2012/86848.

The conversion to compounds of the formula (IX) can be effected neat or in a solvent, preference being given to conducting the reaction in a solvent selected from customary solvents that are inert under the prevailing reaction conditions. Preference is given to ethers, for example diisopropyl ether, dioxane, tetrahydrofuran, 1,2-dimethoxyethane, tert-butyl methyl ether; halogenated hydrocarbons, for example dichloromethane, chloroform, carbon tetrachloride, 1,2-dichloroethane or chlorobenzene; nitriles, for example acetonitrile or propionitrile; aromatic hydrocarbons, for example toluene or xylene; aprotic polar solvents, for example N,N-dimethylformamide or N-methylpyrrolidone, or nitrogen compounds, for example pyridine.

The reaction can be carried out in the presence of a condensing agent, an acid, a base or a chlorinating agent.

Examples of suitable condensing agents are carbodiimides such as 1-(3-dimethylaminopropyl)-3-ethylcarbodiimide hydrochloride (EDCI) or 1,3-dicyclohexylcarbodiimide; anhydrides such as acetic anhydride, trifluoroacetic anhydride; a mixture of triphenylphosphine, a base and carbon tetrachloride, or a mixture of triphenylphosphine and an azo diester, for example diethylazodicarboxylic acid.

Examples of suitable acids which can be used in the reaction described are sulfonic acids such as para-toluenesulfonic acid; carboxylic acids such as acetic acid, or polyphosphoric acids.

Examples of suitable bases are nitrogen heterocycles such as pyridine, picoline, 2,6-lutidine, 1,8-diazabicyclo[5.4.0]-7-undecene (DBU); tertiary amines such as triethylamine and N,N-diisopropylethylamine; inorganic bases such as potassium phosphate, potassium carbonate and sodium hydride.

An example of a suitable chlorinating agent is phosphorus oxychloride.

The reaction can be carried out under reduced pressure, at atmospheric pressure or under elevated pressure, and at temperatures of 0° C. to 200° C.

### Step c)

The compounds of the formula (XI) can be prepared by reacting the compounds of the formula (IX) with the compounds of the formula (X) in the presence of a base.

Mercaptan derivatives of the formula (X), for example methyl mercaptan, ethyl mercaptan or isopropyl mercaptan, are either commercially available or can be prepared by known methods, for example analogously to the processes described in US2006/25633, US2006/111591, U.S. Pat. No. 2,820,062, Chemical Communications, 13 (2000), 1163-1164 or Journal of the American Chemical Society, 44 (1922), p. 1329.

The conversion to compounds of the formula (XI) can be effected neat or in a solvent, preference being given to conducting the reaction in a solvent selected from customary solvents that are inert under the prevailing reaction conditions. Preference is given to ethers, for example diisopropyl ether, dioxane, tetrahydrofuran, 1,2-dimethoxyethane, tert-butyl methyl ether; nitriles, for example acetonitrile or propionitrile; aromatic hydrocarbons, for example toluene or xylene; aprotic polar solvents, for example N,N-dimethylformamide, N-methylpyrrolidone or dimethyl sulfoxide.

Examples of suitable bases are inorganic bases from the group consisting of acetates, phosphates and carbonates of alkali metals or alkaline earth metals. Preference is given here to caesium carbonate, sodium carbonate and potassium carbonate. Further suitable bases are alkali metal hydrides, for example sodium hydride.

The reaction can be carried out under reduced pressure, at atmospheric pressure or under elevated pressure, and at temperatures of 0° C. to 200° C.

### Step d)

The compounds of the formula (XII) can be prepared by oxidizing the compounds of the formula (XI). The oxidation is generally carried out in a solvent selected from customary solvents which are inert under the prevailing reaction conditions. Preference is given to halogenated hydrocarbons, for example dichloromethane, chloroform, carbon tetrachloride, 1,2-dichloroethane or chlorobenzene; alcohols such as methanol or ethanol; formic acid, acetic acid, propionic acid or water.

Examples of suitable oxidizing agents are hydrogen peroxide, meta-chloroperbenzoic acid or sodium periodate.

The reaction can be conducted under reduced pressure, at standard pressure or under elevated pressure, and at temperatures of
−20° C. to 120° C.

### Step e)

The compounds of the formula (XIII) can be prepared by oxidizing the compounds of the formula (XII). The oxidation is generally carried out in a solvent. Preference is given to halogenated hydrocarbons, for example dichloromethane, chloroform, carbon tetrachloride, 1,2-dichloroethane or chlorobenzene; alcohols such as methanol or ethanol; formic acid, acetic acid, propionic acid or water.

Examples of suitable oxidizing agents are hydrogen peroxide and meta-chloroperbenzoic acid.

The reaction can be conducted under reduced pressure, at standard pressure or under elevated pressure, and at temperatures of
−20° C. to 120° C.

### Step f)

The compounds of the formula (XIII) can also be prepared in a one-step process by oxidizing the compounds of the formula (XI). The oxidation is generally carried out in a solvent. Preference is given to halogenated hydrocarbons, for example dichloromethane, chloroform, carbon tetrachloride, 1,2-dichloroethane or chlorobenzene; alcohols such as methanol or ethanol; formic acid, acetic acid, propionic acid or water.

Examples of suitable oxidizing agents are hydrogen peroxide and meta-chloroperbenzoic acid.

The reaction can be conducted under reduced pressure, at standard pressure or under elevated pressure, and at temperatures of
−20° C. to 120° C.

### Step g)

Compounds of the formula (I) can be prepared, for example, by reacting compounds of the formula (XIII), in which X² is preferably halogen from the group of chlorine and bromine with compounds of the formula (XIV), by methods known in the literature (see, for example, Journal of Organic Chemistry (2010), 69, 5578), for example in the presence of copper(I) iodide and basic reaction auxiliaries, for example trans-N,N′-dimethylcyclohexane-1,2-diamine and potassium carbonate, in a suitable solvent or diluent.

The required compounds of the formula (XIV) are either commercially available or can be prepared by known methods, for example analogously to the processes described in Bioorganic & Medicinal Chemistry Letters, 28 (2019), 1797-1803, Tetrahedron Letters, 47 (2006), 6743-6746, Chemical and Pharmaceutical Research, 5 (2013), 91-98, Heterocycles, 40 (1995), 851-66, WO2007/018941 or WO2015/152367.

Useful solvents or diluents include all inert organic solvents, for example aliphatic or aromatic hydrocarbons. Preference is given to using toluene.

Furthermore, the coupling can be effected from compounds of the formula (XIII) for which X² is preferably halogen from the group of fluorine, chlorine and bromine, without metal catalysis in the presence of a suitable base, for example potassium carbonate or caesium carbonate, in a suitable solvent or diluent. Suitable solvents or diluents are all inert organic solvents. Preference is given to aprotic polar solvents, for example N,N-dimethylformamide, N-methylpyrrolidone or dimethyl sulfoxide, or nitriles, for example acetonitrile or propionitrile.

The reaction according to step g) can also take place starting from compounds of the formulae (XI) or (XII).

### Methods and Uses

The invention also relates to methods of controlling animal pests, in which compounds of the formula (I) are allowed to act on animal pests and/or their habitat. The animal pests are preferably controlled in agriculture and forestry, and in material protection. This preferably excludes methods for surgical or therapeutic treatment of the human or animal body and diagnostic methods carried out on the human or animal body.

The invention further relates to the use of the compounds of formula (I) as pesticides, especially crop protection agents.

In the context of the present application, the term “pesticides” in each case also always encompasses the term “crop protection compositions”.

The compounds of the formula (I), given good plant tolerance, favourable endotherm toxicity and good environmental compatibility, are suitable for protecting plants and plant organs against biotic and abiotic stress factors, for increasing harvest yields, for improving the quality of the harvested material and for controlling animal pests, especially insects, arachnids, helminths, especially nematodes, and molluscs, which are encountered in agriculture, in horticulture, in animal husbandry, in aquatic cultures, in forests, in gardens and leisure facilities, in the protection of stored products and of materials, and in the hygiene sector.

In the context of the present patent application, the term “hygiene” should be understood to mean any and all measures, provisions and procedures which have the aim of preventing diseases, especially infection diseases, and which serve to protect the health of humans and animals and/or protect the environment and/or maintain cleanliness. According to the invention, this especially includes measures for cleaning, disinfection and sterilization, for example of textiles or hard surfaces, especially surfaces made of glass, wood, cement, porcelain, ceramic, plastic or else metal(s), in order to ensure that these are free of hygiene pests and/or their secretions. The scope of protection of the invention in this regard preferably excludes surgical or therapeutic treatment procedures to be applied to the human body or the bodies of animals, and diagnostic procedures which are conducted on the human body or the bodies of animals.

Thus, the term “hygiene sector” covers all areas, technical fields and industrial applications in which these hygiene measures, provisions and procedures are important, for example with regard to hygiene in kitchens, bakeries, airports, bathrooms, swimming pools, department stores, hotels, hospitals, stables, animal keeping, etc.

The term “hygiene pest” should therefore be understood to mean one or more animal pests whose presence in the hygiene sector is problematic, especially for reasons of health. A main aim is therefore that of avoiding, or limiting to a minimum degree, the presence of hygiene pests and/or the exposure to these in the hygiene sector. This can especially be achieved through the use of a pesticide which can be used both for prevention of infestation and to overcome an existing infestation. It is also possible to use formulations which prevent or reduce exposure to pests. Hygiene pests include, for example, the organisms mentioned below.

The term “hygiene protection” thus covers all acts by which these hygiene measures, provisions and procedures are maintained and/or improved.

The compounds of the formula (I) can preferably be used as pesticides. They are active against normally sensitive and resistant species and also against all or specific stages of development. The abovementioned pests include:
pests from the phylum of the Arthropoda, especially from the class of the Arachnida, for example, Acarus spp., e.g. Acarus siro, Aceria kuko, Aceria sheldoni, Aculops spp., Aculus spp., e.g. Aculus fockeui, Aculus schlechtendali, Amblyomma spp., Amphitetranychus viennensis, Argas spp., Boophilus spp., Brevipalpus spp., e.g. Brevipalpus phoenicis, Bryobia graminum, Bryobia praetiosa, Centruroides spp., Chorioptes spp., Dermanyssus gallinae, Dermatophagoides pteronyssinus, Dermatophagoides farinae, Dermacentor spp., Eotetranychus spp., e.g. Eotetranychus hicoriae, Epitrimerus pyri, Eutetranychus spp., e.g. Eutetranychus banksi, Eriophyes spp., e.g. Eriophyes pyri, Glycyphagus domesticus, Halotydeus destructor, Hemitarsonemus spp., e.g. Hemitarsonemus latus (=Polyphagotarsonemus latus), Hyalomma spp., Ixodes spp., Latrodectus spp., Loxosceles spp., Neutrombicula autumnalis, Nuphersa spp., Oligonychus spp., e.g. Oligonychus coffeae, Oligonychus coniferarum, Oligonychus ilicis, Oligonychus indicus, Oligonychus mangiferus, Oligonychus pratensis, Oligonychus punicae, Oligonychus yothersi, Ornithodorus spp., Ornithonyssus spp., Panonychus spp., e.g. Panonychus citri (=Metatetranychus citri), Panonychus ulmi (=Metatetranychus ulmi), Phyllocoptruta oleivora, Platytetranychus multidigituli, Polyphagotarsonemus latus, Psoroptes spp., Rhipicephalus spp., Rhizoglyphus spp., Sarcoptes spp., Scorpio maurus, Steneotarsonemus spp., Steneotarsonemus spinki, Tarsonemus spp., e.g. Tarsonemus confusus, Tarsonemus pallidus, Tetranychus spp., e.g. Tetranychus canadensis, Tetranychus cinnabarinus, Tetranychus turkestani, Tetranychus urticae, Trombicula alfreddugesi, Vaejovis spp., Vasates lycopersici;
from the class of the Chilopoda, for example, Geophilus spp., Scutigera spp.;
from the order or the class of the Collembola, for example, Onychiurus armatus, Sminthurus viridis;
from the class of the Diplopoda, for example, Blaniulus guttulatus;
from the class of the Insecta, for example from the order of the Blattodea, e.g. Blatta orientalis, Blattella asahinai, Blattella germanica, Leucophaea maderae, Loboptera decipiens, Neostylopyga rhombifolia, Panchlora spp., Parcoblatta spp., Periplaneta spp., e.g. Periplaneta americana, Periplaneta australasiae, Pycnoscelus surinamensis, Supella longipalpa;
from the order of the Coleoptera, for example, Acalymma vittatum, Acanthoscelides obtectus, Adoretus spp., Aethina tumida, Agelastica alni, Agrilus spp., e.g. Agrilus planipennis, Agrilus coxalis, Agrilus bilineatus, Agrilus anxius, Agriotes spp., e.g. Agriotes linneatus, Agriotes mancus, Agriotes obscurus, Alphitobius diaperinus, Amphimallon solstitialis, Anobium punctatum, Anomala dubia, Anoplophora spp., e.g. Anoplophora glabripennis, Anthonomus spp., e.g. Anthonomus grandis, Anthrenus spp., Apion spp., Apogonia spp., Athous haemorrhoidales, Atomaria spp., e.g. Atomaria linearis, Attagenus spp., Baris caerulescens, Bruchidius obtectus, Bruchus spp., e.g. Bruchus pisorum, Bruchus rufimanus, Cassida spp., Cerotoma trifurcata, Ceutorrhynchus spp., e.g. Ceutorrhynchus assimilis, Ceutorrhynchus quadridens, Ceutorrhynchus rapae, Chaetocnema spp., e.g. Chaetocnema confinis, Chaetocnema denticulata, Chaetocnema ectypa, Cleonus mendicus, Conoderus spp., Cosmopolites spp., e.g. Cosmopolites sordidus, Costelytra zealandica, Ctenicera spp., Curculio spp., e.g. Curculio caryae, Curculio caryatrypes, Curculio obtusus, Curculio sayi, Cryptolestes ferrugineus, Cryptolestes pusillus, Cryptorhynchus lapathi, Cryptorhynchus mangiferae, Cylindrocopturus spp., Cylindrocopturus adspersus, Cylindrocopturus furnissi, Dendroctonus spp., e.g. Dendroctonus ponderosae, Dermestes spp., Diabrotica spp., e.g. Diabrotica balteata, Diabrotica barberi, Diabrotica undecimpunctata howardi, Diabrotica undecimpunctata undecimpunctata, Diabrotica virgifera virgifera, Diabrotica virgifera zeae, Dichocrocis spp., Dicladispa armigera, Diloboderus spp., Epicaerus spp., Epilachna spp., e.g. Epilachna borealis, Epilachna varivestis, Epitrix spp., e.g. Epitrix cucumeris, Epitrix fuscula, Epitrix hirtipennis, Epitrix suberinita, Epitrix tuberis, Faustinus spp., Gibbium psylloides, Gnathocerus cornutus, Hellula undalis, Heteronychus arator, Heteronyx spp., Hoplia argentea, Hylamorpha elegans, Hylotrupes bajulus, Hypera postica, Hypomeces squamosus, Hypothenemus spp., e.g. Hypothenemus hampei, Hypothenemus obscurus, Hypothenemus pubescens, Lachnosterna consanguinea, Lasioderma serricorne, Latheticus oryzae, Lathridius spp., Lema spp., Leptinotarsa decemlineata, Leucoptera spp., e.g. Leucoptera coffeella, Limonius ectypus, Lissorhoptrus oryzophilus, Listronotus (=Hyperodes) spp., Lixus spp., Luperodes spp., Luperomorpha xanthodera, Lyctus spp., Megacyllene spp., e.g. Megacyllene robiniae, Megascelis spp., Melanotus spp., e.g. Melanotus longulus oregonensis, Meligethes aeneus, Melolontha spp., e.g. Melolontha melolontha, Migdolus spp., Monochamus spp., Naupactus xanthographus, Necrobia spp., Neogalerucella spp., Niptus hololeucus, Oryctes rhinoceros, Oryzaephilus surinamensis, Oryzaphagus oryzae, Otiorhynchus spp., e.g. Otiorhynchus cribricollis, Otiorhynchus ligustici, Otiorhynchus ovatus, Otiorhynchus rugosostriarus, Otiorhynchus sulcatus, Oulema spp., e.g. Oulema melanopus, Oulema oryzae, Oxycetonia jucunda, Phaedon cochleariae, Phyllophaga spp., Phyllophaga helleri, Phyllotreta spp., e.g. Phyllotreta armoraciae, Phyllotreta pusilla, Phyllotreta ramosa, Phyllotreta striolata, Popillia japonica, Premnotrypes spp., Prostephanus truncatus, Psylliodes spp., e.g. Psylliodes affinis, Psylliodes chrysocephala, Psylliodes punctulata, Ptinus spp., Rhizobius ventralis, Rhizopertha dominica, Rhynchophorus spp., Rhynchophorus ferrugineus, Rhynchophorus palmarum, Scolytus spp., e.g. Scolytus multistriatus, Sinoxylon perforans, Sitophilus spp., e.g. Sitophilus granarius, Sitophilus linearis, Sitophilus oryzae, Sitophilus zeamais, Sphenophorus spp., Stegobium paniceum, Sternechus spp., e.g. Sternechus paludatus, Symphyletes spp., Tanymecus spp., e.g. Tanymecus dilaticollis, Tanymecus indicus, Tanymecus palliatus, Tenebrio molitor, Tenebrioides mauretanicus, Tribolium spp., e.g. Tribolium audax, Tribolium castaneum, Tribolium confusum, Trogoderma spp., Tychius spp., Xylotrechus spp., Zabrus spp., e.g. Zabrus tenebrioides;
from the order of the Dermaptera, for example, Anisolabis maritime, Forficula auricularia, Labidura riparia; from the order of the Diptera, for example, Aedes spp., e.g. Aedes aegypti, Aedes albopictus, Aedes sticticus, Aedes vexans, Agromyza spp., e.g. Agromyza frontella, Agromyza parvicornis, Anastrepha spp., Anopheles spp., e.g. Anopheles quadrimaculatus, Anopheles gambiae, Asphondylia spp., Bactrocera spp., e.g. Bactrocera cucurbitae, Bactrocera dorsalis, Bactrocera oleae, Bibio hortulanus, Calliphora erythrocephala, Calliphora vicina, Ceratitis capitata, Chironomus spp., Chrysomya spp., Chrysops spp., Chrysozona pluvialis, Cochliomya spp., Contarinia spp., e.g. Contarinia johnsoni, Contarinia nasturtii, Contarinia pyrivora, Contarinia schulzi, Contarinia sorghicola, Contarinia tritici, Cordylobia anthropophaga, Cricotopus sylvestris, Culex spp., e.g. Culex pipiens, Culex quinquefasciatus, Culicoides spp., Culiseta spp., Cuterebra spp., Dacus oleae, Dasineura spp., e.g. Dasineura brassicae, Delia spp., e.g. Delia antiqua, Delia coaretata, Delia florilega, Delia platura, Delia radicum, Dermatobia hominis, Drosophila spp., e.g. Drosphila melanogaster, Drosophila suzukii, Echinoenemus spp., Euleia heraclei, Fannia spp., Gasterophilus spp., Glossina spp., Haematopota spp., Hydrellia spp., Hydrellia griseola, Hylemya spp., Hippobosca spp., Hypoderma spp., Liriomyza spp., e.g. Liriomyza brassicae, Liriomyza huidobrensis, Liriomyza sativae, Lucilia spp., e.g. Lucilia cuprina, Lutzomyia spp., Mansonia spp., Musca spp., e.g. Musca domestica, Musca domestica vicina, Oestrus spp., Oscinella frit, Paratanytarsus spp., Paralauterborniella subcincta, Pegomya or Pegomyia spp., e.g. Pegomya betae, Pegomya hyoscyami, Pegomya rubivora, Phlebotomus spp., Phorbia spp., Phormia spp., Piophila casei, Platyparea poeciloptera, Prodiplosis spp., Psila rosae, Rhagoletis spp., e.g. Rhagoletis cingulata, Rhagoletis completa, Rhagoletis fausta, Rhagoletis indifferens, Rhagoletis mendax, Rhagoletis pomonella, Sarcophaga spp., Simulium spp., e.g. Simulium meridionale, Stomoxys spp., Tabanus spp., Tetanops spp., Tipula spp., e.g. Tipula paludosa, Tipula simplex, Toxotrypana curvicauda;
from the order of the Hemiptera, for example, Acizzia acaciaebaileyanae, Acizzia dodonaeae, Acizzia uncatoides, Acrida turrita, Acyrthosipon spp., e.g. Acyrthosiphon pisum, Acrogonia spp., Aeneolamia spp., Agonoscena spp., Aleurocanthus spp., Aleyrodes proletella, Aleurolobus barodensis, Aleurothrixus floccosus, Allocaridara malayensis, Amrasca spp., e.g. Amrasca bigutulla, Amrasca devastans, Anuraphis cardui, Aonidiella spp., e.g. Aonidiella aurantii, Aonidiella citrina, Aonidiella inornata, Aphanostigma piri, Aphis spp., e.g. Aphis citricola, Aphis craccivora, Aphis fabae, Aphis forbesi, Aphis glycines, Aphis gossypii, Aphis hederae, Aphis illinoisensis, Aphis middletoni, Aphis nasturtii, Aphis nerii, Aphis pomi, Aphis spiraecola, Aphis viburniphila, Arboridia apicalis, Arytainilla spp., Aspidiella spp., Aspidiotus spp., e.g. Aspidiotus nerii, Atanus spp., Aulacorthum solani, Bemisia tabaci, Blastopsylla occidentalis, Boreioglycaspis melaleucae, Brachycaudus helichrysi, Brachycolus spp., Brevicoryne brassicae, Cacopsylla spp., e.g. Cacopsylla pyricola, Calligypona marginata, Capulinia spp., Carneocephala fulgida, Ceratovacuna lanigera, Cercopidae, Ceroplastes spp., Chaetosiphon fragaefolii, Chionaspis tegalensis, Chlorita onukii, Chondracris rosea, Chromaphis juglandicola, Chrysomphalus aonidum, Chrysomphalus ficus, Cicadulina mbila, Coccomytilus halli, Coccus spp., e.g. Coccus hesperidum, Coccus longulus, Coccus pseudomagnoliarum, Coccus viridis, Cryptomyzus ribis, Cryptoneossa spp., Ctenarytaina spp., Dalbulus spp., Dialeurodes chittendeni, Dialeurodes citri, Diaphorina citri, Diaspis spp., Diuraphis spp., Doralis spp., Drosicha spp., Dysaphis spp., e.g. Dysaphis apiifolia, Dysaphis plantaginea, Dysaphis tulipae, Dysmicoccus spp., Empoasca spp., e.g. Empoasca abrupta, Empoasca fabae, Empoasca maligna, Empoasca solana, Empoasca stevensi, Eriosoma spp., e.g. Eriosoma americanum, Eriosoma lanigerum, Eriosoma pyricola, Erythroneura spp., Eucalyptolyma spp., Euphyllura spp., Euscelis bilobatus, Ferrisia spp., Fiorinia spp., Furcaspis oceanica, Geococcus coffeae, Glycaspis spp., Heteropsylla cubana, Heteropsylla spinulosa, Homalodisca coagulata, Hyalopterus arundinis, Hyalopterus pruni, Icerya spp., e.g. Icerya purchasi, Idiocerus spp., Idioscopus spp., Laodelphax striatellus, Lecanium spp., e.g. Lecanium corni (=Parthenolecanium corni), Lepidosaphes spp., e.g. Lepidosaphes ulmi, Lipaphis erysimi, Lopholeucaspis japonica, Lycorma delicatula, Macrosiphum spp., e.g. Macrosiphum euphorbiae, Macrosiphum lilii, Macrosiphum rosae, Macrosteles facifrons, Mahanarva spp., Melanaphis sacchari, Metcalfiella spp., Metcalfa pruinosa, Metopolophium dirhodum, Monellia costalis, Monelliopsis pecanis, Myzus spp., e.g. Myzus ascalonicus, Myzus cerasi, Myzus ligustri, Myzus ornatus, Myzus persicae, Myzus nicotianae, Nasonovia ribisnigri, Neomaskellia spp., Nephotettix spp., e.g. Nephotettix cincticeps, Nephotettix nigropictus, Nettigoniclla spectra, Nilaparvata lugens, Oncometopia spp., Orthezia praelonga, Oxya chinensis, Pachypsylla spp., Parabemisia myricae, Paratrioza spp., e.g. Paratrioza cockerelli, Parlatoria spp., Pemphigus spp., e.g. Pemphigus bursarius, Pemphigus populivenae, Peregrinus maidis, Perkinsiella spp., Phenacoccus spp., e.g. Phenacoccus madeirensis, Phloeomyzus passerinii, Phorodon humuli, Phylloxera spp., e.g. Phylloxera devastatrix, Phylloxera notabilis, Pinnaspis aspidistrae, Planococcus spp., e.g. Planococcus citri, Prosopidopsylla flava, Protopulvinaria pyriformis, Pseudaulacaspis pentagona, Pseudococcus spp., e.g. Pseudococcus calceolariae, Pseudococcus comstocki, Pseudococcus longispinus, Pseudococcus maritimus, Pseudococcus viburni, Psyllopsis spp., Psylla spp., e.g. Psylla buxi, Psylla mali, Psylla pyri, Pteromalus spp., Pulvinaria spp., Pyrilla spp., Quadraspidiotus spp., e.g. Quadraspidiotus juglansregiae, Quadraspidiotus ostreaeformis, Quadraspidiotus perniciosus, Quesada gigas, Rastrococcus spp., Rhopalosiphum spp., e.g. Rhopalosiphum maidis, Rhopalosiphum oxyacanthae, Rhopalosiphum padi, Rhopalosiphum rufiabdominale, Saissetia spp., e.g. Saissetia coffeae, Saissetia miranda, Saissetia neglecta, Saissetia oleae, Scaphoideus titanus, Schizaphis graminum, Selenaspidus articulatus, Sipha flava, Sitobion avenae, Sogata spp., Sogatella furcifera, Sogatodes spp., Stictocephala festina, Siphoninus phillyreae, Tenalaphara malayensis, Tetragonocephela spp., Tinocallis caryaefoliae, Tomaspis spp., Toxoptera spp., e.g. Toxoptera aurantii, Toxoptera citricidus, Trialeurodes vaporariorum, Trioza spp., e.g. Trioza diospyri, Typhlocyba spp., Unaspis spp., Viteus vitifolii, Zygina spp.;
from the suborder of the Heteroptera, for example, Aelia spp., Anasa tristis, Antestiopsis spp., Boisea spp., Blissus spp., Calocoris spp., Campylomma livida, Cavelerius spp., Cimex spp., e.g. Cimex adjunctus, Cimex hemipterus, Cimex lectularius, Cimex pilosellus, Collaria spp., Creontiades dilutus, Dasynus piperis, Dichelops furcatus, Diconocoris hewetti, Dysdercus spp., Euschistus spp., e.g. Euschistus heros, Euschistus servus, Euschistus tristigmus, Euschistus variolarius, Eurydema spp., Eurygaster spp., Halyomorpha halys, Heliopeltis spp., Horcias nobilellus, Leptocorisa spp., Leptocorisa varicornis, Leptoglossus occidentalis, Leptoglossus phyllopus, Lygocoris spp., e.g. Lygocoris pabulinus, Lygus spp., e.g. Lygus elisus, Lygus hesperus, Lygus lineolaris, Macropes excavatus, Megacopta cribraria, Miridae, Monalonion atratum, Nezara spp., e.g. Nezara viridula, Nysius spp., Oebalus spp., Pentomidae, Piesma quadrata, Piezodorus spp., e.g. Piezodorus guildinii, Psallus spp., Pseudacysta persea, Rhodnius spp., Sahlbergella singularis, Scaptocoris castanea, Scotinophora spp., Stephanitis nashi, Tibraca spp., Triatoma spp.;
from the order of the Hymenoptera, for example, Acromyrmex spp., Athalia spp., e.g. Athalia rosae, Atta spp., Camponotus spp., Dolichovespula spp., Diprion spp., e.g. Diprion similis, Hoplocampa spp., e.g. Hoplocampa cookei, Hoplocampa testudinea, Lasius spp., Linepithema (Iridiomyrmex) humile, Monomorium pharaonis, Paratrechina spp., Paravespula spp., Plagiolepis spp., Sirex spp., e.g. Sirex noctilio, Solenopsis invicta, Tapinoma spp., Technomyrmex albipes, Urocerus spp., Vespa spp., e.g. Vespa crabro, Wasmannia auropunctata, Xeris spp.; from the order of the Isopoda, for example, Armadillidium vulgare, Oniscus asellus, Porcellio scaber;
from the order of the Isoptera, for example, Coptotermes spp., e.g. Coptotermes formosanus, Cornitermes cumulans, Cryptotermes spp., Incisitermes spp., Kalotermes spp., Microtermes obesi, Nasutitermes spp., Odontotermes spp., Porotermes spp., Reticulitermes spp., e.g. Reticulitermes flavipes, Reticulitermes hesperus;
from the order of the Lepidoptera, for example, Achroia grisella, Acronicta major, Adoxophyes spp., e.g. Adoxophyes orana, Aedia leucomelas, Agrotis spp., e.g. Agrotis segetum, Agrotis ipsilon, Alabama spp., e.g. Alabama argillacea, Amyelois transitella, Anarsia spp., Anticarsia spp., e.g. Anticarsia gemmatalis, Argyroploce spp., Autographa spp., Barathra brassicae, Blastodacna atra, Borbo cinnara, Bucculatrix thurberiella, Bupalus piniarius, Busseola spp., Cacoecia spp., Caloptilia theivora, Capua reticulana, Carpocapsa pomonella, Carposina niponensis, Cheimatobia brumata, Chilo spp., e.g. Chilo plejadellus, Chilo suppressalis, Choreutis pariana, Choristoneura spp., Chrysodeixis chalcites, Clysia ambiguella, Cnaphalocerus spp., Cnaphalocrocis medinalis, Cnephasia spp., Conopomorpha spp., Conotrachelus spp., Copitarsia spp., Cydia spp., e.g. Cydia nigricana, Cydia pomonella, Dalaca noctuides, Diaphania spp., Diparopsis spp., Diatraea saccharalis, Dioryctria spp., e.g. Dioryctria zimmermani, Earias spp., Ecdytolopha aurantium, Elasmopalpus lignosellus, Eldana saccharina, Ephestia spp., e.g. Ephestia elutella, Ephestia kuehniella, Epinotia spp., Epiphyas postvittana, Erannis spp., Erschoviella musculana, Etiella spp., Eudocima spp., Eulia spp., Eupoecilia ambiguella, Euproctis spp., e.g. Euproctis chrysorrhoea, Euxoa spp., Feltia spp., Galleria mellonella, Gracillaria spp., Grapholitha spp., e.g. Grapholita molesta, Grapholita prunivora, Hedylepta spp., Helicoverpa spp., e.g. Helicoverpa armigera, Helicoverpa zea, Heliothis spp., e.g. Heliothis virescens, Hepialus spp., e.g. Hepialus humuli, Hofmannophila pseudospretella, Homoeosoma spp., Homona spp., Hyponomeuta padella, Kakivoria flavofasciata, Lampides spp., Laphygma spp., Laspeyresia molesta, Leucinodes orbonalis, Leucoptera spp., e.g. Leucoptera coffeella, Lithocolletis spp., e.g. Lithocolletis blancardella, Lithophane antennata, Lobesia spp., e.g. Lobesia botrana, Loxagrotis albicosta, Lymantria spp., e.g. Lymantria dispar, Lyonetia spp., e.g. Lyonetia clerkella, Malacosoma neustria, Maruca testulalis, Mamestra brassicae, Melanitis leda, Mocis spp., Monopis obviella, Mythimna separata, Nemapogon cloacellus, Nymphula spp., Oiketicus spp., Omphisa spp., Operophtera spp., Oria spp., Orthaga spp., Ostrinia spp., e.g. Ostrinia nubilalis, Panolis flammea, Parnara spp., Pectinophora spp., e.g. Pectinophora gossypiella, Perileucoptera spp., Phthorimaea spp., e.g. Phthorimaea operculella, Phyllocnistis citrella, Phyllonorycter spp., e.g. Phyllonorycter blancardella, Phyllonorycter crataegella, Pieris spp., e.g. Pieris rapae, Platynota stultana, Plodia interpunctella, Plusia spp., Plutella xylostella (=Plutella maculipennis), Podesia spp., e.g. Podesia syringae, Prays spp., Prodenia spp., Protoparce spp., Pseudaletia spp., e.g. Pseudaletia unipuncta, Pseudoplusia includens, Pyrausta nubilalis, Rachiplusia nu, Schoenobius spp., e.g. Schoenobius bipunctifer, Scirpophaga spp., e.g. Scirpophaga innotata, Scotia segetum, Sesamia spp., e.g. Sesamia inferens, Sparganothis spp., Spodoptera spp., e.g. Spodoptera eradiana, Spodoptera exigua, Spodoptera frugiperda, Spodoptera praefica, Stathmopoda spp., Stenoma spp., Stomopteryx subsecivella, Synanthedon spp., Tecia solanivora, Thaumetopoea spp., Thermesia gemmatalis, Tinea cloacella, Tinea pellionella, Tineola bisselliella, Tortrix spp., Trichophaga tapetzella, Trichoplusia spp., e.g. Trichoplusia ni, Tryporyza incertulas, Tuta absoluta, Virachola spp.;
from the order of the Orthoptera or Saltatoria, for example, Acheta domesticus, Dichroplus spp., Gryllotalpa spp., e.g. Gryllotalpa gryllotalpa, Hieroglyphus spp., Locusta spp., e.g. Locusta migratoria, Melanoplus spp., e.g. Melanoplus devastator, Paratlanticus ussuriensis, Schistocerca gregaria;
from the order of the Phthiraptera, for example, Damalinia spp., Haematopinus spp., Linognathus spp., Pediculus spp., Phylloxera vastatrix, Phthirus pubis, Trichodectes spp.;
from the order of the Psocoptera, for example, Lepinotus spp., Liposcelis spp.;
from the order of the Siphonaptera, for example, Ceratophyllus spp., Ctenocephalides spp., e.g. Ctenocephalides canis, Ctenocephalides felis, Pulex irritans, Tunga penetrans, Xenopsylla cheopis;
from the order of the Thysanoptera, for example, Anaphothrips obscurus, Baliothrips biformis, Chaetanaphothrips leeuweni, Drepanothrips reuteri, Enneothrips flavens, Frankliniella spp., e.g. Frankliniella fusca, Frankliniella occidentalis, Frankliniella schultzei, Frankliniella tritici, Frankliniella vaccinii, Frankliniella williamsi, Haplothrips spp., Heliothrips spp., Hercinothrips femoralis, Kakothrips spp., Rhipiphorothrips cruentatus, Scirtothrips spp., Taeniothrips cardamomi, Thrips spp., e.g. Thrips palmi, Thrips tabaci;
from the order of the Zygentoma (=Thysanura), for example, Ctenolepisma spp., Lepisma saccharina, Lepismodes inquilinus, Thermobia domestica;
from the class of the Symphyla, for example, Scutigerella spp., e.g. Scutigerella immaculata;
pests from the phylum of the Mollusca, for example from the class of the Bivalvia, e.g. Dreissena spp.;
and also from the class of the Gastropoda, for example, Arion spp., e.g. Arion ater rufus, Biomphalaria spp., Bulinus spp., Deroceras spp., e.g. Deroceras laeve, Galba spp., Lymnaea spp., Oncomelania spp., Pomacea spp., Succinea spp.;
plant pests from the phylum of the Nematoda, i.e. plant-parasitic nematodes, in particular Aglenchus spp., for example Aglenchus agricola, Anguina spp., for example Anguina tritici, Aphelenchoides spp., for example Aphelenchoides arachidis, Aphelenchoides fragariae, Belonolaimus spp., for example Belonolaimus gracilis, Belonolaimus longicaudatus, Belonolaimus nortoni, Bursaphelenchus spp., for example Bursaphelenchus cocophilus, Bursaphelenchus eremus, Bursaphelenchus xylophilus, Cacopaurus spp., for example Cacopaurus pestis, Criconemella spp., for example Criconemella curvata, Criconemella onoensis, Criconemella ornata, Criconemella rusium, Criconemella xenoplax (=Mesocriconema xenoplax), Criconemoides spp., for example Criconemoides ferniae, Criconemoides onoense, Criconemoides ornatum, Ditylenchus spp., for example Ditylenchus dipsaci, Dolichodorus spp., Globodera spp., for example Globodera pallida, Globodera rostochiensis, Helicotylenchus spp., for example Helicotylenchus dihystera, Hemicriconemoides spp., Hemicycliophora spp., Heterodera spp., for example Heterodera avenae, Heterodera glycines, Heterodera schachtii, Hirschmaniella spp., Hoplolaimus spp., Longidorus spp., for example Longidorus africanus, Meloidogyne spp., for example Meloidogyne chitwoodi, Meloidogyne fallax, Meloidogyne hapla, Meloidogyne incognita, Meloinema spp., Nacobbus spp., Neotylenchus spp., Paralongidorus spp., Paraphelenchus spp., Paratrichodorus spp., for example Paratrichodorus minor, Paratylenchus spp., Pratylenchus spp., for example Pratylenchus penetrans, Pseudohalenchus spp., Psilenchus spp., Punctodera spp., Quinisulcius spp., Radopholus spp., for example Radopholus citrophilus, Radopholus similis, Rotylenchulus spp., Rotylenchus spp., Scutellonema spp., Subanguina spp., Trichodorus spp., for example Trichodorus obtusus, Trichodorus primitivus, Tylenchorhynchus spp., for example Tylenchorhynchus annulatus, Tylenchulus spp., for example Tylenchulus semipenetrans, Xiphinema spp., for example Xiphinema index.

The compounds of the formula (I) can, as the case may be, in certain concentrations or application rates, also be used as herbicides, safeners, growth regulators or compositions to improve plant properties, as microbicides or gametocides, for example as fungicides, antimycotics, bactericides, viricides (including agents against viroids) or as agents against MLO (mycoplasma-like organisms) and RLO (rickettsia-like organisms). They can, as the case may be, also be used as intermediates or precursors for the synthesis of other active ingredients.

### Formulations/Use Forms

The present invention further relates to formulations, especially formulations for controlling unwanted animal pests. The formulation can be applied to the animal pest and/or its habitat.

The formulation of the invention can be provided as a ready-to-use “use form”, i.e. the formulations can be applied directly to the plants or seeds using a suitable device such as a sprayer or duster. Alternatively, the formulations can be provided to the end user in the form of concentrates to be diluted prior to use, preferably with water. Unless indicated otherwise, the term “formulation” refers to such a concentrate, whereas the term “use form” refers to a solution which is ready to use for the end user, i.e. usually such a dilute formulation.

The formulation of the invention can be prepared in a customary manner, for example by mixing the compound of the invention with one or more suitable auxiliaries, for example those disclosed herein.

The formulation comprises at least one compound of the invention and at least one agriculturally useful auxiliary, e.g. carrier and/or surfactant(s).

The carrier is a solid or liquid, natural or synthetic, organic or inorganic substance which is generally inert. The carrier generally improves the application of the compounds, for example to plants, parts of plants or seeds. Examples of suitable solid carriers include, without limitation, ammonium salts, in particular ammonium sulfates, ammonium phosphates and ammonium nitrates, ground natural minerals, such as kaolins, clays, talc, chalk, quartz, attapulgite, montmorillonite and diatomaceous earth, silica gel and ground synthetic minerals such as finely divided silica, alumina and silicates. Examples of typical suitable solid carriers for preparing granules are, without limitation, crushed and fractionated natural minerals such as calcite, marble, pumice, sepiolite and dolomite, synthetic granules of inorganic and organic meals and granules of organic materials such as paper, sawdust, coconut shells, maize cobs and tobacco stalks. Examples of suitable liquid carriers include, without limitation, water, organic solvents and combinations thereof. Examples of suitable solvents include polar and nonpolar organic chemical liquids, for example from the classes of the aromatic and nonaromatic hydrocarbons (such as cyclohexane, paraffins, alkylbenzenes, xylene, toluene, tetrahydronaphthalene, alkylnaphthalenes, chlorinated aromatics or chlorinated aliphatic hydrocarbons such as chlorobenzenes, chloroethylenes or methylene chloride), alcohols and polyols (which may also be substituted, etherified and/or esterified, such as ethanol, propanol, butanol, benzyl alcohol, cyclohexanol or glycol), ketones (such as acetone, methyl ethyl ketone, methyl isobutyl ketone, acetophenone or cyclohexanone), esters (including fats and oils) and (poly)ethers, unsubstituted and substituted amines, amides (such as dimethylformamide or fatty amides) and esters thereof, lactams (such as N-alkylpyrrolidones, in particular N-methylpyrrolidone) and lactones, sulfones and sulfoxides (such as dimethyl sulfoxide), oils of vegetable or animal origin, nitriles (alkyl nitriles such as acetonitrile, propionitrile, butyronitrile or aromatic nitriles such as benzonitrile), carbonates (cyclic carbonates such as ethylene carbonate, propylene carbonate, butylene carbonate, or dialkyl carbonates such as dimethyl carbonate, diethyl carbonate, dipropyl carbonate, dibutyl carbonate, dioctyl carbonate). The carrier may also be a liquefied gaseous extender, i.e. a liquid which is gaseous at ambient temperature and under atmospheric pressure, for example an aerosol propellant such as halogenated hydrocarbons, butane, propane, nitrogen and carbon dioxide.

Preferred solid carriers are selected from clays, talc and silica.

Preferred liquid carriers are selected from water, fatty amides and esters thereof, aromatic and nonaromatic hydrocarbons, lactams, lactones, carbonates, ketones and (poly)ethers.

The amount of carrier is typically in the range from 1 to 99.99% by weight, preferably 5 to 99.9% by weight, particularly preferably 10 to 99.5% by weight and most preferably 20 to 99% by weight of the formulation.

Liquid carriers are typically present in a range of from 20 to 90% by weight, for example 30 to 80% by weight, of the formulation.

Solid carriers are typically present in a range of from 0 to 50% by weight, preferably 5 to 45% by weight, for example 10 to 30% by weight, of the formulation.

If the formulation comprises two or more carriers, the ranges defined refer to the total amount of carrier.

The surfactant can be an ionic (cationic or anionic), amphoteric or non-ionic surfactant such as ionic or non-ionic emulsifiers, foam-formers, dispersants, wetting agents, penetrants and any mixtures thereof. Examples of suitable surfactants include, without limitation, salts of polyacrylic acid, ethoxylated poly(alpha-substituted)acrylate derivatives, salts of lignosulfonic acid (such as sodium lignosulfonate), salts of phenolsulfonic acid or naphthalenesulfonic acid, polycondensates of ethylene oxide and/or propylene oxide with or without alcohols, fatty acids or fatty amines (for example polyoxyethylene fatty acid esters such as castor oil ethoxylate, polyoxyethylene fatty alcohol ether, for example alkylaryl polyglycol ether), substituted phenols (preferably alkylphenols or arylphenols), salts of sulfosuccinic esters, taurine derivatives (preferably alkyl taurates), phosphoric esters of polyethoxylated alcohols or phenols, fatty acid esters of polyols (such as fatty acid esters of glycerol, sorbitol or sucrose), sulfates (such as alkyl sulfates and alkylether sulfates), sulfonates (for example alkylsulfonates, arylsulfonates and alkylbenzenesulfonates), sulfonated polymers of naphthalene/formaldehyde, phosphate esters, protein hydrolysates, lignosulfite waste liquors and methylcellulose. If in the present paragraph reference is made to salts, this preferably refers to the relevant alkali metal, alkaline earth metal and ammonium salts.

Preferred surfactants are selected from ethoxylated poly(alpha-substituted)acrylate derivatives, polycondensates of ethylene oxide and/or propylene oxide with alcohols, polyoxyethylene fatty acid esters, alkylbenzenesulfonates, sulfonated polymers of naphthalene/formaldehyde, polyoxyethylene fatty acid esters such as castor oil ethoxylate, sodium lignosulfonate and arylphenol ethoxylate.

The amount of surfactant is typically in the range of from 5 to 40% by weight, for example 10 to 20% by weight, of the formulation.

Further examples of suitable auxiliaries include water-repellent substances, drying agents, binders (adhesives, tackifiers, fixatives such as carboxymethylcellulose, natural and synthetic polymers in the form of powders, granules or latices, such as gum arabic, polyvinyl alcohol and polyvinyl acetate, natural phospholipids such as cephalins and lecithins and synthetic phospholipids, polyvinylpyrrolidone and tylose), thickeners and secondary thickeners (such as cellulose ethers, acrylic acid derivatives, xanthan gum, modified clays, e.g. the products available under the name Bentone, and finely divided silica), stabilizers (e.g. cold stabilizers, preservatives (e.g. dichlorophen, benzyl alcohol hemiformal, 1,2-benzisothiazolin-3-one, 2-methyl-4-isothiazolin-3-one), antioxidants, sunscreens, in particular UV absorbers, and other agents which improve chemical and/or physical stability), dyes or pigments (such as inorganic pigments, e.g. iron oxide, titanium oxide and Prussian blue; organic dyes, e.g. alizarin, azo and metalphthalocyanine dyes), antifoams (e.g. silicone antifoams and magnesium stearate), antifreeze agents, adhesives, gibberellins and processing aids, mineral and vegetable oils, fragrances, waxes, nutrients (including trace nutrients such as salts of iron, manganese, boron, copper, cobalt, molybdenum and zinc), protective colloids, thixotropic substances, penetrants, sequestrants and complex formers.

The choice of auxiliaries depends on the intended application of the compound of the invention and/or on the physical properties of the compound(s). Furthermore, auxiliaries may be chosen such that they impart certain properties (technical, physical and/or biological properties) to the formulations or the use forms prepared therefrom. By appropriate selection of auxiliaries, it is possible to adapt the formulations to certain requirements.

The formulation comprises an insecticidally/acaricidally/nematicidally effective amount of the compound(s) of the invention. The term “effective amount” refers to an amount which is sufficient for controlling harmful insects/mites/nematodes on cultivated plants or in the protection of materials and causes no substantial damage to the treated plants. Such an amount may vary within a wide range and depends on various factors such as the species of insect/mite/nematode to be controlled, on the treated cultivated plant or the treated material, on the climatic conditions and on the compound of the invention used in each case. Usually, the formulation of the invention comprises 0.01 to 99% by weight, preferably 0.05 to 98% by weight, more preferably 0.1 to 95% by weight, even more preferably 0.5 to 90% by weight, most preferably 1 to 80% by weight, of the compound of the invention. It is possible for a formulation to comprise two or more compounds of the invention. In such a case, the ranges defined refer to the total amount of the compounds according to the present invention.

The formulation of the invention may take the form of any conventional formulation type, such as solutions (e.g. aqueous solutions), emulsions, water- and oil-based suspensions, powders (e.g. wettable powders, soluble powders), dusts, pastes, granules (e.g. soluble granules, granules for broadcasting), suspoemulsion concentrates, natural or synthetic products impregnated with the compound of the invention, fertilizers and also microencapsulations in polymeric substances. The compound of the invention may be in suspended, emulsified or dissolved form. Examples of particular suitable formulation types are solutions, water-soluble concentrates (e.g. SL, LS), dispersion concentrates (DC), suspensions and suspension concentrates (e.g. SC, OD, OF, FS), emulsion concentrates (e.g. EC), emulsions (e.g. EW, EO, ES, ME, SE), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN) and gel formulations for treating plant propagation material such as seeds (e.g. GW, GF). These and other formulation types have been defined by the Food and Agriculture Organization of the United Nations (FAO). A review can be found in the “Catalogue of pesticide formulation types and international coding system”, Technical Monograph no. 2, 6. ed., May 2008, Croplife International.

Preferably, the formulation of the invention takes the form of one of the following types: EC, SC, FS, SE, OD, WG, WP, CS, particularly preferably EC, SC, OD, WG, CS.

Further details with respect to examples of formulation types and their preparation are given below. If two or more compounds of the invention are present, the defined amount of compound of the invention refers to the total amount of the compounds of the present invention. This also applies conversely to all further components of the formulation if two or more representatives of such a component, for example a wetting agent or binder, are present.

### i) Water-Soluble Concentrates (SL, LS)

10-60% by weight of at least one compound of the invention and 5-15% by weight of surfactant (e.g. polycondensates of ethylene oxide and/or propylene oxide with alcohols) are dissolved in such an amount of water and/or water-soluble solvent (e.g. alcohols such as propylene glycol and carbonates such as propylene carbonate) as to result in a total amount of 100%. Before application, the concentrate is diluted with water.

### ii) Dispersion Concentrates (DC)

5-25% by weight of at least one compound of the invention and 1-10% by weight of surfactant and/or binder (e.g. polyvinylpyrrolidone) are dissolved in such an amount of organic solvent (e.g. cyclohexane) as to result in a total amount of 100% by weight. Dilution with water gives a dispersion.

iii) Emulsion Concentrates (EC)

15-70% by weight of at least one compound of the invention and 5-10% by weight of surfactant (e.g. a mixture of calcium dodecylbenzenesulfonate and castor oil ethoxylate) are dissolved in such an amount of water-insoluble organic solvent (e.g. aromatic hydrocarbon or fatty acid amide) and, if required, additional water-soluble solvent as to result in a total amount of 100% by weight. Dilution with water gives an emulsion.

### iv) Emulsions (EW, EO, ES)

5-40% by weight of at least one compound of the invention and 1-10% by weight of surfactant (e.g. a mixture of calcium dodecylbenzenesulfonate and castor oil ethoxylate, or polycondensates of ethylene oxide and/or propylene oxide with or without alcohols) are dissolved in 20-40% by weight of water-insoluble organic solvent (e.g. aromatic hydrocarbon). Using an emulsifying machine, the mixture is added to such an amount of water as to result in a total amount of 100% by weight. The formulation obtained is a homogeneous emulsion. Prior to application, the emulsion may be diluted further with water.

### v) Suspensions and Suspension Concentrates

v-1) Water-based (SC, FS)

In a suitable mill, for example a ball mill, 20-60% by weight of at least one compound of the invention, with addition of 2-10% by weight of surfactant (e.g. sodium lignosulfonate and polyoxyethylene fatty alcohol ether), 0.1-2% by weight of thickener (e.g. xanthan gum) and water, is comminuted to give a fine active ingredient suspension. The water is added in such an amount as to result in a total amount of 100% by weight. Dilution with water gives a stable suspension of the active ingredient. For formulations of the FS type, up to 40% by weight of binder (e.g. polyvinyl alcohol) are added.

v-2) Oil-Based (OD, OF)

In a suitable mill, for example a ball mill, 20-60% by weight of at least one compound of the invention, with addition of 2-10% by weight of surfactant (e.g. sodium lignosulfonate and polyoxyethylene fatty alcohol ether), 0.1-2% by weight of thickener (e.g. modified clay, especially Bentone, or silica) and an organic carrier, is comminuted to give a fine active ingredient/oil suspension. The organic carrier is added in such an amount as to result in a total amount of 100% by weight. Dilution with water gives a stable dispersion of the active ingredient.

### vi) Water-Dispersible Granules and Water-Soluble Granules (WG, SG)

1-90% by weight, preferably 20-80% by weight, most preferably 50-80% by weight of at least one compound of the invention, with addition of a surfactant (e.g. sodium lignosulfonate and sodium alkylnaphthylsulfonates) and optionally carrier material, is finely ground and converted to water-dispersible or water-soluble granules by typical industrial processes such as extrusion, spray drying, fluidized-bed granulation.

Surfactant and carrier material are used in such an amount as to result in a total amount of 100% by weight. Dilution with water gives a stable dispersion or solution of the active ingredient.

vii) Water-Dispersible Powders and Water-Soluble Powders (WP, SP, WS)

50-80% by weight of at least one compound of the invention is ground in a rotor/stator mill with addition of 1-20% by weight of surfactant (e.g. sodium lignosulfonate, sodium alkylnaphthylsulfonates) and such an amount of solid carrier, e.g. silica gel, as to result in a total amount of 100% by weight. Dilution with water gives a stable dispersion or solution of the active ingredient.

viii) Gel (GW, GF)

In a ball mill, 5-25% by weight of at least one compound of the invention is comminuted with addition of 3-10% by weight of surfactant (e.g. sodium lignosulfonate), 1-5% by weight of binder (e.g. carboxymethylcellulose) and such an amount of water as to result in a total amount of 100% by weight. This affords a fine suspension of the active ingredient. Dilution with water gives a stable suspension of the active ingredient.

### ix) Microemulsion (ME)

5-20% by weight of at least one compound of the invention is added to 5-30% by weight of organic solvent mixture (e.g. fatty acid dimethylamide and cyclohexanone), 10-25% by weight of surfactant mixture (e.g. polyoxyethylene fatty alcohol ether and arylphenol ethoxylate) and such an amount of water as to result in a total amount of 100% by weight. This mixture is stirred for 1 h, resulting in the spontaneous formation of a thermodynamically stable microemulsion.

### x) Microcapsules (CS)

An oil phase comprising 5-50% by weight of at least one compound of the invention, 0-40% by weight of water-insoluble organic solvent (e.g. aromatic hydrocarbon), 2-15% by weight of acrylic monomers (e.g. methyl methacrylate, methacrylic acid and a di- or triacrylate) is dispersed in an aqueous solution of a protective colloid (e.g. polyvinyl alcohol). A free-radical polymerization initiated with a free-radical initiator leads to the formation of poly(meth)acrylate microcapsules. Alternatively, an oil phase comprising 5-50% by weight of at least one compound of the invention, 0-40% by weight of water-insoluble organic solvent (e.g. aromatic hydrocarbon) and an isocyanate monomer (e.g. diphenylmethane 4,4′-diisocyanate) is dispersed in an aqueous solution of a protective colloid (e.g. polyvinyl alcohol), which leads to the formation of polyurea microcapsules. If appropriate, it is also possible to add a polyamine (e.g. hexamethylenediamine) to induce the formation of polyurea microcapsules. The monomers account for 1-10% by weight of the total CS formulation.

### xi) Dusting Powders (DP, DS)

1-10% by weight of at least one compound of the invention is finely ground and mixed intimately with such an amount of solid carrier, e.g. finely divided kaolin, as to result in a total amount of 100% by weight.

xii) Granules (GR, FG)

0.5-30% by weight of at least one compound of the invention is finely ground and associated with such an amount of solid carrier (e.g. silicate) as to result in a total amount of 100% by weight.

xiii) Ultra-Low Volume Liquids (UL)

1-50% by weight of at least one compound of the invention is dissolved in such an amount of organic solvent, e.g. aromatic hydrocarbon, as to result in a total amount of 100% by weight.

The formulation types i) to xiii) may comprise further auxiliaries such as 0,1-1% by weight of preservatives, 0.1-1% by weight of antifoams, 0.1-1% by weight of dyes and/or pigments and 5-10% by weight of antifreezes.

### Mixtures

The compounds of the formula (I) can also be used in a mixture with one or more suitable fungicides, bactericides, acaricides, molluscicides, nematicides, insecticides, microbiological agents, beneficial organisms, herbicides, fertilizers, bird repellents, phytotonics, sterilants, safeners, semiochemicals and/or plant growth regulators, in order thus, for example, to broaden the spectrum of action, prolong the period of action, enhance the rate of action, prevent repellency or prevent evolution of resistance. In addition, active ingredient combinations of this kind can improve plant growth and/or tolerance to abiotic factors, for example high or low temperatures, to drought or to elevated water content or soil salinity. It is also possible to improve flowering and fruiting performance, optimize germination capacity and root development, facilitate harvesting and improve yields, influence maturation, improve the quality and/or the nutritional value of the harvested products, prolong storage life and/or improve the processibility of the harvested products.

In addition, the compounds of the formula (I) may be present in a mixture with other active ingredients or semiochemicals such as attractants and/or bird repellents and/or plant activators and/or growth regulators and/or fertilizers. Likewise, the compounds of the formula (I) can be used to improve plant properties, for example growth, yield and quality of the harvested material.

In a particular embodiment of the invention, the compounds of the formula (I) are present in formulations or in the use forms prepared from these formulations in a mixture with further compounds, preferably those as described below.

If one of the compounds mentioned below can occur in different tautomeric forms, these forms are also included even if not explicitly mentioned in each case. All the mixing components mentioned, as the case may be, may also form salts with suitable bases or acids if they are capable of doing so on the basis of their functional groups.

### Insecticides/Acaricides/Nematicides

The active ingredients specified here with their common names are known and are described for example in “The Pesticide Manual”, 16th ed., British Crop Protection Council 2012, or can be searched for on the Internet (e.g. http://www.alanwood.net/pesticides). The classification is based on the IRAC Mode of Action Classification Scheme applicable at the time of filing of this patent application.

(1) Acetylcholinesterase (AChE) inhibitors, preferably carbamates selected from alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC and xylylcarb; or organophosphates selected from acephate, azamethiphos, azinphos-ethyl, azinphos-methyl, cadusafos, chlorethoxyfos, chlorfenvinphos, chlormephos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothiophosphoryl) salicylate, isoxathion, malathion, mecarbam, methamidophos, methidathion, mevinphos, monocrotophos, naled, omethoate, oxydemeton-methyl, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, triclorfon and vamidothion.

(2) GABA-gated chloride channel blockers, preferably cyclodiene-organochlorines selected from chlordane and endosulfan, or phenylpyrazoles (fiproles) selected from ethiprole and fipronil.

(3) Sodium channel modulators, preferably pyrethroids selected from acrinathrin, allethrin, d-cis-trans allethrin, d-trans allethrin, bifenthrin, bioallethrin, bioallethrin S-cyclopentenyl isomer, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(1R)-trans isomer], deltamethrin, empenthrin [(EZ)-(1R) isomer], esfenvalerate, etofenprox, fenpropathrin, fenvalerate, flucythrinate, flumethrin, tau-fluvalinate, halfenprox, imiprothrin, kadethrin, momfluorothrin, permethrin, phenothrin [(1R)-trans isomer], prallethrin, pyrethrins (pyrethrum), resmethrin, silafluofen, tefluthrin, tetramethrin, tetramethrin [(1R) isomer], tralomethrin and transfluthrin or DDT or methoxychlor.

(4) Competitive modulators of the nicotinic acetylcholine receptor (nAChR), preferably neonicotinoids selected from acetamiprid, clothianidin, dinotefuran, imidacloprid, nitenpyram, thiacloprid and thiamethoxam, or nicotine, or sulfoximines selected from sulfoxaflor, or butenolides selected from flupyradifurone, or mesoionics selected from triflumezopyrim.

(5) Nicotinic acetylcholine receptor (nAChR) allosteric modulators, preferably spinosyns selected from spinetoram and spinosad.

(6) Glutamate-gated chloride channel (GluCl) allosteric modulators, preferably avermectins/milbemycins selected from abamectin, emamectin benzoate, lepimectin and milbemectin.

(7) Juvenile hormone mimics, preferably juvenile hormone analogues selected from hydroprene, kinoprene and methoprene, or fenoxycarb or pyriproxyfen.

(8) Miscellaneous non-specific (multi-site) inhibitors, preferably alkyl halides selected from methyl bromide and other alkyl halides; or chloropicrin or sulfuryl fluoride or borax or tartar emetic or methyl isocyanate generators selected from diazomet and metam.

(9) TRPV channel modulators of chordotonal organs, preferably pyridinazomethanes selected from pymetrozine and pyrifluquinazon, or pyropenes selected from afidopyropen.

(10) CHS1-related mite growth inhibitors selected from clofentezine, hexythiazox, diflovidazin and etoxazole.

(11) Microbial disruptors of insect midgut membranes selected from Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis, and B.t. plant proteins selected from Cry1Ab, Cry1Ac, CrylFa, Cry1A.105, Cry2Ab, VIP3A, mCry3A, Cry3Ab, Cry3Bb and Cry34Ab1/35Ab1.

(12) Inhibitors of mitochondrial ATP synthase, preferably ATP disruptors selected from diafenthiuron, or organotin compounds selected from azocyclotin, cyhexatin and fenbutatin oxide, or propargite or tetradifon.

(13) Uncouplers of oxidative phosphorylation via disruption of the proton gradient selected from chlorfenapyr, DNOC and sulfluramid.

(14) Nicotinic acetylcholine receptor channel blockers selected from bensultap, cartap hydrochloride, thiocyclam, and thiosultap-sodium.

(15) CHS1-related inhibitors of chitin biosynthesis, preferably benzoylureas, selected from bistrifluron, chlorfluazuron, diflubenzuron, flucycloxuron, flufenoxuron, hexaflumuron, lufenuron, novaluron, noviflumuron, teflubenzuron and triflumuron.

(16) Inhibitors of chitin biosynthesis, type 1, selected from buprofezin.

(17) Moulting disruptors (especially in the case of Diptera) selected from cyromazine.

(18) Ecdysone receptor agonists, preferably diacylhydrazines, selected from chromafenozide, halofenozide, methoxyfenozide and tebufenozide.

(19) Octopamine receptor agonists selected from amitraz.

(20) Mitochondrial complex III electron transport inhibitors selected from hydramethylnon, acequinocyl, fluacrypyrim and bifenazate.

(21) Mitochondrial complex I electron transport inhibitors, preferably METI acaricides and insecticides selected from fenazaquin, fenpyroximate, pyrimidifen, pyridaben, tebufenpyrad and tolfenpyrad, or rotenone (Derris).

(22) Blockers of the voltage-gated sodium channel, preferably oxadiazines selected from indoxacarb or semicarbazones selected from metaflumizone.

(23) Inhibitors of acetyl-CoA carboxylase, preferably tetronic and tetramic acid derivatives selected from spirodiclofen, spiromesifen, spiropidion and spirotetramat.

(24) Mitochondrial complex IV electron transport inhibitors, preferably phosphides selected from aluminium phosphide, calcium phosphide, phosphine and zinc phosphide, or cyanides selected from calcium cyanide, potassium cyanide and sodium cyanide.

(25) Mitochondrial complex II electron transport inhibitors, preferably beta-keto nitrile derivatives selected from cyenopyrafen and cyflumetofen, or carboxanilides selected from pyflubumide.

(28) Ryanodine receptor modulators, preferably diamides selected from chlorantraniliprole, cyantraniliprole, cyclaniliprole, flubendiamide and tetraniliprole.

(29) Chordotonal organ modulators (with undefined target structure) selected from flonicamid.

(30) Allosteric modulators of the GABA-gated chloride channel, preferably meta-diamides selected from broflanilide or isoxazoles selected from fluxametamide.

(31) Baculoviruses, preferably granuloviruses (GVs) selected from Cydia pomonella GV and Thaumatotibia leucotreta (GV) or nuclear polyhedrosis viruses (NPVs) selected from Anticarsia gemmatalis MNPV, flucypyriprol and Helicoverpa armigera NPV.

(32) Allosteric modulators (site II) of the nicotinic acetylcholine receptor selected from GS-omega/kappa-HXTX-Hv 1a peptide.

(33) Further active ingredients selected from acynonapyr, afoxolaner, azadirachtin, benclothiaz, benzoximat, benzpyrimoxan, bromopropylate, chinomethionat, chloroprallethrin, cryolite, cyclobutrifluram, cycloxaprid, cyetpyrafen, cyhalodiamide, cyproflanilide (CAS 2375110-88-4), dicloromezotiaz, dicofol, dimpropyridaz, epsilon-metofluthrin, epsilon-momfluthrin, flometoquin, fluazaindolizine, flucypyriprol (CAS 1771741-86-6), fluensulfone, flufenerim, flufenoxystrobin, flufiprole, fluhexafon, fluopyram, flupyrimin, fluralaner, fufenozide, flupentiofenox, guadipyr, heptafluthrin, imidaclothiz, iprodione, isocycloseram, kappa-bifenthrin, kappa-tefluthrin, lotilaner, meperfluthrin, nicofluprole (CAS 1771741-86-6), oxazosulfyl, paichongding, pyridalyl, pyrifluquinazon, pyriminostrobin, sarolaner, spidoxamat, spirobudiclofen, tetramethylfluthrin, tetrachlorantraniliprole, tigolaner, tioxazafen, thiofluoximate, tyclopyrazoflor, iodomethane; additionally preparations based on Bacillus firmus (I-1582, Votivo) and azadirachtin (BioNeem), and the following compounds: 1-{2-fluoro-4-methyl-5-[(2,2,2-trifluoroethyl)sulfinyl]phenyl}-3-(trifluoromethyl)-1H-1,2,4-triazol-5-amine (known from WO2006/043635) (CAS 885026-50-6), 2-chloro-N-[2-{1-[(2E)-3-(4-chlorophenyl)prop-2-en-1-yl]piperidin-4-yl}-4-(trifluoromethyl)phenyl]isonicotinamide (known from WO2006/003494) (CAS 872999-66-1), 3-(4-chloro-2,6-dimethylphenyl)-4-hydroxy-8-methoxy-1,8-diazaspiro[4.5]dec-3-en-2-one (known from WO 2010052161) (CAS 1225292-17-0), 3-(4-chloro-2,6-dimethylphenyl)-8-methoxy-2-oxo-1,8-diazaspiro [4.5]dec-3-en-4-yl ethylcarbonate (known from EP 2647626) (CAS1440516-42-6), PF1364 (known from JP2010/018586) (CAS 1204776-60-2), (3E)-3-[1-[(6-chloro-3-pyridyl)methyl]-2-pyridylidene]-1,1,1-trifluoropropan-2-one (known from WO2013/144213) (CAS 1461743-15-6), N-[3-(benzylcarbamoyl)-4-chlorophenyl]-1-methyl-3-(pentafluoroethyl)-4-(trifluoromethyl)-1H-pyrazole-5-carboxamide (known from WO2010/051926) (CAS 1226889-14-0), 5-bromo-4-chloro-N-[4-chloro-2-methyl-6-(methylcarbamoyl)phenyl]-2-(3-chloro-2-pyridyl)pyrazole-3-carboxamide (known from CN103232431) (CAS 1449220-44-3), 4-[5-(3,5-dichlorophenyl)-4,5-dihydro-5-(trifluoromethyl)-3-isoxazolyl]-2-methyl-N-(cis-1-oxido-3-thietanyl)benzamide, 4-[5-(3,5-dichlorophenyl)-4,5-dihydro-5-(trifluoromethyl)-3-isoxazolyl]-2-methyl-N-(trans-1-oxido-3-thietanyl)benzamide and 4-[(5S)-5-(3,5-dichlorophenyl)-4,5-dihydro-5-(trifluoromethyl)-3-isoxazolyl]-2-methyl-N-(cis-1-oxido-3-thietanyl)benzamide (known from WO 2013/050317 A1) (CAS 1332628-83-7), N-[3-chloro-1-(3-pyridinyl)-1H-pyrazol-4-yl]-N-ethyl-3-[(3,3,3-trifluoropropyl)sulfinyl]propanamide, (+)-N-[3-chloro-1-(3-pyridinyl)-1H-pyrazol-4-yl]-N-ethyl-3-[(3,3,3-trifluoropropyl)sulfinyl]propanamide and (−)-N-[3-chloro-1-(3-pyridinyl)-1H-pyrazol-4-yl]-N-ethyl-3-[(3,3,3-trifluoropropyl)sulfinyl]propanamide (known from WO 2013/162715 A2, WO 2013/162716 A2, US 2014/0213448 A1) (CAS 1477923-37-7), 5-[[(2E)-3-chloro-2-propen-1-yl]amino]-1-[2,6-dichloro-4-(trifluoromethyl)phenyl]-4-[(trifluoromethyl)sulfinyl]-1H-pyrazole-3-carbonitrile (known from CN 101337937 A) (CAS 1105672-77-2), 3-bromo-N-[4-chloro-2-methyl-6-[(methylamino)thioxomethyl]phenyl]-1-(3-chloro-2-pyridinyl)-1H-pyrazole-5-carboxamide, (Liudaibenjiaxuanan, known from CN 103109816 A) (CAS 1232543-85-9); N-[4-chloro-2-[[(1,1-dimethylethyl)amino]carbonyl]-6-methylphenyl]-1-(3-chloro-2-pyridinyl)-3-(fluoromethoxy)-1H-pyrazole-5-carboxamide (known from WO 2012/034403 A1) (CAS 1268277-22-0), N-[2-(5-amino-1,3,4-thiadiazol-2-yl)-4-chloro-6-methylphenyl]-3-bromo-1-(3-chloro-2-pyridinyl)-1H-pyrazole-5-carboxamide (known from WO 2011/085575 A1) (CAS 1233882-22-8), 4-[3-[2,6-dichloro-4-[(3,3-dichloro-2-propen-1-yl)oxy]phenoxy]propoxy]-2-methoxy-6-(trifluoromethyl)pyrimidine (known from CN 101337940 A) (CAS 1108184-52-6); (2E)- and 2(Z)-2-[2-(4-cyanophenyl)-1-[3-(trifluoromethyl)phenyl]ethylidene]-N-[4-(difluoromethoxy)phenyl]hydrazinecarboxamide (known from CN 101715774 A) (CAS 1232543-85-9); 3-(2,2-dichloroethenyl)-2,2-dimethyl-4-(1H-benzimidazol-2-yl)phenyl cyclopropanecarboxylate (known from CN 103524422 A) (CAS 1542271-46-4); methyl (4aS)-7-chloro-2,5-dihydro-2-[[(methoxycarbonyl)[4-[(trifluoromethyl)thio]phenyl]amino]carbonyl]indeno [1,2-e][1,3,4]oxadiazine-4a(3H)-carboxylate (known from CN 102391261 A) (CAS 1370358-69-2); 6-desoxy-3-O-ethyl-2,4-di-O-methyl-1-[N-[4-[1-[4-(1,1,2,2,2-pentafluoroethoxy)phenyl]-1H-1,2,4-triazol-3-yl]phenyl]carbamat]-α-L-mannopyranose (known from US 2014/0275503 A1) (CAS 1181213-14-8); 8-(2-cyclopropylmethoxy-4-trifluoromethylphenoxy)-3-(6-trifluoromethylpyridazin-3-yl)-3-azabicyclo[3.2.1]octane (CAS 1253850-56-4), (8-anti)-8-(2-cyclopropylmethoxy-4-trifluoromethylphenoxy)-3-(6-trifluoromethylpyridazin-3-yl)-3-azabicyclo[3.2.1]octane (CAS 933798-27-7), (8-syn)-8-(2-cyclopropylmethoxy-4-trifluoromethylphenoxy)-3-(6-trifluoromethylpyridazin-3-yl)-3-azabicyclo[3.2.1]octane (known from WO 2007040280 A1, WO 2007040282 A1) (CAS 934001-66-8), N-[4-(aminothioxomethyl)-2-methyl-6-[(methylamino)carbonyl]phenyl]-3-bromo-1-(3-chloro-2-pyridinyl)-1H-pyrazole-5-carboxamide (known from CN 103265527 A) (CAS 1452877-50-7), 3-(4-chloro-2,6-dimethylphenyl)-8-methoxy-1-methyl-1,8-diazaspiro[4.5]decane-2,4-dione (known from WO 2014/187846 A1) (CAS 1638765-58-8), ethyl 3-(4-chloro-2,6-dimethylphenyl)-8-methoxy-1-methyl-2-oxo-1,8-diazaspiro[4.5]dec-3-en-4-ylcarboxylate (known from WO 2010/066780 A1, WO 2011151146 A1) (CAS 1229023-00-0), N-[11-(2,6-difluorophenyl)-1H-pyrazol-3-yl]-2-(trifluoromethyl)benzamide (known from WO 2014/053450 A1) (CAS 1594624-87-9), N-[2-(2,6-difluorophenyl)-2H-1,2,3-triazol-4-yl]-2-(trifluoromethyl)benzamide (known from WO 2014/053450 A1) (CAS 1594637-65-6), N-[1-(3,5-difluoro-2-pyridinyl)-1H-pyrazol-3-yl]-2-(trifluoromethyl)benzamide (known from WO 2014/053450 A1) (CAS 1594626-19-3), (3R)-3-(2-chloro-5-thiazolyl)-2,3-dihydro-8-methyl-5,7-dioxo-6-phenyl-5H-thiazolo[3,2-a]pyrimidinium internal salt (known from WO 2018/177970 A1) (CAS 2246757-58-2); 3-(2-chloro-5-thiazolyl)-2,3-dihydro-8-methyl-5,7-dioxo-6-phenyl-5H-thiazolo[3,2-a]pyrimidinium internal salt (known from WO 2018/177970 A1) (CAS 2246757-56-0); N-[3-chloro-1-(3-pyridinyl)-1H-pyrazol-4-yl]-2-(methylsulfonyl)propanamide (known from WO 2019/236274 A1) (CAS 2396747-83-2), N-[2-bromo-4-[1,2,2,2-tetrafluoro-1-(trifluoromethyl)ethyl]-6-(trifluoromethyl)phenyl]-2-fluoro-3-[(4-fluorobenzoyl)amino]benzamide (known from WO 2019059412 A1) (CAS 1207977-87-4), 3-bromo-1-(3-chloro-2-pyridinyl)-N-[4,6-dichloro-3-fluoro-2-[(methylamino)carbonyl]phenyl]-1H-pyrazole-5-carboxamide (fluchlorodiamide; known from CN110835330 A, CN106977494 A) (CAS: 2129147-03-9).

### Nematicides

The active ingredients identified here by their common names are known and are described, for example, in the pesticides handbook (“The Pesticide Manual”, 16th ed., British Crop Protection Council 2012) or can be found on the Internet (e.g. http://www.alanwood.net/pesticides). The classification is based on the IRAC Mode of Action Classification Groups current at the filing date of the present patent application.

(Group N-1) Acetylcholinesterase (AChE) inhibitors, preferably (N-1A) carbamates selected from aldicarb, benfuracarb, carbofuran, carbosulfan and thiodicarb, or (N-1B) organophosphates selected from cadusafos, ethoprofos, fenamiphos, fosthiazat, imicyafos, phorate and terbufos.

(Group N-2) Glutamate-gated chloride channel (GluCl) allosteric modulators, preferably avermectins selected from abamectin and emamectin benzoate.

(Group N-3) Mitochondrial complex II electron transport inhibitors, especially inhibitors of succinate coenzyme Q reductase, preferably pyridinylmethylbenzamides selected from fluopyram.

(Group N-4) Lipid synthesis/growth regulation modulators, especially inhibitors of acetyl-CoA carboxylase, preferably tetronic acid and tetramic acid derivatives selected from spirotetramat.

(Group N-UN) Compounds of unknown or uncertain mode of action and various chemistries, selected from fluensulfone, fluazaindolizine, furfural, iprodione and tioxazafen.

(Group N-UNX) Compounds of uncertain or unknown mode of action: Suspected multisite inhibitors, preferably volatile sulfur-producing compounds selected from carbon disulfide and dimethyl disulfide (DMDS), or carbon disulfide-releasing compounds selected from sodium tetrathiocarbonate, or alkyl halides selected from methyl bromide and methyl iodide (iodomethane), or halogenated hydrocarbons selected from 1,2-dibromo-3-chloropropane (DBCP) and 1,3-dichloropropene, or chloropicrin, or methyl isothiocyanate-producing compounds selected from allyl isothiocyanate, diazomet, metam-potassium and metam-sodium.

(Group N-UNB) Bacterial agents (non-Bt) of unknown or uncertain mode of action, preferably a bacterium or obtained from a bacterium selected from Burkholderia spp., e.g. rinojensis A396, Bacillus spp., e.g. firmus, licheniformis, amyloliquefaciens or subtilis, Pasteuria spp., e.g. penetrans or nishizawae, Pseudomonas spp., e.g. chlororaphis or fluorescens, and Streptomyces spp., e.g. lydicus, dicklowii or albogriseolus.

(Group N-UNF) Fungal agents of unknown or uncertain mode of action, preferably a fungus or obtained from a fungus selected from Actinomyces spp., e.g. streptococcus, Arthrobotrys spp., e.g. oligospora, Aspergillus spp., e.g. niger, Muscodor spp., e.g. albus, Myrothecium spp., e.g. verrucaria, Paecilomyces spp., e.g. lilacinus (Purpureocillium lilacinum), carneus or fumosoroseus, Pochonia spp., e.g. chlamydosporia, and Trichoderma spp., e.g. harzianum, virens, atroviride or viride.

(Group N-UNE) Botanic agents or agents originating from animals, including synthetic extracts and unrefined oils, of uncertain or unknown mode of action, preferably botanic agents or agents originating from animals selected from azadirachtin, Camellia seed cake, essential oils, garlic extract, pongamia oil, terpenes, e.g. carvacrol, and Quillaja saponaria extract.

### Fungicides

The active ingredients specified here by their common names are known and are described, for example, in the “Pesticide Manual”, (16th ed., British Crop Protection Council) or can be searched for on the Internet (e.g. www.alanwood.net/pesticides).

All the mixing partners mentioned in classes (1) to (15), as the case may be, may form salts with suitable bases or acids if they are capable of doing so on the basis of their functional groups. All the fungicidal mixing partners mentioned in classes (1) to (15), as the case may be, may include tautomeric forms.

1) Inhibitors of ergosterol biosynthesis, for example (1.001) cyproconazole, (1.002) difenoconazole, (1.003) epoxiconazole, (1.004) fenbuconazole, (1.005) fenhexamid, (1.006) fenpropidin, (1.007) fenpropimorph, (1.008) fenpyrazamin, (1.009) fluquinconazole, (1.010) flutriafol, (1.011) hexaconazole, (1.012) imazalil, (1.013) imazalil sulfate, (1.014) ipconazole, (1.015) ipfentrifluconazole, (1.016) mefentrifluconazole, (1.017) metconazole, (1.018) myclobutanil, (1.019) paclobutrazole, (1.020) penconazole, (1.021) prochloraz, (1.022) propiconazole, (1.023) prothioconazole, (1.024) pyrisoxazole, (1.025) spiroxamine, (1.026) tebuconazole, (1.027) tetraconazole, (1.028) triadimenol, (1.029) tridemorph, (1.030) triticonazole, (1.031) (1R,2S,5S)-5-(4-chlorobenzyl)-2-(chloromethyl)-2-methyl-1-(1H-1,2,4-triazol-1-ylmethyl)cyclopentanol, (1.032) (1S,2R,5R)-5-(4-chlorobenzyl)-2-(chloromethyl)-2-methyl-1-(1H-1,2,4-triazol-1-ylmethyl)cyclopentanol, (1.033) (2R)-2-(1-chlorocyclopropyl)-4-[(1R)-2,2-dichlorocyclopropyl]-1-(1H-1,2,4-triazol-1-yl)butan-2-ol, (1.034) (2R)-2-(1-chlorocyclopropyl)-4-[(1S)-2,2-dichlorocyclopropyl]-1-(1H-1,2,4-triazol-1-yl)butan-2-ol, (1.035) (2R)-2-[4-(4-chlorophenoxy)-2-(trifluoromethyl)phenyl]-1-(1H-1,2,4-triazol-1-yl)propan-2-ol, (1.036) (2S)-2-(1-chlorocyclopropyl)-4-[(1R)-2,2-dichlorocyclopropyl]-1-(1H-1,2,4-triazol-1-yl)butan-2-ol, (1.037) (2S)-2-(1-chlorocyclopropyl)-4-[(1S)-2,2-dichlorocyclopropyl]-1-(1H-1,2,4-triazol-1-yl)butan-2-ol, (1.038) (2S)-2-[4-(4-chlorophenoxy)-2-(trifluoromethyl)phenyl]-1-(1H-1,2,4-triazol-1-yl)propan-2-ol, (1.039) (R)-[3-(4-chloro-2-fluorophenyl)-5-(2,4-difluorophenyl)-1,2-oxazol-4-yl](pyridin-3-yl)methanol, (1.040) (S)-[3-(4-chloro-2-fluorophenyl)-5-(2,4-difluorophenyl)-1,2-oxazol-4-yl](pyridin-3-yl)methanol, (1.041) [3-(4-chloro-2-fluorophenyl)-5-(2,4-difluorophenyl)-1,2-oxazol-4-yl](pyridin-3-yl)methanol, (1.042) 1-({(2R,4S)-2-[2-chloro-4-(4-chlorophenoxy)phenyl]-4-methyl-1,3-dioxolan-2-yl}methyl)-1H-1,2,4-triazole, (1.043) 1-({(2S,4S)-2-[2-chloro-4-(4-chlorophenoxy)phenyl]-4-methyl-1,3-dioxolan-2-yl}methyl)-1H-1,2,4-triazole, (1.044) 1-{1[3-(2-chlorophenyl)-2-(2,4-difluorophenyl)oxiran-2-yl]methyl}-1H-1,2,4-triazol-5-yl thiocyanate, (1.045) 1-{[rel(2R,3R)-3-(2-chlorophenyl)-2-(2,4-difluorophenyl)oxiran-2-yl]methyl}-1H-1,2,4-triazol-5-yl thiocyanate, (1.046) 1-{[rel(2R,3S)-3-(2-chlorophenyl)-2-(2,4-difluorophenyl)oxiran-2-yl]methyl}-1H-1,2,4-triazol-5-yl thiocyanate, (1.047) 2-[(2R,4R,5R)-1-(2,4-dichlorophenyl)-5-hydroxy-2,6,6-trimethylheptan-4-yl]-2,4-dihydro-3H-1,2,4-triazole-3-thione, (1.048) 2-[(2R,4R,5S)-1-(2,4-dichlorophenyl)-5-hydroxy-2,6,6-trimethylheptan-4-yl]-2,4-dihydro-3H-1,2,4-triazole-3-thione, (1.049) 2-[(2R,4S,5R)-1-(2,4-dichlorophenyl)-5-hydroxy-2,6,6-trimethylheptan-4-yl]-2,4-dihydro-3H-1,2,4-triazole-3-thione, (1.050) 2-[(2R,4S,5S)-1-(2,4-dichlorophenyl)-5-hydroxy-2,6,6-trimethylheptan-4-yl]-2,4-dihydro-3H-1,2,4-triazole-3-thione, (1.051) 2-[(2S,4R,5R)-1-(2,4-dichlorophenyl)-5-hydroxy-2,6,6-trimethylheptan-4-yl]-2,4-dihydro-3H-1,2,4-triazole-3-thione, (1.052) 2-[(2S,4R,5S)-1-(2,4-dichlorophenyl)-5-hydroxy-2,6,6-trimethylheptan-4-yl]-2,4-dihydro-3H-1,2,4-triazole-3-thione, (1.053) 2-[(2S,4S,5R)-1-(2,4-dichlorophenyl)-5-hydroxy-2,6,6-trimethylheptan-4-yl]-2,4-dihydro-3H-1,2,4-triazole-3-thione, (1.054) 2-[(2S,4S,5S)-1-(2,4-dichlorophenyl)-5-hydroxy-2,6,6-trimethylheptan-4-yl]-2,4-dihydro-3H-1,2,4-triazole-3-thione, (1.055) 2-[1-(2,4-dichlorophenyl)-5-hydroxy-2,6,6-trimethylheptan-4-yl]-2,4-dihydro-3H-1,2,4-triazole-3-thione, (1.056) 2-[6-(4-bromophenoxy)-2-(trifluoromethyl)-3-pyridyl]-1-(1,2,4-triazol-1-yl)propan-2-ol, (1.057) 2-[6-(4-chlorophenoxy)-2-(trifluoromethyl)-3-pyridyl]-1-(1,2,4-triazol-1-yl)propan-2-ol, (1.058) 2-{[3-(2-chlorophenyl)-2-(2,4-difluorophenyl)oxiran-2-yl]methyl}-2,4-dihydro-3H-1,2,4-triazole-3-thione, (1.059) 2-{[rel(2R,3R)-3-(2-chlorophenyl)-2-(2,4-difluorophenyl)oxiran-2-yl]methyl}-2,4-dihydro-3H-1,2,4-triazole-3-thione, (1.060) 2-{[rel(2R,3S)-3-(2-chlorophenyl)-2-(2,4-difluorophenyl)-oxiran-2-yl]methyl}-2,4-dihydro-3H-1,2,4-triazole-3-thione, (1.061) 3-[2-(1-chlorocyclopropyl)-3-(3-chloro-2-fluorophenyl)-2-hydroxypropyl]imidazole-4-carbonitrile, (1.062) 4-[[6-[rac-(2R)-2-(2,4-difluorophenyl)-1,1-difluoro-2-hydroxy-3-(5-thioxo-4H-1,2,4-triazol-1-yl)propyl]-3-pyridyl]oxy]benzonitrile, (1.063) 5-(4-chlorobenzyl)-2-(chloromethyl)-2-methyl-1-(1H-1,2,4-triazol-1-ylmethyl)cyclopentanol, (1.064) 5-(allylsulfanyl)-1-{[3-(2-chlorophenyl)-2-(2,4-difluorophenyl)oxiran-2-yl]methyl}-1H-1,2,4-triazole, (1.065) 5-(allylsulfanyl)-1-{[rel(2R,3R)-3-(2-chlorophenyl)-2-(2,4-difluorophenyl)oxiran-2-yl]methyl}-1H-1,2,4-triazole, (1.066) 5-(allylsulfanyl)-1-{[rel(2R,3S)-3-(2-chlorophenyl)-2-(2,4-difluorophenyl)oxiran-2-yl]methyl}-1H-1,2,4-triazole, (1.067) methyl 2-[2-chloro-4-(4-chlorophenoxy)phenyl]-2-hydroxy-3-(1H-1,2,4-triazol-1-yl)propanoate, (1.068) N′-(2-chloro-5-methyl-4-phenoxyphenyl)-N-ethyl-N-methylimidoformamide, (1.069) N′-[2-chloro-4-(2-fluorophenoxy)-5-methylphenyl]-N-ethyl-N-methylimidoformamide, (1.070) N′-[5-bromo-6-(2,3-dihydro-1H-inden-2-yloxy)-2-methylpyridin-3-yl]-N-ethyl-N-methylimidoformamide, (1.071) N′-{4-[(4,5-dichloro-1,3-thiazol-2-yl)oxy]-2,5-dimethylphenyl}-N-ethyl-N-methylimidoformamide, (1.072) N′-{5-bromo-2-methyl-6-[(1-propoxypropan-2-yl)oxy]pyridin-3-yl}-N-ethyl-N-methylimidoformamide, (1.073) N′-{5-bromo-6-[(1R)-1-(3,5-difluorophenyl)ethoxy]-2-methylpyridin-3-yl}-N-ethyl-N-methylimidoformamide, (1.074) N′-{5-bromo-6-[(1S)-1-(3,5-difluorophenyl)ethoxy]-2-methylpyridin-3-yl}-N-ethyl-N-methylimidoformamide, (1.075) N′-{5-bromo-6-[(cis-4-isopropylcyclohexyl)oxy]-2-methylpyridin-3-yl}-N-ethyl-N-methylimidoformamide, (1.076) N′-{5-bromo-6-[(trans-4-isopropylcyclohexyl)oxy]-2-methylpyridin-3-yl}-N-ethyl-N-methylimidoformamide, (1.077) N′-{5-bromo-6-[1-(3,5-difluorophenyl)ethoxy]-2-methylpyridin-3-yl}-N-ethyl-N-methylimidoformamide, (1.078)N-isopropyl-N′-[5-methoxy-2-methyl-4-(2,2,2-trifluoro-1-hydroxy-1-phenylethyl)phenyl]-N-methylimidoformamide.

2) Respiratory chain inhibitors acting on complex I oder II, for example (2.001) benzovindiflupyr, (2.002) bixafen, (2.003) boscalid, (2.004) carboxin, (2.005) cyclobutrifluram, (2.006) flubeneteram, (2.007) fluindapyr, (2.008) fluopyram, (2.009) flutolanil, (2.010) fluxapyroxad, (2.011) furametpyr, (2.012) inpyrfluxam, (2.013) isofetamid, (2.014) isoflucypram, (2.015) isopyrazam, (2.016) penflufen, (2.017) penthiopyrad, (2.018) pydiflumetofen, (2.019) pyrapropoyn, (2.020) pyraziflumid, (2.021) sedaxan, (2.022) 1,3-dimethyl-N-(1,1,3-trimethyl-2,3-dihydro-1H-inden-4-yl)-1H-pyrazole-4-carboxamide, (2.023) 1,3-dimethyl-N-[(3R)-1,1,3-trimethyl-2,3-dihydro-1H-inden-4-yl]-1H-pyrazole-4-carboxamide, (2.024) 1,3-dimethyl-N-[(3S)-1,1,3-trimethyl-2,3-dihydro-1H-inden-4-yl]-1H-pyrazole-4-carboxamide, (2.025) 1-methyl-3-(trifluoromethyl)-N-[2′-(trifluoromethyl)biphenyl-2-yl]-1H-pyrazole-4-carboxamide, (2.026) 2-fluoro-6-(trifluoromethyl)-N-(1,1,3-trimethyl-2,3-dihydro-1H-inden-4-yl)benzamide, (2.027) 3-(difluoromethyl)-1-methyl-N-(1,1,3-trimethyl-2,3-dihydro-1H-inden-4-yl)-1H-pyrazole-4-carboxamide, (2.028) 3-(difluoromethyl)-1-methyl-N-[(3S)-1,1,3-trimethyl-2,3-dihydro-1H-inden-4-yl]-1H-pyrazole-4-carboxamide, (2.029) 3-(difluoromethyl)-N-[(3R)-7-fluoro-1,1,3-trimethyl-2,3-dihydro-1H-inden-4-yl]-1-methyl-1H-pyrazole-4-carboxamide, (2.030) 3-(difluoromethyl)-N-[(3S)-7-fluoro-1,1,3-trimethyl-2,3-dihydro-1H-inden-4-yl]-1-methyl-1H-pyrazole-4-carboxamide, (2.031) 5,8-difluoro-N-[2-(2-fluoro-4-{[4-(trifluoromethyl)pyridin-2-yl]oxy}phenyl)ethyl]quinazoline-4-amine, (2.032)N-[((1R,4S)-9-(dichloromethylene)-1,2,3,4-tetrahydro-1,4-methanonaphthalen-5-yl]-3-(difluoromethyl)-1-methyl-1H-pyrazole-4-carboxamide, (2.033)N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide, (2.034)N-[1-(2,4-dichlorophenyl)-1-methoxypropan-2-yl]-3-(difluoromethyl)-1-methyl-1H-pyrazole-4-carboxamide, (2.035)N-[rac-(1S,2S)-2-(2,4-dichlorophenyl)cyclobutyl]-2-(trifluoromethyl)nicotinamide.

3) Respiratory chain inhibitors acting on complex III, for example (3.001) ametoctradin, (3.002) amisulbrom, (3.003) azoxystrobin, (3.004) coumethoxystrobin, (3.005) coumoxystrobin, (3.006) cyazofamid, (3.007) dimoxystrobin, (3.008) enoxastrobin, (3.009) famoxadone, (3.010) fenamidone, (3.011) fenpicoxamid, (3.012) florylpicoxamid, (3.013) flufenoxystrobin, (3.014) fluoxastrobin, (3.015) kresoxim-methyl, (3.016) mandestrobin, (3.017) metominostrobin, (3.018) metyltetraprole, (3.019) orysastrobin, (3.020) picoxystrobin, (3.021) pyraclostrobin, (3.022) pyrametostrobin, (3.023) pyraoxystrobin, (3.024) trifloxystrobin, (3.025) (2E)-2-{2-[({f[(1E)-1-(3-{f[(E)-1-fluoro-2-phenylvinyl]oxy}phenyl)ethylidene]amino}oxy)methyl]phenyl}-2-(methoxyimino)-N-methylacetamide, (3.026) (2E,3Z)-5-{[1-(4-chlorophenyl)-1H-pyrazol-3-yl]oxy}-2-(methoxyimino)-N,3-dimethylpent-3-enamide, (3.027) (2R)-2-{2-[(2,5-dimethylphenoxy)methyl]phenyl}-2-methoxy-N-methylacetamide, (3.028) (2S)-2-{2-[(2,5-dimethylphenoxy)methyl]phenyl}-2-methoxy-N-methylacetamide, (3.029) N-(3-ethyl-3,5,5-trimethylcyclohexyl)-3-formamido-2-hydroxybenzamide, (3.030) (2E,3Z)-5-{[1-(4-chloro-2-fluorophenyl)-1H-pyrazol-3-yl]oxy}-2-(methoxyimino)-N,3-dimethylpent-3-enamide, (3.031) methyl {5-[3-(2,4-dimethylphenyl)-1H-pyrazol-1-yl]-2-methylbenzyl}carbamate.

4) Mitosis and cell division inhibitors, for example (4.001) carbendazim, (4.002) diethofencarb, (4.003) ethaboxam, (4.004) fluopicolid, (4.005) fluopimomid, (4.006) metrafenone, (4.007) pencycuron, (4.008) pyridachlometyl, (4.009) pyriofenon (chlazafenon), (4.010) thiabendazole, (4.011) thiophanat-methyl, (4.012) zoxamide, (4.013) 3-chloro-5-(4-chlorophenyl)-4-(2,6-difluorophenyl)-6-methylpyridazine, (4.014) 3-chloro-5-(6-chloropyridin-3-yl)-6-methyl-4-(2,4,6-trifluorophenyl)pyridazine, (4.015) 4-(2-bromo-4-fluorophenyl)-N-(2,6-difluorophenyl)-1,3-dimethyl-1H-pyrazole-5-amine, (4.016) 4-(2-bromo-4-fluorophenyl)-N-(2-bromo-6-fluorophenyl)-1,3-dimethyl-1H-pyrazole-5-amine, (4.017) 4-(2-bromo-4-fluorophenyl)-N-(2-bromophenyl)-1,3-dimethyl-1H-pyrazole-5-amine, (4.018) 4-(2-bromo-4-fluorophenyl)-N-(2-chloro-6-fluorophenyl)-1,3-dimethyl-1H-pyrazole-5-amine, (4.019) 4-(2-bromo-4-fluorophenyl)-N-(2-chlorophenyl)-1,3-dimethyl-1H-pyrazole-5-amine, (4.020) 4-(2-bromo-4-fluorophenyl)-N-(2-fluorophenyl)-1,3-dimethyl-1H-pyrazole-5-amine, (4.021) 4-(2-chloro-4-fluorophenyl)-N-(2,6-difluorophenyl)-1,3-dimethyl-1H-pyrazole-5-amine, (4.022) 4-(2-chloro-4-fluorophenyl)-N-(2-chloro-6-fluorophenyl)-1,3-dimethyl-1H-pyrazole-5-amine, (4.023) 4-(2-chloro-4-fluorophenyl)-N-(2-chlorophenyl)-1,3-dimethyl-1H-pyrazole-5-amine, (4.024) 4-(2-chloro-4-fluorophenyl)-N-(2-fluorophenyl)-1,3-dimethyl-1H-pyrazole-5-amine, (4.025) 4-(4-chlorophenyl)-5-(2,6-difluorophenyl)-3,6-dimethylpyridazine, (4.026)N-(2-bromo-6-fluorophenyl)-4-(2-chloro-4-fluorophenyl)-1,3-dimethyl-1H-pyrazole-5-amine, (4.027)N-(2-bromophenyl)-4-(2-chloro-4-fluorophenyl)-1,3-dimethyl-1H-pyrazole-5-amine, (4.028)N-(4-chloro-2,6-difluorophenyl)-4-(2-chloro-4-fluorophenyl)-1,3-dimethyl-1H-pyrazole-5-amine.

5) Compounds that can show multisite action, for example (5.001) Bordeaux mixture, (5.002) captafol, (5.003) captan, (5.004) chlorthalonil, (5.005) copper hydroxide, (5.006) copper naphthenate, (5.007) copper oxide, (5.008) copper oxychloride, (5.009) copper(2+) sulfate, (5.010) dithianon, (5.011) dodine, (5.012) folpet, (5.013) mancozeb, (5.014) maneb, (5.015) metiram, (5.016) zinc metiram, (5.017) copper oxine, (5.018) propineb, (5.019) sulfur and sulfur preparations including calcium polysulfide, (5.020) thiram, (5.021) zineb, (5.022) ziram, (5.023) 6-ethyl-5,7-dioxo-6,7-dihydro-5H-pyrrolo[3′,4′:5,6][1,4]dithiino[2,3-c][1,2]thiazole-3-carbonitrile.

6) Compounds capable of inducing host defence reactions, for example (6.001) acibenzolar-S-methyl, (6.002) fosetyl-aluminium, (6.003) fosetyl-calcium, (6.004) fosetyl-sodium, (6.005) isotianil, (6.006) phosphoric acid and salts thereof, (6.007) probenazole, (6.008) tiadinil.

7) Amino acid and/or protein biosynthesis inhibitors, for example (7.001) cyprodinil, (7.002) kasugamycin, (7.003) kasugamycin hydrochloride hydrate, (7.004) oxytetracycline, (7.005) pyrimethanil.

8) ATP production inhibitors, for example (8.001) silthiofam.

9) Cell wall synthesis inhibitors, for example (9.001) benthiavalicarb, (9.002) dimethomorph, (9.003) flumorph, (9.004) iprovalicarb, (9.005) mandipropamid, (9.006) pyrimorph, (9.007) valifenalate, (9.008) (2E)-3-(4-tert-butylphenyl)-3-(2-chloropyridin-4-yl)-1-(morpholin-4-yl)prop-2-en-1-one, (9.009) (2Z)-3-(4-tert-butylphenyl)-3-(2-chloropyridin-4-yl)-1-(morpholin-4-yl)prop-2-en-1-one.

10) Lipid synthesis or transport inhibitors or membrane synthesis inhibitors, for example (10.001) fluoxapiprolin, (10.002) natamycin, (10.003) oxathiapiprolin, (10.004) propamocarb, (10.005) propamocarb hydrochloride, (10.006) propamocarb-fosetylate, (10.007) tolclofos-methyl, (10.008) 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (10.009) 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (10.010) 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone, (10.011) 2-[3,5-bis(difluoromethyl)-1H-pyrazol-1-yl]-1-[4-(4-{5-[2-chloro-6-(prop-2-yn-1-yloxy)phenyl]-4,5-dihydro-1,2-oxazol-3-yl}-1,3-thiazol-2-yl)piperidin-1-yl]ethanone, (10.012) 2-[3,5-bis(difluoromethyl)-1H-pyrazol-1-yl]-1-[4-(4-{5-[2-fluoro-6-(prop-2-yn-1-yloxy)phenyl]-4,5-dihydro-1,2-oxazol-3-yl}-1,3-thiazol-2-yl)piperidin-1-yl]ethanone, (10.013) 2-{(5R)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (10.014) 2-{(5S)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (10.015) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-phenyl methanesulfonate, (10.016) 3-[2-(1-{[5-methyl-3-(trifluoromethyl)-1H-pyrazol-1-yl]acetyl}piperidin-4-yl)-1,3-thiazol-4-yl]-1,5-dihydro-2,4-benzodioxepin-6-yl methanesulfonate, (10.017) 9-fluoro-3-[2-(1-{[5-methyl-3-(trifluoromethyl)-1H-pyrazol-1-yl]acetyl}piperidin-4-yl)-1,3-thiazol-4-yl]-1,5-dihydro-2,4-benzodioxepin-6-yl methanesulfonate, (10.018) 3-[2-(1-{1[3,5-bis(difluoromethyl)-1H-pyrazol-1-yl]acetyl}piperidin-4-yl)-1,3-thiazol-4-yl]-1,5-dihydro-2,4-benzodioxepin-6-yl methanesulfonate, (10.019) 3-[2-(1-{1[3,5-bis(difluoromethyl)-1H-pyrazol-1-yl]acetyl}piperidin-4-yl)-1,3-thiazol-4-yl]-9-fluoro-1,5-dihydro-2,4-benzodioxepin-6-yl methanesulfonate.

11) Melanin biosynthesis inhibitors, for example (11.001) tolprocarb, (11.002) tricyclazole.

12) Nucleic acid synthesis inhibitors, for example (12.001) benalaxyl, (12.002) benalaxyl-M (kiralaxyl), (12.003) metalaxyl, (12.004) metalaxyl-M (mefenoxam).

13) Signal transduction inhibitors, for example (13.001) fludioxonil, (13.002) iprodione, (13.003) procymidone, (13.004) proquinazid, (13.005) quinoxyfen, (13.006) vinclozolin.

14) Compounds that can act as uncouplers, for example (14.001) fluazinam, (14.002) meptyldinocap.

15) Further compounds, for example (15.001) abscisic acid, (15.002) aminopyrifen, (15.003) benthiazole, (15.004) bethoxazin, (15.005) capsimycin, (15.006) carvone, (15.007) chinomethionat, (15.008) cufraneb, (15.009) cyflufenamid, (15.010) cymoxanil, (15.011) cyprosulfamide, (15.012) dipymetitron, (15.013) flutianil, (15.014) ipflufenoquin, (15.015) methyl isothiocyanate, (15.016) mildiomycin, (15.017) nickel dimethyldithiocarbamate, (15.018) nitrothal-isopropyl, (15.019) oxyfenthiin, (15.020) pentachlorophenol and salts, (15.021) picarbutrazox, (15.022) quinofumelin, (15.023) D-tagatose, (15.024) tebufloquin, (15.025) tecloftalam, (15.026) tolnifanid, (15.027) 2-(6-benzylpyridin-2-yl)quinazoline, (15.028) 2-[6-(3-fluoro-4-methoxyphenyl)-5-methylpyridin-2-yl]quinazoline, (15.029) 2-phenylphenol and salts, (15.030) 4-amino-5-fluoropyrimidin-2-ol (tautomeric form: 4-amino-5-fluoropyrimidin-2(1H)-one), (15.031) 4-oxo-4-[(2-phenylethyl)amino]butanoic acid, (15.032) 5-amino-1,3,4-thiadiazole-2-thiol, (15.033) 5-chloro-N′-phenyl-N′-(prop-2-yn-1-yl)thiophene-2-sulfonohydrazide, (15.034) 5-fluoro-2-[(4-fluorobenzyl)oxy]pyrimidine-4-amine, (15.035) 5-fluoro-2-[(4-methylbenzyl)oxy]pyrimidine-4-amine, (15.036) 5-fluoro-4-imino-3-methyl-1-[(4-methylphenyl)sulfonyl]-3,4-dihydropyrimidin-2(1H)-one, (15.037) but-3-yn-1-yl {6-[({[(Z)-(1-methyl-1H-tetrazol-5-yl)(phenyl)methylene]amino}oxy)methyl]pyridin-2-yl}carbamate, (15.038) ethyl (2Z)-3-amino-2-cyano-3-phenylacrylate, (15.039) phenazine-1-carboxylic acid, (15.040) propyl 3,4,5-trihydroxybenzoate, (15.041) quinolin-8-ol, (15.042) quinolin-8-ol sulfate (2:1), (15.043) 1-(4,5-dimethyl-1H-benzimidazol-1-yl)-4,4-difluoro-3,3-dimethyl-3,4-dihydroisoquinoline, (15.044) 1-(5-(fluoromethyl)-6-methylpyridin-3-yl)-4,4-difluoro-3,3-dimethyl-3,4-dihydroisoquinoline, (15.045) 1-(5,6-dimethylpyridin-3-yl)-4,4-difluoro-3,3-dimethyl-3,4-dihydroisoquinoline, (15.046) 1-(6-(difluoromethyl)-5-methoxypyridin-3-yl)-4,4-difluoro-3,3-dimethyl-3,4-dihydroisoquinoline, (15.047) 1-(6-(difluoromethyl)-5-methylpyridin-3-yl)-4,4-difluoro-3,3-dimethyl-3,4-dihydroisoquinoline, (15.048) 1-(6,7-dimethylpyrazolo[1,5-a]pyridin-3-yl)-4,4-difluoro-3,3-dimethyl-3,4-dihydroisoquinoline, (15.049) 2-{2-fluoro-6-[(8-fluoro-2-methylquinolin-3-yl)oxy]phenyl}propan-2-ol, (15.050) 3-(4,4,5-trifluoro-3,3-dimethyl-3,4-dihydroisoquinoline-1-yl)quinoline, (15.051) 3-(4,4-difluoro-3,3-dimethyl-3,4-dihydroisoquinoline-1-yl)-8-fluoroquinoline, (15.052) 3-(4,4-difluoro-5,5-dimethyl-4,5-dihydrothieno[2,3-c]pyridin-7-yl)quinoline, (15.053) 3-(5-fluoro-3,3,4,4-tetramethyl-3,4-dihydroisoquinoline-1-yl)quinoline, (15.054) 5-bromo-1-(5,6-dimethylpyridin-3-yl)-3,3-dimethyl-3,4-dihydroisoquinoline, (15.055) 8-fluoro-3-(5-fluoro-3,3,4,4-tetramethyl-3,4-dihydroisoquinoline-1-yl)quinoline, (15.056) 8-fluoro-3-(5-fluoro-3,3-dimethyl-3,4-dihydroisoquinoline-1-yl)quinoline, (15.057) 8-fluoro-N-(4,4,4-trifluoro-2-methyl-1-phenylbutan-2-yl)quinoline-3-carboxamide, (15.058) 8-fluoro-N-[(2S)-4,4,4-trifluoro-2-methyl-1-phenylbutan-2-yl]quinoline-3-carboxamide, (15.059) 9-fluoro-2,2-dimethyl-5-(quinolin-3-yl)-2,3-dihydro-1,4-benzoxazepine, (15.060)N-(2,4-dimethyl-1-phenylpentan-2-yl)-8-fluoroquinoline-3-carboxamide, (15.061)N-[(2S)-2,4-dimethyl-1-phenylpentan-2-yl]-8-fluoroquinoline-3-carboxamide, (15.062) 1,1-diethyl-3-[[4-[5-(trifluoromethyl)-1,2,4-oxadiazol-3-yl]phenyl]methyl]urea, (15.063) 1,3-dimethoxy-1-[[4-[5-(trifluoromethyl)-1,2,4-oxadiazol-3-yl]phenyl]methyl]urea, (15.064) 1-[[3-fluoro-4-(5-(trifluoromethyl)-1,2,4-oxadiazol-3-yl)phenyl]methyl]azepan-2-one, (15.065) 1-[[4-[5-(trifluoromethyl)-1,2,4-oxadiazol-3-yl]phenyl]methyl]piperidin-2-one, (15.066) 1-methoxy-1-methyl-3-[[4-[5-(trifluoromethyl)-1,2,4-oxadiazol-3-yl]phenyl]methyl]urea, (15.067) 1-methoxy-3-methyl-1-[[4-[5-(trifluoromethyl)-1,2,4-oxadiazol-3-yl]phenyl]methyl]urea, (15.068) 1-methoxy-3-methyl-1-[[4-[5-(trifluoromethyl)-1,2,4-oxadiazol-3-yl]phenyl]methyl]urea, (15.069) 2,2-difluoro-N-methyl-2-[4-[5-(trifluoromethyl)-1,2,4-oxadiazol-3-yl]phenyl]acetamide, (15.070) 3,3-dimethyl-1-[[4-[5-(trifluoromethyl)-1,2,4-oxadiazol-3-yl]phenyl]methyl]piperidin-2-one, (15.071) 3-ethyl-1-methoxy-1-[[4-[5-(trifluoromethyl)-1,2,4-oxadiazol-3-yl]phenyl]methyl]urea, (15.072) 4,4-dimethyl-1-[[4-[5-(trifluoromethyl)-1,2,4-oxadiazol-3-yl]phenyl]methyl]pyrrolidin-2-one, (15.073) 4,4-dimethyl-2-[[4-[5-(trifluoromethyl)-1,2,4-oxadiazol-3-yl]phenyl]methyl]isoxazolidin-3-one, (15.074) 4-[5-(trifluoromethyl)-1,2,4-oxadiazol-3-yl]phenyl dimethylcarbamate, (15.075) 5,5-dimethyl-2-[[4-[5-(trifluoromethyl)-1,2,4-oxadiazol-3-yl]phenyl]methyl]isoxazolidin-3-one, (15.076) 5-methyl-1-[[4-[5-(trifluoromethyl)-1,2,4-oxadiazol-3-yl]phenyl]methyl]pyrrolidin-2-one, (15.077) ethyl 1-{4-[5-(trifluoromethyl)-1,2,4-oxadiazol-3-yl]benzyl}-1H-pyrazole-4-carboxylate, (15.078) methyl {4-[5-(trifluoromethyl)-1,2,4-oxadiazol-3-yl]phenyl}carbamate, (15.079)N-(1-methylcyclopropyl)-4-[5-(trifluoromethyl)-1,2,4-oxadiazol-3-yl]benzamide, (15.080)N-(2,4-difluorophenyl)-4-[5-(trifluoromethyl)-1,2,4-oxadiazol-3-yl]benzamide, (15.081)N-(2-fluorophenyl)-4-[5-(trifluoromethyl)-1,2,4-oxadiazol-3-yl]benzamide, (15.082) N,2-dimethoxy-N-[[4-[5-(trifluoromethyl)-1,2,4-oxadiazol-3-yl]phenyl]methyl]propanamide, (15.083) N,N-dimethyl-1-{4-[5-(trifluoromethyl)-1,2,4-oxadiazol-3-yl]benzyl}-1H-1,2,4-triazole-3-amine, (15.084)N-[(E)-methoxyiminomethyl]-4-[5-(trifluoromethyl)-1,2,4-oxadiazol-3-yl]benzamide, (15.085)N-[(E)-N-methoxy-C-methylcarbonimidoyl]-4-[5-(trifluoromethyl)-1,2,4-oxadiazol-3-yl]benzamide, (15.086)N-[(Z)-methoxyiminomethyl]-4-[5-(trifluoromethyl)-1,2,4-oxadiazol-3-yl]benzamide, (15.087)N-[(Z)-N-methoxy-C-methylcarbonimidoyl]-4-[5-(trifluoromethyl)-1,2,4-oxadiazol-3-yl]benzamide, (15.088)N-[[2,3-difluoro-4-[5-(trifluoromethyl)-1,2,4-oxadiazol-3-yl]phenyl]methyl]-3,3,3-trifluoropropanamide, (15.089)N-[[4-[5-(trifluoromethyl)-1,2,4-oxadiazol-3-yl]phenyl]methyl]propanamide, (15.090)N-[4-[5-trifluoromethyl)-1,2,4-oxadiazol-3-yl]phenyl]cyclopropanecarboxamide, (15.091) N-{2,3-difluoro-4-[5-(trifluoromethyl)-1,2,4-oxadiazol-3-yl]benzyl}butanamide, (15.092)N-{4-[5-(trifluoromethyl)-1,2,4-oxadiazol-3-yl]benzyl}cyclopropanecarboxamide, (15.093)N-{4-[5-(trifluoromethyl)-1,2,4-oxadiazol-3-yl]phenyl}propanamide, (15.094)N-allyl-N-[[4-[5-(trifluoromethyl)-1,2,4-oxadiazol-3-yl]phenyl]methyl]acetamide, (15.095)N-allyl-N-[[4-[5-(trifluoromethyl)-1,2,4-oxadiazol-3-yl]phenyl]methyl]propanamide, (15.096)N-ethyl-2-methyl-N-[[4-[5-(trifluoromethyl)-1,2,4-oxadiazol-3-yl]phenyl]methyl]propanamide, (15.097)N-methoxy-N-[[4-[5-(trifluoromethyl)-1,2,4-oxadiazol-3-yl]phenyl]methyl]cyclopropanecarboxamide, (15.098)N-methyl-4-[5-(trifluoromethyl)-1,2,4-oxadiazol-3-yl]benzamide, (15.099)N-methyl-4-[5-(trifluoromethyl)-1,2,4-oxadiazol-3-yl]benzencarbothioamide, (15.100)N-methyl-N-phenyl-4-[5-(trifluoromethyl)-1,2,4-oxadiazol-3-yl]benzamide.

### Biological Pesticides as Mixture Components

The compounds of the formula (I) can be combined with biological pesticides.

Biological pesticides especially include bacteria, fungi, yeasts, plant extracts and such products formed by microorganisms, including proteins and secondary metabolites.

Biological pesticides include bacteria such as spore-forming bacteria, root-colonizing bacteria and bacteria which act as biological insecticides, fungicides or nematicides.

Examples of such bacteria which are used or can be used as biological pesticides are:

Bacillus amyloliquefaciens, strain FZB42 (DSM 231179), or Bacillus cereus, in particular B. cereus strain CNCM I-1562 or Bacillus firmus, strain I-1582 (Accession number CNCM I-1582) or Bacillus pumilus, in particular strain GB34 (Accession No. ATCC 700814) and strain QST2808 (Accession No. NRRL B-30087), or Bacillus subtilis, in particular strain GB03 (Accession No. ATCC SD-1397), or Bacillus subtilis strain QST713 (Accession No. NRRL B-21661) or Bacillus subtilis strain OST 30002 (Accession No. NRRL B-50421), Bacillus thuringiensis, in particular B. thuringiensis subspecies israelensis (Serotype H-14), strain AM65-52 (Accession No. ATCC 1276), or B. thuringiensis subsp. aizawai, in particular strain ABTS-1857 (SD-1372), or B. thuringiensis subsp. kurstaki strain HD-1, or B. thuringiensis subsp. tenebrionis strain NB 176 (SD-5428), Pasteuria penetrans, Pasteuria spp. (Rotylenchulus reniformis nematode)-PR3 (Accession Number ATCC SD-5834), Streptomyces microflavus strain AQ6121 (=QRD 31.013, NRRL B-50550), Streptomyces galbus strain AQ 6047 (Accession Number NRRL 30232).

Examples of fungi and yeasts which are used or can be used as biological pesticides are:

Beauveria bassiana, especially strain ATCC 74040, Coniothyrium minitans, especially strain CON/M/91-8 (Accession No. DSM-9660), Lecanicillium spp., especially strain HRO LEC 12, Lecanicillium lecanii (formerly known as Verticillium lecanii), especially strain KV01, Metarhizium anisopliae, especially strain F52 (DSM3884/ATCC 90448), Metschnikowia fructicola, especially strain NRRL Y-30752, Paecilomyces fumosoroseus (new: Isaria fumosorosea), especially strain IFPC 200613, or strain Apopka 97 (Accession No. ATCC 20874), Paecilomyces lilacinus, especially P. lilacinus strain 251 (AGAL 89/030550), Talaromyces flavus, especially strain V117b, Trichoderma atroviride, especially strain SC1 (Accession Number CBS 122089), Trichoderma harzianum, especially T. harzianum rifai T39 (Accession Number CNCM I-952).

Examples of viruses which are used or can be used as biological pesticides are:

Adoxophyes orana (summer fruit tortrix) granulosis virus (GV), Cydia pomonella (codling moth) granulosis virus (GV), Helicoverpa armigera (cotton bollworm) nuclear polyhedrosis virus (NPV), Spodoptera exigua (beet armyworm) mNPV, Spodoptera frugiperda (fall armyworm) mNPV, Spodoptera littoralis (African cotton leafworm) NPV.

Also included are bacteria and fungi which are added as ‘inoculant’ to plants or plant parts or plant organs and which, by virtue of their particular properties, promote plant growth and plant health. Examples include:

Agrobacterium spp., Azorhizobium caulinodans, Azospirillum spp., Azotobacter spp., Bradyrhizobium spp., Burkholderia spp., especially Burkholderia cepacia (formerly known as Pseudomonas cepacia), Gigaspora spp., or Gigaspora monosporum, Glomus spp., Laccaria spp., Lactobacillus buchneri, Paraglomus spp., Pisolithus tinctorus, Pseudomonas spp., Rhizobium spp., especially Rhizobium trifolii, Rhizopogon spp., Scleroderma spp., Suillus spp., Streptomyces spp.

Examples of plant extracts and products formed by microorganisms, including proteins and secondary metabolites, which are used or can be used as biological pesticides are: Allium sativum, Artemisia absinthium, azadirachtin, Biokeeper WP, Cassia nigricans, Celastrus angulatus, Chenopodium anthelminticum, chitin, Armour-Zen, Dryopteris filix-mas, Equisetum arvense, Fortune Aza, Fungastop, Heads Up (Chenopodium quinoa saponin extract), pyrethrum/pyrethrins, Quassia amara, Quercus, Quillaja, Regalia, “Requiem™ Insecticide”, rotenone, ryania/ryanodine, Symphytum officinale, Tanacetum vulgare, thymol, Triact 70, TriCon, Tropaeulum majus, Urtica dioica, Veratrin, Viscum album, Brassicaceae extract, especially oilseed rape powder or mustard powder, and also active bioinsecticidal/acaricidal ingredients obtained from olive oil, especially unsaturated fatty/carboxylic acids having C₁₆-C₂₀ carbon chain lengths as active ingredients, as contained, for example, in the product with the trade name FLiPPER®.

### Safeners as Mixture Components

The compounds of formula (I) can be combined with safeners, for example benoxacor, cloquintocet (-mexyl), cyometrinil, cyprosulfamide, dichlormid, fenchlorazole (-ethyl), fenclorim, flurazole, fluxofenim, furilazole, isoxadifen (-ethyl), mefenpyr (-diethyl), naphthalic anhydride, oxabetrinil, 2-methoxy-N-({4-[(methylcarbamoyl)amino]phenyl}sulfonyl)benzamide (CAS 129531-12-0), 4-(dichloroacetyl)-1-oxa-4-azaspiro[4.5]decane (CAS 71526-07-3), 2,2,5-trimethyl-3-(dichloroacetyl)-1,3-oxazolidine (CAS 52836-31-4).

### Plants and Plant Parts

All plants and plant parts can be treated in accordance with the invention. Plants are understood here to mean all plants and parts of plants, such as desirable and undesirable wild plants or crop plants (including naturally occurring crop plants), for example cereals (wheat, rice, triticale, barley, rye, oats), maize, soya beans, potatoes, sugar beet, sugar cane, tomatoes, bell peppers, cucumbers, melons, carrots, water melons, onions, lettuce, spinach, leeks, beans, Brassica oleracea (e.g. cabbage) and other vegetable species, cotton, tobacco, oilseed rape, and also fruit plants (the fruits being apples, pears, citrus fruits and grapes). Crop plants may be plants which can be obtained by conventional breeding and optimization methods or by biotechnological and genetic engineering methods or combinations of these methods, including the transgenic plants and including the plant cultivars which are protectable or non-protectable by plant breeders' rights. Plants shall be understood to mean all development stages such as seed, seedlings, young (immature) plants, up to and including mature plants. Plant parts shall be understood to mean all parts and organs of the plants above and below ground, such as shoot, leaf, flower and root, examples given being leaves, needles, stalks, stems, flowers, fruit bodies, fruits and seeds, and also roots, tubers and rhizomes. Plant parts also include harvested plants or harvested plant parts and vegetative and generative propagation material, for example cuttings, tubers, rhizomes, slips and seeds.

The inventive treatment of the plants and parts of plants with the compounds of the formula (I) is effected directly or by allowing the compounds to act on the surroundings, the habitat or the storage space thereof by the customary treatment methods, for example by dipping, spraying, evaporating, fogging, scattering, painting on, injecting, and, in the case of propagation material, especially in the case of seeds, also by applying one or more coats.

As already mentioned above, it is possible to treat all plants and their parts in accordance with the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (genetically modified organisms), and parts thereof, are treated. The term “parts” or “parts of plants” or “plant parts” has been explained above. Particular preference is given in accordance with the invention to treating plants of the respective commercially customary plant cultivars or those that are in use. Plant cultivars are understood to mean plants having new properties (“traits”) and which have been obtained by conventional breeding, by mutagenesis or by recombinant DNA techniques. They may be cultivars, varieties, biotypes and genotypes.

### Transgenic Plants, Seed Treatment and Integration Events

According to the invention, the compounds of formula (I) can be employed advantageously for treating transgenic plants, plant cultivars or plant parts which have received genetic material which confers advantageous and/or useful properties (traits) to these plants, plant cultivars and plant parts, respectively. One option is therefore to combine the present invention with one or more recombinant traits or transgenic events or a combination thereof. For the purposes of the present application, a transgenic event is caused by insertion of a specific recombinant DNA molecule into a specific position (locus) in the chromosome of the plant genome. The insertion creates a new DNA sequence called an “event”, which is characterized by the inserted recombinant DNA molecule and a certain amount of genomic DNA directly adjacent to the inserted DNA/flanking the inserted DNA on both ends. Such traits or transgenic events include, without limitation, pest resistance, water utilization efficiency, yield performance, drought tolerance, seed quality, improved nutrient quality, hybrid seed production and herbicide tolerance, the trait being measured in comparison to a plant lacking such a trait or such a transgenic event. Specific examples of such advantageous and/or useful properties (traits) are better plant growth, vigour, stress tolerance, stability, resistance to lodging, nutrient uptake, plant nutrition and/or yield, in particular improved growth, increased tolerance to high or low temperatures, increased tolerance to drought or to water or soil salt content, increased flowering performance, easier harvesting, accelerated maturation, higher yields, better quality and/or a higher nutritional value of the harvested products, better storage stability and/or processibility of the harvested products and increased resistance or tolerance to animal or microbial pests such as against insects, arachnids, nematodes, mites and slugs and snails.

From the DNA sequences coding for proteins conferring traits of resistance or tolerance to such animal or microbial pests, in particular insects, particular mention may be made of the genetic material of Bacillus thuringiensis coding for Bt proteins, which are described in detail in the literature and are familiar to the person skilled in the art. Mention should also be made of proteins extracted from bacteria such as Photorhabdus (WO97/17432 and WO98/08932). Particular mention may be made of Bt-Cry or VIP proteins, which include the Cry1A, CryIAb, CryIAc, CryIIA, CryIIIA, CryIIIB2, Cry9c, Cry2Ab, Cry3Bb and CryIF proteins or toxic fragments thereof, and furthermore hybrids or combinations thereof, in particular the CryiF protein or hybrids derived from a Cry1F protein (e.g. hybrid Cry1A-Cry1F proteins or toxic fragments thereof), the proteins of the Cry1A type or toxic fragments thereof, preferably the Cry1Ac protein or hybrids derived from the Cry1Ac protein (e.g. hybrid Cry1Ab-Cry1Ac proteins) or the Cry1Ab or Bt2 protein or toxic fragments thereof, the Cry2Ae, Cry2Af or Cry2Ag proteins or toxic fragments thereof, the Cry1A.105 protein or a toxic fragment thereof, the VIP3Aa19 protein, the VIP3Aa20 protein, the VIP3A proteins, which are produced in the COT202 or COT203 cotton events, the VIP3Aa protein or a toxic fragment thereof, as described in Estruch et al. (1996), Proc Natl Acad Sci USA. 28; 93(11):5389-94, the Cry proteins, as described in WO2001/47952, the insecticidal proteins from Xenorhabdus (as described in WO98/50427), Serratia (in particular from S. entomophila) or strains of the Photorhabdus species, such as Tc proteins from Photorhabdus, as described in WO98/08932. This also includes all variants and mutants of any of these proteins which differ in a number of amino acids (1-10, preferably 1-5) from any of the sequences listed above, in particular from the sequence of its toxic fragment, or which are fused with a transit peptide such as a plastid transit peptide or another protein or peptide.

Another and particularly emphasized example of such properties is a conferred tolerance to one or more herbicides, for example imidazolinones, sulfonylureas, glyphosate or phosphinothricin. Of the DNA sequences coding for proteins which confer traits of tolerance to certain herbicides to the transformed plant cells and plants, particular mention may be made of the bar or PAT gene or the Streptomyces coelicolor gene described in WO2009/152359, which confers tolerance to glufosinate herbicides, a gene which codes for a suitable EPSPS (5-enolpyruvylshikimate 3-phosphate synthase), which confers tolerance to herbicides with EPSPS as target, in particular herbicides such as glyphosate and its salts, a gene coding for glyphosate N-acetyltransferase or a gene coding for glyphosate oxoreductase. Further suitable herbicide tolerance traits include at least one ALS (acetolactate synthase) inhibitor (e.g. WO2007/024782), a mutated Arabidopsis ALS/AHAS gene (e.g. U.S. Pat. No. 6,855,533), genes coding for 2,4-D-monooxygenases, which confer tolerance to 2,4-D (2,4-dichlorophenoxyacetic acid), and genes coding for dicamba monooxygenases which confer tolerance to dicamba (3,6-dichloro-2-methoxybenzoic acid).

Further and particularly emphasized examples of such properties are increased resistance to phytopathogenic fungi, bacteria and/or viruses caused, for example, by systemic acquired resistance (SAR), systemin, phytoalexins, elicitors and also resistance genes and correspondingly expressed proteins and toxins.

Particularly useful transgenic events in transgenic plants or plant cultivars which can be treated with preference in accordance with the invention include event 531/PV-GHBK04 (cotton, insect control, described in WO2002/040677), event 1143-14A (cotton, insect control, not deposited, described in WO2006/128569); event 1143-51B (cotton, insect control, not deposited, described in WO2006/128570); event 1445 (cotton, herbicide tolerance, not deposited, described in US-A 2002-120964 or WO2002/034946); event 17053 (rice, herbicide tolerance, deposited as PTA-9843, described in WO2010/117737); event 17314 (rice, herbicide tolerance, deposited as PTA-9844, described in WO2010/117735); event 281-24-236 (cotton, insect control —herbicide tolerance, deposited as PTA-6233, described in WO2005/103266 or US-A 2005-216969); event 3006-210-23 (cotton, insect control—herbicide tolerance, deposited as PTA-6233, described in US-A 2007-143876 or WO2005/103266); event 3272 (maize, quality trait, deposited as PTA-9972, described in WO2006/098952 or US-A 2006-230473); event 33391 (wheat, herbicide tolerance, deposited as PTA-2347, described in WO2002/027004), event 40416 (maize, insect control—herbicide tolerance, deposited as ATCC PTA-11508, described in WO 11/075593); event 43A47 (maize, insect control—herbicide tolerance, deposited as ATCC PTA-11509, described in WO2011/075595); event 5307 (maize, insect control, deposited as ATCC PTA-9561, described in WO2010/077816); event ASR-368 (bentgrass, herbicide tolerance, deposited as ATCC PTA-4816, described in US-A 2006-162007 or WO2004/053062); event B16 (maize, herbicide tolerance, not deposited, described in US-A 2003-126634); event BPS-CV127-9 (soya bean, herbicide tolerance, deposited as NCIMB no. 41603, described in WO2010/080829); event BLR1 (oilseed rape, restauration of male sterility, deposited as NCIMB 41193, described in WO2005/074671), event CE43-67B (cotton, insect control, deposited as DSM ACC2724, described in US-A 2009-217423 or WO2006/128573); event CE44-69D (cotton, insect control, not deposited, described in US-A 2010-0024077); event CE44-69D (cotton, insect control, not deposited, described in WO2006/128571); event CE46-02A (cotton, insect control, not deposited, described in WO2006/128572); event COT102 (cotton, insect control, not deposited, described in US-A 2006-130175 or WO2004/039986); event COT202 (cotton, insect control, not deposited, described in US-A 2007-067868 or WO2005/054479); event COT203 (cotton, insect control, not deposited, described in WO2005/054480); event DAS21606-3/1606 (soya bean, herbicide tolerance, deposited as PTA-11028, described in WO2012/033794), event DAS40278 (maize, herbicide tolerance, deposited as ATCC PTA-10244, described in WO2011/022469); event DAS-44406-6/pDAB8264.44.06.1 (soya bean, herbicide tolerance, deposited as PTA-11336, described in WO2012/075426), event DAS-14536-7/pDAB8291.45.36.2 (soya bean, herbicide tolerance, deposited as PTA-11335, described in WO2012/075429), event DAS-59122-7 (maize, insect control—herbicide tolerance, deposited as ATCC PTA 11384, described in US-A 2006-070139); event DAS-59132 (maize, insect control—herbicide tolerance, not deposited, described in WO2009/100188); event DAS68416 (soya bean, herbicide tolerance, deposited as ATCC PTA-10442, described in WO2011/066384 or WO2011/066360); event DP-098140-6 (maize, herbicide tolerance, deposited as ATCC PTA-8296, described in US-A 2009-137395 or WO 08/112019); event DP-305423-1 (soya bean, quality trait, not deposited, described in US-A 2008-312082 or WO2008/054747); event DP-32138-1 (maize, hybridization system, deposited as ATCC PTA-9158, described in US-A 2009-0210970 or WO2009/103049); event DP-356043-5 (soya bean, herbicide tolerance, deposited as ATCC PTA-8287, described in US-A 2010-0184079 or WO2008/002872); event EE-I (aubergine, insect control, not deposited, described in WO 07/091277); event Fil 17 (maize, herbicide tolerance, deposited as ATCC 209031, described in US-A 2006-059581 or WO 98/044140); event FG72 (soya bean, herbicide tolerance, deposited as PTA-11041, described in WO2011/063413), event GA21 (maize, herbicide tolerance, deposited as ATCC 209033, described in US-A 2005-086719 or WO 98/044140); event GG25 (maize, herbicide tolerance, deposited as ATCC 209032, described in US-A 2005-188434 or WO98/044140); event GHB119 (cotton, insect control—herbicide tolerance, deposited as ATCC PTA-8398, described in WO2008/151780); event GHB614 (cotton, herbicide tolerance, deposited as ATCC PTA-6878, described in US-A 2010-050282 or WO2007/017186); event GJ11 (maize, herbicide tolerance, deposited as ATCC 209030, described in US-A 2005-188434 or WO98/044140); event GM RZ13 (sugar beet, virus resistance, deposited as NCIMB-41601, described in WO2010/076212); event H7-1 (sugar beet, herbicide tolerance, deposited as NCIMB 41158 or NCIMB 41159, described in US-A 2004-172669 or WO 2004/074492); event JOPLIN1 (wheat, disease tolerance, not deposited, described in US-A 2008-064032); event LL27 (soya bean, herbicide tolerance, deposited as NCIMB41658, described in WO2006/108674 or US-A 2008-320616); event LL55 (soya bean, herbicide tolerance, deposited as NCIMB 41660, described in WO 2006/108675 or US-A 2008-196127); event LLcotton25 (cotton, herbicide tolerance, deposited as ATCC PTA-3343, described in WO2003/013224 or US-A 2003-097687); event LLRICE06 (rice, herbicide tolerance, deposited as ATCC 203353, described in U.S. Pat. No. 6,468,747 or WO2000/026345); event LLRice62 (rice, herbicide tolerance, deposited as ATCC 203352, described in WO2000/026345), event LLRICE601 (rice, herbicide tolerance, deposited as ATCC PTA-2600, described in US-A 2008-2289060 or WO2000/026356); event LY038 (maize, quality trait, deposited as ATCC PTA-5623, described in US-A 2007-028322 or WO2005/061720); event MIR162 (maize, insect control, deposited as PTA-8166, described in US-A 2009-300784 or WO2007/142840); event MIR604 (maize, insect control, not deposited, described in US-A 2008-167456 or WO2005/103301); event MON15985 (cotton, insect control, deposited as ATCC PTA-2516, described in US-A 2004-250317 or WO2002/100163); event MON810 (maize, insect control, not deposited, described in US-A 2002-102582); event MON863 (maize, insect control, deposited as ATCC PTA-2605, described in WO2004/011601 or US-A 2006-095986); event MON87427 (maize, pollination control, deposited as ATCC PTA-7899, described in WO2011/062904); event MON87460 (maize, stress tolerance, deposited as ATCC PTA-8910, described in WO2009/111263 or US-A 2011-0138504); event MON87701 (soya bean, insect control, deposited as ATCC PTA-8194, described in US-A 2009-130071 or WO2009/064652); event MON87705 (soya bean, quality trait—herbicide tolerance, deposited as ATCC PTA-9241, described in US-A 2010-0080887 or WO2010/037016); event MON87708 (soya bean, herbicide tolerance, deposited as ATCC PTA-9670, described in WO2011/034704); event MON87712 (soya bean, yield, deposited as PTA-10296, described in WO2012/051199), event MON87754 (soya bean, quality trait, deposited as ATCC PTA-9385, described in WO2010/024976); event MON87769 (soya bean, quality trait, deposited as ATCC PTA-8911, described in US-A 2011-0067141 or WO2009/102873); event MON88017 (maize, insect control—herbicide tolerance, deposited as ATCC PTA-5582, described in US-A 2008-028482 or WO2005/059103); event MON88913 (cotton, herbicide tolerance, deposited as ATCC PTA-4854, described in WO2004/072235 or US-A 2006-059590); event MON88302 (oilseed rape, herbicide tolerance, deposited as PTA-10955, described in WO2011/153186), event MON88701 (cotton, herbicide tolerance, deposited as PTA-11754, described in WO2012/134808), event MON89034 (maize, insect control, deposited as ATCC PTA-7455, described in WO 07/140256 or US-A 2008-260932); event MON89788 (soya bean, herbicide tolerance, deposited as ATCC PTA-6708, described in US-A 2006-282915 or WO2006/130436); event MS1 1 (oilseed rape, pollination control—herbicide tolerance, deposited as ATCC PTA-850 or PTA-2485, described in WO2001/031042); event MS8 (oilseed rape, pollination control—herbicide tolerance, deposited as ATCC PTA-730, described in WO2001/041558 or US-A 2003-188347); event NK603 (maize, herbicide tolerance, deposited as ATCC PTA-2478, described in US-A 2007-292854); event PE-7 (rice, insect control, not deposited, described in WO2008/114282); event RF3 (oilseed rape, pollination control—herbicide tolerance, deposited as ATCC PTA-730, described in WO2001/041558 or US-A 2003-188347); event RT73 (oilseed rape, herbicide tolerance, not deposited, described in WO2002/036831 or US-A 2008-070260); event SYHT0H2/SYN-000H2-5 (soya bean, herbicide tolerance, deposited as PTA-11226, described in WO2012/082548), event T227-1 (sugar beet, herbicide tolerance, not deposited, described in WO2002/44407 or US-A 2009-265817); event T25 (maize, herbicide tolerance, not deposited, described in US-A 2001-029014 or WO2001/051654); event T304-40 (cotton, insect control—herbicide tolerance, deposited as ATCC PTA-8171, described in US-A 2010-077501 or WO2008/122406); event T342-142 (cotton, insect control, not deposited, described in WO2006/128568); event TC1507 (maize, insect control—herbicide tolerance, not deposited, described in US-A 2005-039226 or WO2004/099447); event VIP1034 (maize, insect control —herbicide tolerance, deposited as ATCC PTA-3925, described in WO2003/052073), event 32316 (maize, insect control—herbicide tolerance, deposited as PTA-11507, described in WO2011/084632), event 4114 (maize, insect control—herbicide tolerance, deposited as PTA-11506, described in WO2011/084621), event EE-GM3/FG72 (soya bean, herbicide tolerance, ATCC accession no. PTA-11041) optionally stacked with event EE-GM1/LL27 or event EE-GM2/LL55 (WO2011/063413A2), event DAS-68416-4 (soya bean, herbicide tolerance, ATCC accession no. PTA-10442, WO2011/066360A1), event DAS-68416-4 (soya bean, herbicide tolerance, ATCC accession no. PTA-10442, WO2011/066384A1), event DP-040416-8 (maize, insect control, ATCC accession no. PTA-11508, WO2011/075593A1), event DP-043A47-3 (maize, insect control, ATCC accession no. PTA-11509, WO2011/075595A1), event DP-004114-3 (maize, insect control, ATCC accession no. PTA-11506, WO2011/084621A1), event DP-032316-8 (maize, insect control, ATCC accession no. PTA-11507, WO2011/084632A1), event MON-88302-9 (oilseed rape, herbicide tolerance, ATCC accession no. PTA-10955, WO2011/153186A1), event DAS-21606-3 (soya bean, herbicide tolerance, ATCC accession no. PTA-11028, WO2012/033794A2), event MON-87712-4 (soya bean, quality trait, ATCC accession no. PTA-10296, WO2012/051199A2), event DAS-44406-6 (soya bean, stacked herbicide tolerance, ATCC accession no. PTA-11336, WO2012/075426A1), event DAS-14536-7 (soya bean, stacked herbicide tolerance, ATCC accession no. PTA-11335, WO2012/075429A1), event SYN-000H2-5 (soya bean, herbicide tolerance, ATCC accession no. PTA-11226, WO2012/082548A2), event DP-061061-7 (oilseed rape, herbicide tolerance, no deposition no. available, WO2012071039A1), event DP-073496-4 (oilseed rape, herbicide tolerance, no deposition no. available, US2012131692), event 8264.44.06.1 (soya bean, stacked herbicide tolerance, accession no. PTA-11336, WO2012075426A2), event 8291.45.36.2 (soya bean, stacked herbicide tolerance, accession no. PTA-11335, WO2012075429A2), event SYHTOH2 (soya bean, ATCC accession no. PTA-11226, WO2012/082548A2), event MON88701 (cotton, ATCC accession no. PTA-11754, WO2012/134808A1), event KK179-2 (alfalfa, ATCC accession no. PTA-11833, WO2013/003558A1), event pDAB8264.42.32.1 (soya bean, stacked herbicide tolerance, ATCC accession no. PTA-11993, WO2013/010094A1), event MZDT09Y (maize, ATCC accession no. PTA-13025, WO2013/012775A1).

Furthermore, such a list of transgenic events is provided by the United States Department of Agriculture's (USDA) Animal and Plant Health Inspection Service (APHIS) and is found on their website on the World Wide Web at aphis.usda.gov. For the present application, the status of this list as of the filing date of the present application is of relevance.

In the transgenic plants, the genes/events which confer the desired traits in question can also be present in combination with one another. Examples of transgenic plants which may be mentioned include the important crop plants, such as cereals (wheat, rice, triticale, barley, rye, oats), maize, soya beans, potatoes, sugar beet, sugar cane, tomatoes, peas and other vegetable species, cotton, tobacco, oilseed rape, and also fruit plants (the fruits being apples, pears, citrus fruits and grapes), particular emphasis being given to maize, soya beans, wheat, rice, potatoes, cotton, sugar cane, tobacco and oilseed rape. Traits that should be given particular emphasis are increased resistance of the plants to insects, arachnids, nematodes and slugs and snails, and increased resistance of the plants to one or more herbicides.

Commercially available examples of such plants, plant parts or plant seeds which can preferably be treated according to the invention include commercially available products such as plant seeds sold or available under the GENUITY®, DROUGHTGARD®, SMARTSTAX®, RIB COMPLETE®, ROUNDUP READY®, VT DOUBLE PRO®, VT TRIPLE PRO®, BOLLGARD II®, ROUNDUP READY 2 YIELD®, YIELDGARD®, ROUNDUP READY® 2 XTEN^{DTM}, INTACTA RR2 PRO®, VISTIVE GOLD® and/or XTENDFLEX™ trade names.

### Crop Protection—Types of Treatment

The plants and plant parts are treated with the compounds of the formula (I) directly or by action on their surroundings, habitat or storage space using customary treatment methods, for example by dipping, spraying, atomizing, irrigating, evaporating, dusting, fogging, broadcasting, foaming, painting, spreading-on, injecting, watering (drenching), drip irrigating and, in the case of propagation material, in particular in the case of seed, additionally by dry seed treatment, liquid seed treatment, slurry treatment, by incrusting, by coating with one or more coats, etc. It is furthermore possible to apply the compounds of the formula (I) by the ultra-low volume method or to inject the application form or the compound of the formula (I) itself into the soil.

A preferred direct treatment of the plants is foliar application, meaning that the compounds of the formula (I) are applied to the foliage, in which case the treatment frequency and the application rate should be adjusted according to the level of infestation with the pest in question.

In the case of systemically active ingredients, the compounds of the formula (I) also access the plants via the root system. The plants are then treated by the action of the compounds of the formula (I) on the habitat of the plant. This can be accomplished, for example, by drenching, or by mixing into the soil or the nutrient solution, meaning that the locus of the plant (e.g. soil or hydroponic systems) is impregnated with a liquid form of the compounds of the formula (I), or by soil application, meaning that the compounds of the formula (I) of the invention are introduced in solid form (e.g. in the form of granules) into the locus of the plants, or by drip application (frequently also referred to as “chemigation”), meaning that the compounds of the formula (I) of the invention are introduced via surface or underground drip lines over certain periods of time together with varying amounts of water at defined locations in the vicinity of the plants. In the case of paddy rice crops, this can also be accomplished by metering the compound of the formula (I) in a solid application form (for example as granules) into a flooded paddy field.

### Digital Technologies

The compounds of the invention can be used in combination with, for example, models embedded in computer programs for site-specific crop plant management, satellite agriculture, precision agriculture or precision farming. Such models support the site-specific management of agricultural facilities with data from different sources such as soil, weather, crop plants (e.g. type, growth stage, plant health), weeds (e.g. type, growth stage), diseases, pests, nutrients, water, humidity, biomass, satellite data, yield etc., with the aim to optimize profitability, sustainability and environmental protection. Such models may help in particular to optimize agronomical decisions, to control the precision of pesticide applications and to monitor the operations carried out.

For example, the compounds of the invention can be applied to a crop plant according to an appropriate use protocol if the model modulates the occurrence of a pest and calculates that a threshold has been reached where it is recommended to apply the compound of the invention to the crop plant.

Commercially available systems including agronomic models are, for example, FieldScripts™ from The Climate Corporation, Xarvio™ from BASF, AGLogic™ from John Deere etc.

Moreover, the compounds of the invention can be used in combination with smart sprayers such as equipment for selective spraying or precision spraying attached to or integrated in a farm vehicle such as a tractor, a robot, a helicopter, a plane, an unmanned aerial vehicle (UAV) such as a drone. Such equipment usually comprises input sensors (for example a camera) and a processing unit configured for the analysis of the input data and the provision of a decision based on the analysis of the input data, for the specific and precise application of the compound of the invention to the crop plants (or weeds). The use of such smart sprayers usually requires positioning systems (for example GPS receivers) which localize the acquired data and steer or control farm vehicles, geographic information systems (GIS) which represent the information on comprehensible maps and corresponding farm vehicles for carrying out the required agricultural action such as spraying.

In one example, pests can be detected from pictures taken by a camera. In one example, the pests can be identified and/or classified based on these pictures. In such an identification and/or classification, image processing algorithms may be employed. Such algorithms for image processing may be algorithms for machine learning such as artificial neural networks, decision trees and artificial intelligence algorithms. In this manner, it is possible to apply the compounds described herein only where they are needed.

### Seed Treatment

The control of animal pests by the treatment of the seed of plants has long been known and is the subject of constant improvements. Nevertheless, the treatment of seed entails a series of problems which cannot always be solved in a satisfactory manner. Thus, it is desirable to develop methods for protecting the seed and the germinating plant which dispense with, or at least reduce considerably, the additional application of pesticides during storage, after sowing or after emergence of the plants. It is additionally desirable to optimize the amount of active ingredient used so as to provide optimum protection for the seed and the germinating plant from attack by animal pests, but without damage to the plant itself by the active ingredient used. In particular, methods for the treatment of seed should also take account of the intrinsic insecticidal or nematicidal properties of pest-resistant or -tolerant transgenic plants in order to achieve optimal protection of the seed and also the germinating plant with a minimum expenditure on pesticides.

The present invention therefore in particular also relates to a method for the protection of seed and germinating plants from attack by pests, by treating the seed with one of the compounds of the formula (I). The method of the invention for protecting seed and germinating plants against attack by pests further comprises a method in which the seed is treated simultaneously in one operation or sequentially with a compound of the formula (I) and a mixing component. It further also comprises a method where the seed is treated at different times with a compound of the formula (I) and a mixing component.

The invention likewise relates to the use of the compounds of formula (I) for the treatment of seed for protecting the seed and the resulting plant from animal pests.

The invention further relates to seed which has been treated with an inventive compound of the formula (I) for protection from animal pests. The invention also relates to seed which has been treated simultaneously with a compound of the formula (I) and a mixing component. The invention further relates to seed which has been treated at different times with a compound of the formula (I) and a mixing component. In the case of seed which has been treated at different times with a compound of the formula (I) and a mixing component, the individual substances may be present on the seed in different layers. In this case, the layers comprising a compound of the formula (I) and mixing components may optionally be separated by an intermediate layer. The invention also relates to seed in which a compound of the formula (I) and a mixing component have been applied as part of a coating or as a further layer or further layers in addition to a coating.

The invention further relates to seed which, after the treatment with a compound of the formula (I), is subjected to a film-coating process to prevent dust abrasion on the seed.

One of the advantages that occurs when a compound of the formula (I) acts systemically is that the treatment of the seed protects not only the seed itself but also the plants resulting therefrom, after emergence, from animal pests. In this way, the immediate treatment of the crop at the time of sowing or shortly thereafter can be dispensed with.

A further advantage is that the treatment of the seed with a compound of the formula (I) can enhance germination and emergence of the treated seed.

It is likewise considered to be advantageous that compounds of the formula (I) can especially also be used for transgenic seed.

Furthermore, compounds of the formula (I) can be used in combination with compositions or compounds for signalling technology, leading to better colonization by symbionts, for example rhizobia, mycorrhizae and/or endophytic bacteria or fungi, and/or to optimized nitrogen fixation.

The compounds of the formula (I) are suitable for protection of seed of any plant variety which is used in agriculture, in the greenhouse, in forests or in horticulture. More particularly, the seed is that of cereals (for example wheat, barley, rye, millet and oats), maize, cotton, soya beans, rice, potatoes, sunflowers, coffee, tobacco, canola, oilseed rape, beets (for example sugar beets and fodder beets), peanuts, vegetables (for example tomatoes, cucumbers, beans, cruciferous vegetables, onions and lettuce), fruit plants, lawns and ornamental plants. Of particular significance is the treatment of the seed of cereals (such as wheat, barley, rye and oats), maize, soya beans, cotton, canola, oilseed rape, vegetables and rice.

As already mentioned above, the treatment of transgenic seed with a compound of the formula (I) is also of particular importance. This involves the seed of plants which generally contain at least one heterologous gene which controls the expression of a polypeptide having insecticidal and/or nematicidal properties in particular. The heterologous genes in transgenic seed may originate from microorganisms such as Bacillus, Rhizobium, Pseudomonas, Serratia, Trichoderma, Clavibacter, Glomus or Gliocladium. The present invention is particularly suitable for treatment of transgenic seed which comprises at least one heterologous gene originating from Bacillus sp. The heterologous gene is more preferably derived from Bacillus thuringiensis.

In the context of the present invention, the compound of the formula (I) is applied to the seed. The seed is preferably treated in a state in which it is sufficiently stable for no damage to occur in the course of treatment. In general, the seed can be treated at any time between harvest and sowing. It is customary to use seed which has been separated from the plant and freed from cobs, shells, stalks, coats, hairs or the flesh of the fruits. For example, it is possible to use seed which has been harvested, cleaned and dried down to a moisture content which allows storage. Alternatively, it is also possible to use seed which, after drying, has been treated with, for example, water and then dried again, for example priming. In the case of rice seed, it is also possible to use seed which has been soaked, for example in water, until it reaches a certain stage of the rice embryo (“pigeon breast stage”) which results in stimulation of germination and more uniform emergence.

When treating the seed, care must generally be taken that the amount of the compound of the formula (I) applied to the seed and/or the amount of further additives is chosen in such a way that the germination of the seed is not adversely affected, or that the resulting plant is not damaged. This has to be ensured particularly in the case of active ingredients which can exhibit phytotoxic effects at certain application rates.

In general, the compounds of the formula (I) are applied to the seed in the form of a suitable formulation. Suitable formulations and processes for seed treatment are known to the person skilled in the art.

The compounds of the formula (I) can be converted to the customary seed-dressing formulations, such as solutions, emulsions, suspensions, powders, foams, slurries or other coating compositions for seed, and also ULV formulations.

These formulations are prepared in a known manner, by mixing the compounds of the formula (I) with customary additives, for example customary extenders and solvents or diluents, dyes, wetting agents, dispersants, emulsifiers, antifoams, preservatives, secondary thickeners, adhesives, gibberellins, and also water.

Dyes which may be present in the seed-dressing formulations usable in accordance with the invention are all dyes which are customary for such purposes. It is possible to use either pigments, which are sparingly soluble in water, or dyes, which are soluble in water. Examples include the dyes known by the names Rhodamine B, C.I. Pigment Red 112 and C.I. Solvent Red 1.

Useful wetting agents which may be present in the seed-dressing formulations usable in accordance with the invention are all substances which promote wetting and which are customary for the formulation of agrochemically active ingredients. Usable with preference are alkyl naphthalenesulfonates, such as diisopropyl or diisobutyl naphthalenesulfonates.

Suitable dispersants and/or emulsifiers which may be present in the seed-dressing formulations usable in accordance with the invention are all nonionic, anionic and cationic dispersants customary for the formulation of agrochemically active ingredients. Nonionic or anionic dispersants or mixtures of nonionic or anionic dispersants can be used with preference. Suitable nonionic dispersants especially include ethylene oxide/propylene oxide block polymers, alkylphenol polyglycol ethers and tristyrylphenol polyglycol ethers, and the phosphated or sulfated derivatives thereof. Suitable anionic dispersants are especially lignosulfonates, polyacrylic acid salts and arylsulfonate-formaldehyde condensates.

Antifoams which may be present in the seed-dressing formulations usable in accordance with the invention are all foam-inhibiting substances customary for the formulation of agrochemically active ingredients. Silicone antifoams and magnesium stearate can be used with preference.

Preservatives which may be present in the seed-dressing formulations usable in accordance with the invention are all substances usable for such purposes in agrochemical compositions. Examples include dichlorophene and benzyl alcohol hemiformal.

Secondary thickeners which may be present in the seed-dressing formulations usable in accordance with the invention are all substances which can be used for such purposes in agrochemical compositions. Preferred examples include cellulose derivatives, acrylic acid derivatives, xanthan, modified clays and finely divided silica.

Useful stickers which may be present in the seed-dressing formulations usable in accordance with the invention are all customary binders usable in seed-dressing products. Preferred examples include polyvinylpyrrolidone, polyvinyl acetate, polyvinyl alcohol and tylose.

Gibberellins which may be present in the seed-dressing formulations usable in accordance with the invention are preferably the gibberellins A1, A3 (=gibberellic acid), A4 and A7; particular preference is given to using gibberellic acid. The gibberellins are known (cf. R. Wegler “Chemie der Pflanzenschutz- und Schädlingsbekämpfungsmittel” [Chemistry of Crop Protection Compositions and Pesticides], vol. 2, Springer Verlag, 1970, p. 401-412).

The seed-dressing formulations usable in accordance with the invention can be used to treat a wide variety of different kinds of seed, either directly or after prior dilution with water. For instance, the concentrates or the preparations obtainable therefrom by dilution with water can be used to dress the seed of cereals, such as wheat, barley, rye, oats and triticale, and also the seed of maize, rice, oilseed rape, peas, beans, cotton, sunflowers, soya beans and beets, or else a wide variety of different vegetable seed. The seed-dressing formulations usable in accordance with the invention, or the dilute use forms thereof, can also be used to dress seeds of transgenic plants.

For the treatment of seed with the seed-dressing formulations usable in accordance with the invention, or the use forms prepared therefrom through the addition of water, all mixing units usable customarily for the seed dressing are useful. Specifically, the procedure in seed dressing is to place the seed into a mixer in batchwise or continuous operation, to add the particular desired amount of seed-dressing formulations, either as such or after prior dilution with water, and to mix until the formulation is distributed homogeneously on the seed. If appropriate, this is followed by a drying operation.

The application rate of the seed dressing formulations usable in accordance with the invention can be varied within a relatively wide range. It is guided by the particular content of the compounds of the formula (I) in the formulations and by the seed. The application rates of the compound of the formula (I) are generally between 0.001 and 50 g per kilogram of seed, preferably between 0.01 and 15 g per kilogram of seed.

### Animal Health

In the animal health field, i.e. the field of veterinary medicine, the compounds of the formula (I) are active against animal parasites, in particular ectoparasites or endoparasites. The term “endoparasite” includes especially helminths and protozoa, such as coccidia. Ectoparasites are typically and preferably arthropods, especially insects or acarids.

In the field of veterinary medicine, the compounds of the formula (I) having favourable endotherm toxicity are suitable for controlling parasites which occur in animal breeding and animal husbandry in livestock, breeding animals, zoo animals, laboratory animals, experimental animals and domestic animals. They are active against all or specific stages of development of the parasites.

Agricultural livestock include, for example, mammals, such as sheep, goats, horses, donkeys, camels, buffalo, rabbits, reindeer, fallow deer and especially cattle and pigs; or poultry such as turkeys, ducks, geese and especially chickens; or fish or crustaceans, for example in aquaculture; or, as the case may be, insects such as bees.

Domestic animals include, for example, mammals, such as hamsters, guinea pigs, rats, mice, chinchillas, ferrets, and particularly dogs, cats, caged birds; reptiles, amphibians or aquarium fish.

In a specific embodiment, the compounds of the formula (I) are administered to mammals.

In another specific embodiment, the compounds of the formula (I) are administered to birds, namely caged birds or particularly poultry.

Use of the compounds of the formula (I) for the control of animal parasites is intended to reduce or prevent illness, cases of death and reductions in performance (in the case of meat, milk, wool, hides, eggs, honey and the like), such that more economical and simpler animal husbandry is enabled and better animal well-being is achievable.

In relation to the field of animal health, the term “control” or “controlling” in the present context means that the compounds of the formula (I) are effective in reducing the incidence of the particular parasite in an animal infected with such parasites to an innocuous degree. More specifically, “controlling” in the present context means that the compounds of the formula (I) kill the respective parasite, inhibit its growth, or inhibit its proliferation.

The arthropods include, for example, but are not limited to,
from the order of Anoplurida, for example Haematopinus spp., Linognathus spp., Pediculus spp., Phtirus spp. and Solenopotes spp.;
from the order of Mallophagida and the suborders Amblycerina and Ischnocerina, for example, Bovicola spp., Damalina spp., Felicola spp.; Lepikentron spp., Menopon spp., Trichodectes spp., Trimenopon spp., Trinoton spp., Werneckiella spp;
from the order of Diptera and the suborders Nematocerina and Brachycerina, for example, Aedes spp., Anopheles spp., Atylotus spp., Braula spp., Calliphora spp., Chrysomyia spp., Chrysops spp., Culex spp., Culicoides spp., Eusimulium spp., Fannia spp., Gasterophilus spp., Glossina spp., Haematobia spp., Haematopota spp., Hippobosca spp., Hybomitra spp., Hydrotaea spp., Hypoderma spp., Lipoptena spp., Lucilia spp., Lutzomyia spp., Melophagus spp., Morellia spp., Musca spp., Odagmia spp., Oestrus spp., Philipomyia spp., Phlebotomus spp., Rhinoestrus spp., Sarcophaga spp., Simulium spp., Stomoxys spp., Tabanus spp., Tipula spp., Wilhelmia spp., Wohlfahrtia spp.;
from the order of Siphonapterida, for example, Ceratophyllus spp., Ctenocephalides spp., Pulex spp., Tunga spp., Xenopsylla spp.;
from the order of Heteropterida, for example Cimex spp., Panstrongylus spp., Rhodnius spp., Triatoma spp.; and also nuisance and hygiene pests from the order Blattarida.

In addition, in the case of the arthropods, mention should be made by way of example, without limitation, of the following Acari:
from the subclass of Acari (Acarina) and the order of Metastigmata, for example from the family of Argasidae such as Argas spp., Ornithodorus spp., Otobius spp., from the family of Ixodidae such as Amblyomma spp., Dermacentor spp., Haemaphysalis spp., Hyalomma spp., Ixodes spp., Rhipicephalus (Boophilus) spp., Rhipicephalus spp. (the original genus of multi-host ticks); from the order of Mesostigmata such as Dermanyssus spp., Ornithonyssus spp., Pneumonyssus spp., Raillietia spp., Sternostoma spp., Tropilaelaps spp., Varroa spp.; from the order of the Actinedida (Prostigmata), for example Acarapis spp., Cheyletiella spp., Demodex spp., Listrophorus spp., Myobia spp., Neotrombicula spp., Ornithocheyletia spp., Psorergates spp., Trombicula spp.; and from the order of the Acaridida (Astigmata), for example Acarus spp., Caloglyphus spp., Chorioptes spp., Cytodites spp., Hypodectes spp., Knemidocoptes spp., Laminosioptes spp., Notoedres spp., Otodectes spp., Psoroptes spp., Pterolichus spp., Sarcoptes spp., Trixacarus spp., Tyrophagus spp.

Examples of parasitic protozoa include, but are not limited to:

Mastigophora (Flagellata), such as:
Metamonada: from the order of Diplomonadida, for example Giardia spp., Spironucleus spp.
Parabasala: from the order of Trichomonadida, for example Histomonas spp., Pentatrichomonas spp., Tetratrichomonas spp., Trichomonas spp., Tritrichomonas spp.
Euglenozoa: from the order of Trypanosomatida, for example Leishmania spp., Trypanosoma spp.
Sarcomastigophora (Rhizopoda) such as Entamoebidae, for example, Entamoeba spp., Centramoebidae, for example Acanthamoeba sp., Euamoebidae, e.g. Hartmanella sp.
Alveolata such as Apicomplexa (Sporozoa): e.g. Cryptosporidium spp.; from the order of Eimeriida, for example, Besnoitia spp., Cystoisospora spp., Eimeria spp., Hammondia spp., Isospora spp., Neospora spp., Sarcocystis spp., Toxoplasma spp.; from the order of Adeleida, for example, Hepatozoon spp., Klossiella spp.; from the order of Haemosporida, for example, Leucocytozoon spp., Plasmodium spp.; from the order of Piroplasmida, for example, Babesia spp., Ciliophora spp., Echinozoon spp., Theileria spp.; from the order of Vesibuliferida, for example, Balantidium spp., Buxtonella spp.
Microspora such as Encephalitozoon spp., Enterocytozoon spp., Globidium spp., Nosema spp., and also, for example, Myxozoa spp.
The helminths that are pathogenic to humans or animals include, for example, Acanthocephala, nematodes, Pentastoma and Platyhelminthes (e.g. Monogenea, cestodes and trematodes).

Illustrative helminths include, but are not limited to:
Monogenea: e.g. Dactylogyrus spp., Gyrodactylus spp., Microbothrium spp., Polystoma spp., Troglecephalus spp.; Cestodes: from the order of Pseudophyllidea, for example: Bothridium spp., Diphyllobothrium spp., Diplogonoporus spp., Ichthyobothrium spp., Ligula spp., Schistocephalus spp., Spirometra spp. from the order of Cyclophyllida, for example: Andyra spp., Anoplocephala spp., Avitellina spp., Bertiella spp., Cittotaenia spp., Davainea spp., Diorchis spp., Diplopylidium spp., Dipylidium spp., Echinococcus spp., Echinocotyle spp., Echinolepis spp., Hydatigera spp., Hymenolepis spp., Joyeuxiella spp., Mesocestoides spp., Moniezia spp., Paranoplocephala spp., Raillietina spp., Stilesia spp., Taenia spp., Thysaniezia spp., Thysanosoma spp.
Trematodes: from the class of Digenea, for example: Austrobilharzia spp., Brachylaima spp., Calicophoron spp., Catatropis spp., Clonorchis spp. Collyriclum spp., Cotylophoron spp., Cyclocoelum spp., Dicrocoelium spp., Diplostomum spp., Echinochasmus spp., Echinoparyphium spp., Echinostoma spp., Eurytrema spp., Fasciola spp., Fasciolides spp., Fasciolopsis spp., Fischoederius spp., Gastrothylacus spp., Gigantobilharzia spp., Gigantocotyle spp., Heterophyes spp., Hypoderaeum spp., Leucochloridium spp., Metagonimus spp., Metorchis spp., Nanophyetus spp., Notocotylus spp., Opisthorchis spp., Ornithobilharzia spp., Paragonimus spp., Paramphistomum spp., Plagiorchis spp., Posthodiplostomum spp., Prosthogonimus spp., Schistosoma spp., Trichobilharzia spp., Troglotrema spp., Typhlocoelum spp.
Nematodes: from the order of Trichinellida, for example: Capillaria spp., Eucoleus spp., Paracapillaria spp., Trichinella spp., Trichomosoides spp., Trichuris spp.
From the order of Tylenchida, for example: Micronema spp., Parastrangyloides spp., Strongyloides spp.
From the order of Rhabditina, for example: Aelurostrongylus spp., Amidostomum spp., Ancylostoma spp., Angiostrongylus spp., Bronchonema spp., Bunostomum spp., Chabertia spp., Cooperia spp., Cooperioides spp., Crenosoma spp., Cyathostomum spp., Cyclococercus spp., Cyclodontostomum spp., Cylicocyclus spp., Cylicostephanus spp., Cylindropharynx spp., Cystocaulus spp., Dictyocaulus spp., Elaphostrongylus spp., Filaroides spp., Globocephalus spp., Graphidium spp., Gyalocephalus spp., Haemonchus spp., Heligmosomoides spp., Hyostrongylus spp., Marshallagia spp., Metastrongylus spp., Muellerius spp., Necator spp., Nematodirus spp., Neostrongylus spp., Nippostrongylus spp., Obeliscoides spp., Oesophagodontus spp., Oesophagostomum spp., Ollulanus spp.; Ornithostrongylus spp., Oslerus spp., Ostertagia spp., Paracooperia spp., Paracrenosoma spp., Parafilaroides spp., Parelaphostrongylus spp., Pneumocaulus spp., Pneumostrongylus spp., Poteriostomum spp., Protostrongylus spp., Spicocaulus spp., Stephanurus spp., Strongylus spp., Syngamus spp., Teladorsagia spp., Trichonema spp., Trichostrongylus spp., Triodontophorus spp., Troglostrongylus spp., Uncinaria spp.
From the order Spirurida, for example: Acanthocheilonema spp., Anisakis spp., Ascaridia spp.; Ascaris spp., Ascarops spp., Aspiculuris spp., Baylisascaris spp., Brugia spp., Cercopithifilaria spp., Crassicauda spp., Dipetalonema spp., Dirofilaria spp., Dracunculus spp.; Draschia spp., Enterobius spp., Filaria spp., Gnathostoma spp., Gongylonema spp., Habronema spp., Heterakis spp.; Litomosoides spp., Loa spp., Onchocerca spp., Oxyuris spp., Parabronema spp., Parafilaria spp., Parascaris spp., Passalurus spp., Physaloptera spp., Probstmayria spp., Pseudofilaria spp., Setaria spp., Skjrabinema spp., Spirocerca spp., Stephanofilaria spp., Strongyluris spp., Syphacia spp., Thelazia spp., Toxascaris spp., Toxocara spp., Wuchereria spp.
Acanthocephala: from the order of Oligacanthorhynchida, for example: Macracanthorhynchus spp., Prosthenorchis spp.; from the order of Moniliformida, for example: Moniliformis spp.
From the order of Polymorphida, for example: Filicollis spp.; from the order of Echinorhynchida, for example Acanthocephalus spp., Echinorhynchus spp., Leptorhynchoides spp.
Pentastoma: from the order of Porocephalida, for example Linguatula spp.

In the veterinary field and in animal husbandry, the compounds of the formula (I) are administered by methods generally known in the art, such as via the enteral, parenteral, dermal or nasal route in the form of suitable preparations. Administration may be prophylactic, metaphylactic or therapeutic.

Thus, one embodiment of the present invention refers to the compounds of the formula (I) for use as a medicament.

A further aspect relates to the compounds of the formula (I) for use as an antiendoparasitic agent.

A further specific aspect relates to the compounds of formula (I) for use as an antihelminthic agent, especially for use as a nematicide, platyhelminthicide, acanthocephalicide or pentastomicide.

A further specific aspect relates to the compounds of the formula (I) for use as an antiprotozoic agent.

A further aspect relates to the compounds of the formula (I) for use as an antiectoparasitic agent, especially an arthropodicide, very particularly an insecticide or an acaricide.

Further aspects of the invention are veterinary medicine formulations comprising an effective amount of at least one compound of the formula (I) and at least one of the following: a pharmaceutically acceptable excipient (e.g. solid or liquid diluents), a pharmaceutically acceptable auxiliary (e.g. surfactants), especially a pharmaceutically acceptable excipient used conventionally in veterinary medicine formulations and/or a pharmaceutically acceptable auxiliary conventionally used in veterinary medicine formulations.

A related aspect of the invention is a method for production of a veterinary medicine formulation as described here, which comprises the step of mixing at least one compound of the formula (I) with pharmaceutically acceptable excipients and/or auxiliaries, especially with pharmaceutically acceptable excipients used conventionally in veterinary medicine formulations and/or auxiliaries.

Another specific aspect of the invention is veterinary medicine formulations selected from the group of ectoparasiticidal and endoparasiticidal formulations, especially selected from the group of anthelmintic, antiprotozoic and arthropodicidal formulations, very particularly selected from the group of nematicidal, platyhelminthicidal, acanthocephalicidal, pentastomicidal, insecticidal and acaricidal formulations, according to the aspects mentioned, and methods for production thereof.

Another aspect relates to a method for treatment of a parasitic infection, especially an infection caused by a parasite selected from the group of the ectoparasites and endoparasites mentioned here, by use of an effective amount of a compound of the formula (I) in an animal, especially a nonhuman animal, having a need therefor.

Another aspect relates to a method for treatment of a parasitic infection, especially an infection caused by a parasite selected from the group of the ectoparasites and endoparasites mentioned here, by use of a veterinary medicine formulation as defined here in an animal, especially a nonhuman animal, having a need therefor.

Another aspect relates to the use of the compounds of the formula (I) in the treatment of a parasite infection, especially an infection caused by a parasite selected from the group of the ectoparasites and endoparasites mentioned here, in an animal, especially a nonhuman animal.

In the present context of animal health or veterinary medicine, the term “treatment” includes prophylactic, metaphylactic and therapeutic treatment.

In a particular embodiment, in this way, mixtures of at least one compound of the formula (I) with other active ingredients, especially with endo- and ectoparasiticides, are provided for the field of veterinary medicine.

In the field of animal health, “mixture” means not just that two (or more) different active ingredients are formulated in a common formulation and are correspondingly employed together, but also relates to products comprising formulations separated for each active ingredient. Accordingly, when more than two active ingredients are to be employed, all active ingredients can be formulated in a common formulation or all active ingredients can be formulated in separate formulations; likewise conceivable are mixed forms in which some of the active ingredients are formulated together and some of the active ingredients are formulated separately. Separate formulations allow the separate or successive application of the active ingredients in question.

The active ingredients specified here by their “common names” are known and are described, for example, in the “Pesticide Manual” (see above) or can be searched for on the Internet (e.g.: http://www.alanwood.net/pesticides).

Illustrative active ingredients from the group of the ectoparasiticides as mixing components, without any intention that this should constitute a restriction, include the insecticides and acaricides listed in detail above. Further usable active ingredients are listed below in accordance with the abovementioned classification based on the current IRAC Mode of Action Classification Scheme: (1) acetylcholinesterase (AChE) inhibitors; (2) GABA-gated chloride channel blockers; (3) sodium channel modulators; (4) nicotinic acetylcholine receptor (nAChR) competitive modulators; (5) nicotinic acetylcholine receptor (nAChR) allosteric modulators; (6) glutamate-gated chloride channel (GluCl) allosteric modulators; (7) juvenile hormone mimetics; (8) miscellaneous non-specific (multi-site) inhibitors; (9) chordotonal organ modulators; (10) mite growth inhibitors; (12) inhibitors of mitochondrial ATP synthase, such as ATP disruptors; (13) uncouplers of oxidative phosphorylation via disruption of the proton gradient; (14) nicotinic acetylcholine receptor channel blockers; (15) inhibitors of chitin biosynthesis, type 0; (16) inhibitors of chitin biosynthesis, type 1; (17) moulting disruptors (especially in Diptera); (18) ecdysone receptor agonists; (19) octopamine receptor agonists; (21) mitochondrial complex I electron transport inhibitors; (25) mitochondrial complex II electron transport inhibitors; (20) mitochondrial complex III electron transport inhibitors; (22) voltage-dependent sodium channel blockers; (23) inhibitors of acetyl CoA carboxylase; (28) ryanodine receptor modulators; (30) allosteric modulators of the GABA-dependent chloride channel.
active ingredients having unknown or non-specific mechanisms of action, e.g. fentrifanil, fenoxacrim, cycloprene, chlorobenzilate, chlordimeform, flubenzimin, dicyclanil, amidoflumet, quinomethionate, triarathene, clothiazoben, tetrasul, potassium oleate, petroleum, metoxadiazone, gossyplur, flutenzine, bromopropylate, cryolite;

Compounds from other classes:
butacarb, dimetilan, cloethocarb, phosphocarb, pirimiphos(-ethyl), parathion(-ethyl), methacrifos, isopropyl o-salicylate, trichlorfon, sulprofos, propaphos, sebufos, pyridathion, prothoate, dichlofenthion, demeton-S-methyl sulfone, isazofos, cyanofenphos, dialifos, carbophenothion, autathiofos, aromfenvinfos(-methyl), azinphos(-ethyl), chlorpyrifos(-ethyl), fosmethilan, iodofenphos, dioxabenzofos, formothion, fonofos, flupyrazofos, fensulfothion, etrimfos;
organochlorine compounds e.g. camphechlor, lindane, heptachlor;
phenylpyrazoles, e.g. acetoprol, pyrafluprol, pyriprol, vaniliprol, sisapronil;
isoxazolines, e.g. afoxolaner, lotilaner, fluralaner, sarolaner;
pyrazolyl-arylamides, e.g. nicofluprol, tigolaner;
pyrethroids, e.g. (cis-, trans-)metofluthrin, profluthrin, flufenprox, flubrocythrinate, fubfenprox, fenfluthrin, protrifenbut, pyresmethrin, RU15525, terallethrin, cis-resmethrin, heptafluthrin, bioethanomethrin, biopermethrin, fenpyrithrin, cis-cypermethrin, cis-permethrin, clocythrin, cyhalothrin (lambda-), chlovaporthrin, or halogenated hydrocarbon compounds (HCHs);
neonicotinoids, e.g. nithiazine;
dicloromezotiaz, triflumezopyrim;
macrocyclic lactones, e.g. nemadectin, ivermectin, latidectin, moxidectin, selamectin, eprinomectin, doramectin, emamectin benzoate, milbemycin oxime;
triprene, epofenonane, diofenolan;
biologicals, hormones or pheromones, for example natural products, e.g. thuringiensin, codlemone or neem components;
dinitrophenols, e.g. dinocap, dinobuton, binapacryl;
benzoylureas, e.g. fluazuron, penfluron;
amidine derivatives, e.g. chlormebuform, cymiazole, demiditraz;
beehive varroa acaricides, for example organic acids, e.g. formic acid, oxalic acid.

Illustrative active ingredients from the group of the endoparasiticides, as mixing components, include, but are not limited to, active anthelmintic ingredients and active antiprotozoic ingredients.

The active anthelmintic ingredients include, but are not limited to, the following active nematicidal, trematicidal and/or cestocidal ingredients:
from the class of the macrocyclic lactones, for example: eprinomectin, abamectin, nemadectin, moxidectin, doramectin, selamectin, lepimectin, latidectin, milbemectin, ivermectin, emamectin, milbemycin;
from the class of the benzimidazoles and probenzimidazoles, for example: oxibendazole, mebendazole, triclabendazole, thiophanate, parbendazole, oxfendazole, netobimin, fenbendazole, febantel, thiabendazole, cyclobendazole, cambendazole, albendazole sulfoxide, albendazole, flubendazole;
from the class of the depsipeptides, preferably cyclic depsipeptides, especially 24-membered cyclic depsipeptides, for example: emodepside, PF1022A;
from the class of the tetrahydropyrimidines, for example: morantel, pyrantel, oxantel; from the class of the imidazothiazoles, for example: butamisole, levamisole, tetramisole;
from the class of the aminophenylamidines, for example: amidantel, deacylated amidantel (dAMD), tribendimidine;
from the class of the aminoacetonitriles, for example: monepantel;
from the class of the paraherquamides, for example: paraherquamide, derquantel;
from the class of the salicylanilides, for example: tribromsalan, bromoxanide, brotianide, clioxanide, closantel, niclosamide, oxyclozanide, rafoxanide;
from the class of the substituted phenols, for example: nitroxynil, bithionol, disophenol, hexachlorophene, niclofolan, meniclopholan;
from the class of the organophosphates, for example: trichlorfon, naphthalofos, dichlorvos/DDVP, crufomate, coumaphos, haloxon;
from the class of the piperazinones/quinolines, for example: praziquantel, epsiprantel;
from the class of the piperazines, for example: piperazine, hydroxyzine;
from the class of the tetracyclines, for example: tetracycline, chlorotetracycline, doxycycline, oxytetracycline, rolitetracycline;
from various other classes, for example: bunamidine, niridazole, resorantel, omphalotin, oltipraz, nitroscanate, nitroxynil, oxamniquin, mirasan, miracil, lucanthon, hycanthon, hetolin, emetin, diethylcarbamazine, dichlorophen, diamfenetide, clonazepam, bephenium, amoscanate, clorsulon.

Active antiprotozoic ingredients include, but are not limited to, the following active ingredients:
from the class of the triazines, for example: diclazuril, ponazuril, letrazuril, toltrazuril;
from the class of polyether ionophores, for example: monensin, salinomycin, maduramicin, narasin;
from the class of the macrocyclic lactones, for example: milbemycin, erythromycin;
from the class of the quinolones, for example: enrofloxacin, pradofloxacin;
from the class of the quinines, for example: chloroquine;
from the class of the pyrimidines, for example: pyrimethamine;
from the class of the sulfonamides, for example: sulfaquinoxaline, trimethoprim, sulfaclozin;
from the class of the thiamines, for example: amprolium;
from the class of the lincosamides, for example: clindamycin;
from the class of the carbanilides, for example: imidocarb;
from the class of the nitrofurans, for example: nifurtimox;
from the class of the quinazolinone alkaloids, for example: halofuginone;
from various other classes, for example: oxamniquine, paromomycin;
from the class of the vaccines or antigens from microorganisms, for example: Babesia canis rossi, Eimeria tenella, Eimeria praecox, Eimeria necatrix, Eimeria mitis, Eimeria maxima, Eimeria brunetti, Eimeria acervulina, Babesia canis vogeli, Leishmania infantum, Babesia canis canis, Dictyocaulus viviparus.

All the mixing components mentioned, as the case may be, may also form salts with suitable bases or acids if they are capable of doing so on the basis of their functional groups.

### Vector Control

The compounds of formula (I) can also be used in vector control. In the context of the present invention, a vector is an arthropod, especially an insect or arachnid, capable of transmitting pathogens, for example viruses, worms, single-cell organisms and bacteria, from a reservoir (plant, animal, human, etc.) to a host. The pathogens can be transmitted either mechanically (for example trachoma by non-stinging flies) onto a host or after injection into a host (for example malaria parasites by mosquitoes).

Examples of vectors and the diseases or pathogens they transmit are:
1) Mosquitoes
Anopheles: malaria, filariasis;
Culex: Japanese encephalitis, other viral diseases, filariasis, transmission of other worms;
Aedes: yellow fever, dengue fever, other viral diseases, filariasis;
Simuliidae: transmission of worms, especially Onchocerca volvulus;
Psychodidae: transmission of leishmaniasis
2) Lice: skin infections, epidemic typhus;
3) Fleas: plague, endemic typhus, tapeworms;
4) Flies: sleeping sickness (trypanosomiasis); cholera, other bacterial diseases;
5) Mites: acariosis, epidemic typhus, rickettsialpox, tularaemia, Saint Louis encephalitis, tick-borne encephalitis (TBE), Crimean-Congo haemorrhagic fever, borreliosis;
6) Ticks: borrelioses such as Borrelia bungdorferi sensu lato., Borrelia duttoni, tick-borne encephalitis, Q fever (Coxiella burnetii), babesioses (Babesia canis canis), ehrlichiosis.

Examples of vectors in the context of the present invention are insects, for example aphids, flies, leafhoppers or thrips, which can transmit plant viruses to plants. Other vectors capable of transmitting plant viruses are spider mites, lice, beetles and nematodes.

Further examples of vectors in the context of the present invention are insects and arachnids such as mosquitoes, especially of the genera Aedes, Anopheles, for example A. gambiae, A. arabiensis, A. funestus, A. dirus (malaria) and Culex, Psychodidae such as Phlebotomus, Lutzomyia, lice, fleas, flies, mites and ticks, which can transmit pathogens to animals and/or humans.

Vector control is also possible if the compounds of the formula (I) are resistance-breaking.

Compounds of the formula (I) are suitable for use in the prevention of diseases and/or pathogens transmitted by vectors. Thus, a further aspect of the present invention is the use of compounds of formula (I) for vector control, for example in agriculture, in horticulture, in gardens and in leisure facilities, and also in the protection of materials and stored products.

### Protection of Industrial Materials

The compounds of the formula (I) are suitable for protecting industrial materials against attack or destruction by insects, for example from the orders of Coleoptera, Hymenoptera, Isoptera, Lepidoptera, Psocoptera and Zygentoma.

Industrial materials in the present context are understood to mean inanimate materials, such as preferably plastics, adhesives, sizes, papers and cards, leather, wood, processed wood products and coating compositions. The use of the invention for protection of wood is particularly preferred.

In a further embodiment, the compounds of the formula (I) are used together with at least one further insecticide and/or at least one fungicide.

In a further embodiment, the compounds of the formula (I) take the form of a ready-to-use pesticide, meaning that they can be applied to the material in question without further modifications. Useful further insecticides or fungicides especially include those mentioned above.

Surprisingly, it has also been found that the compounds of formula (I) can be employed for protecting objects which come into contact with saltwater or brackish water, in particular hulls, screens, nets, buildings, moorings and signalling systems, against fouling. It is equally possible to use the compounds of the formula (I), alone or in combinations with other active ingredients, as antifouling agents.

### Control of Animal Pests in the Hygiene Sector

The compounds of the formula (I) are suitable for controlling animal pests in the hygiene sector. More particularly, the invention can be used in the domestic protection sector, in the hygiene protection sector and in the protection of stored products, particularly for control of insects, arachnids, ticks and mites encountered in enclosed spaces, for example dwellings, factory halls, offices, vehicle cabins and animal breeding facilities. For controlling animal pests, the compounds of the formula (I) are used alone or in combination with other active ingredients and/or auxiliaries. They are preferably used in domestic insecticide products. The compounds of the formula (I) are effective against sensitive and resistant species, and against all developmental stages.

These pests include, for example, pests from the class Arachnida, from the orders Scorpiones, Araneae and Opiliones, from the classes Chilopoda and Diplopoda, from the class Insecta the order Blattodea, from the orders Coleoptera, Dermaptera, Diptera, Heteroptera, Hymenoptera, Isoptera, Lepidoptera, Phthiraptera, Psocoptera, Saltatoria or Orthoptera, Siphonaptera and Zygentoma and from the class Malacostraca the order Isopoda.

Application is effected, for example, in aerosols, unpressurized spray products, for example pump and atomizer sprays, automatic fogging systems, foggers, foams, gels, evaporator products with evaporator tablets made of cellulose or plastic, liquid evaporators, gel and membrane evaporators, propeller-driven evaporators, energy-free, or passive, evaporation systems, moth papers, moth bags and moth gels, as granules or dusts, in baits for spreading or bait stations.

### Analytical Methods

The procedures described below for the analytical methods relate to all statements throughout the document, unless the procedure for the respective analytical determination method is described separately in the relevant text passage.

### Mass Spectrometry

The determination of [M+H]⁺ or M⁻ by LC-MS under acidic chromatographic conditions was carried out using 1 ml of formic acid per liter of acetonitrile and 0.9 ml of formic acid per liter of Millipore water as eluents. The Zorbax Eclipse Plus C18 column, 50 mm*2.1 mm, was used, at a column oven temperature of 55° C.

Instruments:

LC-MS3: Waters UPLC with SQD2 mass spectrometer and SampleManager sample changer. Linear gradient from 0.0 to 1.70 minutes from 10% acetonitrile to 95% acetonitrile, from 1.70 to 2.40 minutes constant 95% acetonitrile, flow rate 0.85 ml/min.

LC-MS6 and LC-MS7: Agilent 1290 LC, Agilent MSD, HTS PAL sample changer. Linear gradient from 0.0 to 1.80 minutes from 10% acetonitrile to 95% acetonitrile, from 1.80 to 2.50 minutes constant 95% acetonitrile, flow rate 1.0 ml/min.

The determination of [M+H]⁺ by LC-MS under neutral chromatographic conditions was carried out using acetonitrile and Millipore water with 79 mg/l ammonium carbonate as eluents.

Instruments:

LC-MS4: Waters IClass Acquity with QDA mass spectrometer and FTN sample changer (column Waters Acquity 1.7 μm 50 mm*2.1 mm, oven temperature 45° C.). Linear gradient from 0.0 to 2.10 minutes from 10% acetonitrile to 95% acetonitrile, from 2.10 to 3.00 minutes constant 95% acetonitrile, flow rate 0.7 ml/min.

LC-MS8: Waters IClass Acquity with QDA mass spectrometer and FTN sample changer (column Waters Acquity 1.7 μm 50 mm*2.1 mm, oven temperature 45° C.). Linear gradient from 0.0 to 2.10 minutes from 10% acetonitrile to 95% acetonitrile, from 2.10 to 3.00 minutes constant 95% acetonitrile, flow rate 0.7 ml/min.

In all cases, the retention time indices were determined according to a homologous series of straight-chain alkan-2-ones having 3 to 16 carbons, where the index of the first alkanone was set to 300, the index of the last alkanone was set to 1600 and linear interpolation was carried out between the values of successive alkanones.

The ¹H NMR spectra were measured with a Bruker Avance III 400 MHz spectrometer fitted with a 1.7 mm TCI sample head using tetramethylsilane as standard (0.00 ppm), and the measurements were generally recorded of solutions in the solvents CD₃CN, CDCl₃ or d₆-DMSO. Alternatively, a Bruker Avance III 600 MHz spectrometer fitted with a 5 mm CPNMP sample head or a Bruker Avance NEO 600 MHz spectrometer fitted with a 5 mm TCI sample head was employed for the measurements. In general, the measurements were carried out at a sample head temperature of 298 K. If other measurement temperatures were used, this is specifically mentioned.

### NMR Peak List Method

The ¹H NMR data of selected examples are represented in the form of ¹H NMR peak lists. For each signal peak, first the a value in ppm and then the signal intensity in round brackets are listed. The a value/signal intensity number pairs are listed with separation from one another by semicolons.

The peak list for one example therefore has the form:

δ₁(intensity₁); δ₂ (intensity₂); . . . ; δᵢ (intensityᵢ); . . . ; Sₙ (intensityₙ)

The intensity of sharp signals correlates with the height of the signals in a printed representation of a ¹H NMR spectrum in cm and shows the true ratios of the signal intensities. In the case of broad signals, several peaks or the middle of the signal and the relative intensity thereof may be shown in comparison to the most intense signal in the spectrum.

Calibration of the chemical shift of ¹H NMR spectra is accomplished using tetramethylsilane or the chemical shift of the solvent if the sample does not contain any tetramethylsilane. Accordingly, in certain cases the ¹H NMR peak lists may comprise the tetramethylsilane peak.

The ¹H NMR peak lists are equivalent to conventional ¹H NMR representations and thus usually contain all peaks also listed in conventional ¹H NMR interpretations.

In addition, like conventional ¹H NMR representations, they may show solvent signals, signals of stereoisomers of the compounds which are optionally provided by the invention, and/or peaks of impurities.

¹H NMR solvent signals, the tetramethylsilane signal and the water signal in the solvent in question are excluded from the calibration of the relative intensity since their stated intensity values can be very high.

The peaks of stereoisomers of the compounds of the invention and/or peaks of impurities usually have a lower intensity than the peaks of the compounds of the invention (for example at a purity of >90%).

Such stereoisomers and/or impurities may be typical of the particular preparation process. Their peaks can thus help in this case to identify reproduction of a preparation process with reference to “by-product fingerprints”.

An expert calculating the peaks of the target compounds by known methods (MestreC, ACD simulation, but also with empirically evaluated expected values) can, if required, identify the peaks of the target compounds, optionally using additional intensity filters. This identification is equivalent to the relevant peak listing in conventional ¹H NMR interpretation.

The solvent utilized can be read off from the JCAMP file from the parameter “solvent”, the measurement frequency of the spectrometer from “observe frequency”, and the spectrometer model from “spectrometer/data system”.

¹³C NMR data are stated analogously to the ¹H NMR data as peak lists using broadband-decoupled ¹³C NMR spectra. ¹³C NMR solvent signals and tetramethylsilane are excluded from the calibration of the relative intensity since these signals may have very high intensity values.

Further details of NMR data description using peak lists can be found in: “Citation of NMR Peaklist Data within Patent Applications” in Research Disclosure Database Number 564025.

### log P Values

The log P values were determined according to EEC Directive 79/831 Annex V.A8 by HPLC (high-performance liquid chromatography) on a reversed-phase column (C18) using the following methods:

^{[a]} The log P value is determined by LC-UV measurement in the acidic range using 0.9 ml/l formic acid in water and 1.0 ml/l formic acid in acetonitrile as eluents (linear gradient from 10% acetonitrile to 95% acetonitrile).

^{[b]} The log P value is determined by LC-UV measurement in the neutral range using 0.001 molar ammonium acetate solution in water and acetonitrile as eluents (linear gradient from 10% acetonitrile to 95% acetonitrile).

Calibration was carried out using straight-chain alkan-2-ones (having 3 to 16 carbon atoms) with known log P values. The values between successive alkanones are determined by linear regression.

### Preparation Examples

### Example I-02

### 2-[5-Ethylsulfonyl-6-[3-methyl-6-(1,1,2,2,2-pentafluoroethyl)imidazo[4,5-b]pyridin-2-yl]-2-pyridyl]-4-(4-fluorophenyl)-1,2,4-triazol-3-one

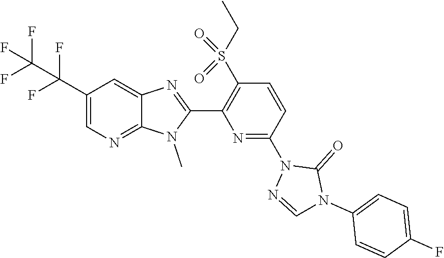 [SMILES: CCS(=O)(=O)c1ccc(-n2ncn(-c3ccc(F)cc3)c2=O)nc1-c1nc2cc(C(F)(F)C(F)(F)F)cnc2n1C]

59 mg (0.13 mmol) of 2-(3-ethylsulfonyl-6-fluoro-2-pyridyl)-3-methyl-6-(1,1,2,2,2-pentafluoroethyl)imidazo[4,5-b]pyridine was dissolved in 6 ml of acetonitrile, 66.4 mg (0.20 mmol) of caesium carbonate, 11.4 mg (0.06 mmol) of potassium iodide and 38.1 mg (0.20 mmol) of 4-(4-fluorophenyl)-1H-1,2,4-triazol-5-one were added, and the mixture was stirred at room temperature for 20 h. Subsequently, the reaction mixture was filtered, and the filtrate was freed of the solvent under reduced pressure.

The residue was dissolved in 2 ml of dichloromethane and purified by column chromatography purification with a cyclohexane/ethyl acetate solvent mixture (2:1) as eluent.

log P (neutral): 3.53; MH⁺: 598; ¹H-NMR (400 MHz, D6-DMSO) δ ppm: 1.25 (t, 3H), 3.88 (q, 2H), 3.91 (s, 3H), 7.42-7.46 (m, 2H), 7.75-7.79 (m, 2H), 8.50 (d, 1H), 8.67-8.72 (m, 2H), 8.81 (s, 1H), 8.85-8.86 (m, 1H).

### 2-(3-Ethylsulfonyl-6-fluoro-2-pyridyl)-3-methyl-6-(1,1,2,2,2-pentafluoroethyl)imidazo[4,5-b]pyridine

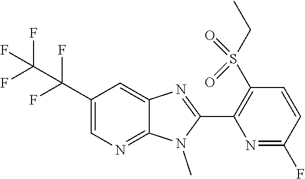 [SMILES: CCS(=O)(=O)c1ccc(F)nc1-c1nc2cc(C(F)(F)C(F)(F)F)cnc2n1C]

2.20 g (5.41 mmol) of 2-(3-ethylsulfanyl-6-fluoro-2-pyridyl)-3-methyl-6-(1,1,2,2,2-pentafluoroethyl)imidazo[4,5-b]pyridine was dissolved in 120 ml of dichloromethane, 2.44 g (53.0 mmol) of formic acid and 4.87 g (50.0 mmol) of 35% hydrogen peroxide were added at room temperature, and then the mixture was stirred at room temperature for 17 h. The mixture was diluted with 20 ml of water, 3 ml of sodium bisulfite solution was added, the mixture was stirred for 30 min, and then saturated sodium carbonate solution was added. The organic phase was separated off, the aqueous phase was extracted twice with dichloromethane, and then the combined organic phases were freed of the solvent under reduced pressure. The residue was used in the next stage without further purification.

log P (neutral): 3.33; MH⁺: 439; ¹H-NMR (400 MHz, D₆-DMSO) δ ppm: 1.22 (t, 3H), 3.79-3.85 (m, 5H), 7.79-7.82 (m, 1H), 8.67 (d, 1H), 8.70-8.74 (m, 1H), 8.85 (d, 1H).

### 2-(3-Ethylsulfanyl-6-fluoro-2-pyridyl)-3-methyl-6-(1,1,2,2,2-pentafluoroethyl)imidazo[4,5-b]pyridine

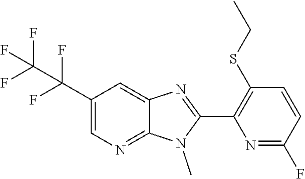 [SMILES: CCSc1ccc(F)nc1-c1nc2cc(C(F)(F)C(F)(F)F)cnc2n1C]

2.54 g (6.97 mmol) of 2-(3,6-difluoro-2-pyridyl)-3-methyl-6-(1,1,2,2,2-pentafluoroethyl)imidazo[4,5-b]pyridine was dissolved in 32 ml of tetrahydrofuran, the mixture was cooled to −10° C., and 285 mg (7.11 mmol) of sodium hydride was added. Stirring was continued at −10 to −15° C. for a further 15 min, and then 477 mg (7.67 mmol) of ethanethiol, dissolved in 5 ml of tetrahydrofuran, was added dropwise over the course of 50 minutes. The mixture was stirred at −10 to −5° C. for a further 3 h and then poured onto ice-water, and the precipitated solids were filtered off. The residue was purified by column chromatography purification by means of preparative HPLC with a water/acetonitrile gradient as eluent.

log P (acidic): 4.05; MH⁺: 407; ¹H-NMR (400 MHz, D₆-DMSO) δ ppm: 1.20 (t, 3H), 3.03 (q, 2H), 3.96 (s, 3H), 7.48-7.51 (m, 1H), 8.27-8.31 (m, 1H), 8.63 (d, 1H), 8.81 (d, 1H).

### 2-(3,6-Difluoro-2-pyridyl)-3-methyl-6-(1,1,2,2,2-pentafluoroethyl)imidazo[4,5-b]pyridine

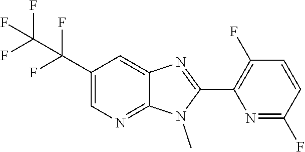 [SMILES: Cn1c(-c2nc(F)ccc2F)nc2cc(C(F)(F)C(F)(F)F)cnc21]

3.50 g (13.7 mmol) of N2-methyl-5-(1,1,2,2,2-pentafluoroethyl)pyridine-2,3-diamine, 2.83 g (17.2 mmol) of 3,6-difluoropyridine-2-carboxylic acid and 4.01 g (20.6 mmol) of 1-(3-dimethylaminopropyl)-3-ethylcarbodiimide hydrochloride (EDCI) were stirred in 60 ml of pyridine at room temperature for 16 h. The reaction mixture was freed of the solvent under reduced pressure, and the residue was taken up in 80 ml of glacial acetic acid and stirred under reflux for 6 h. Subsequently, the solvent was distilled off under reduced pressure and the residue was partitioned between dichloromethane and a semisaturated sodium hydrogencarbonate solution.

The organic phase was removed, dried over sodium sulfate and concentrated. The residue was admixed with 10 ml of methyl tert-butyl ether and stirred. The remaining residue was finally filtered off and dried.

log P (neutral): 3.30; MH⁺: 365; ¹H-NMR (400 MHz, D₆-DMSO) δ ppm: 4.07 (s, 3H), 7.59-7.63 (m, 1H), 8.27-8.33 (m, 1H), 8.67 (d, 1H), 8.82 (d, 1H).

In analogy to the examples and according to the above-described preparation processes, the following compounds of the formula (I) can be obtained:

| Ex. | Structure | |
|---|---|---|
| I-01 | 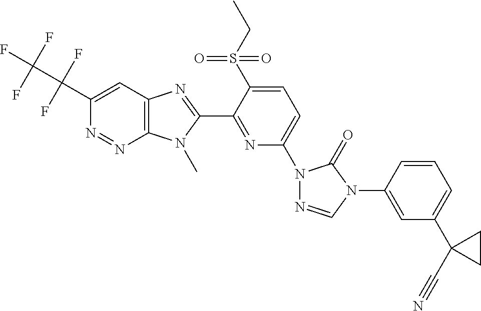 [SMILES: CCS(=O)(=O)c1ccc(-n2ncn(-c3cccc(C4(C#N)CC4)c3)c2=O)nc1-c1nc2cc(C(F)(F)C(F)(F)F)nnc2n1C] | : ¹H-NMR(400.2 MHz, d₆-DMSO): δ = 8.8988 (8.3); 8.8587 (7.0); 8.8330 (0.8); 8.7547 (3.1); 8.7325 (4.2); 8.5943 (4.2); 8.5720 (3.6); 8.4617 (0.4); 8.3684 (0.4); 8.3158 (1.4); 7.7009 (1.1); 7.6986 (1.0); 7.6810 (2.0); 7.6787 (1.7); 7.6706 (2.1); 7.6663 (3.0); 7.6617 (1.4); 7.6204 (1.5); 7.6005 (2.6); 7.5807 (1.3); 7.5547 (0.4); 7.4552 (1.6); 7.4355 (1.2); 4.0645 (16.0); 3.9906 (1.9); 3.8248 (0.9); 3.8065 (3.2); 3.7881 (3.2); 3.7698 (0.9); 3.6532 (0.4); 3.6340 (0.4); 3.3303 (680.0); 2.6758 (2.6); 2.6713 (3.5); 2.6667 (2.5); 2.6623 (1.2); 2.5947 (0.3); 2.5247 (12.4); 2.5199 (19.3); 2.5113 (215.9); 2.5069 (423.1); 2.5023 (543.5); 2.4977 (391.0); 2.4933 (189.0); 2.3382 (1.2); 2.3337 (2.5); 2.3291 (3.3); 2.3246 (2.4); 2.3202 (1.2); 1.8526 (1.1); 1.8400 (3.7); 1.8328 (3.8); 1.8213 (1.5); 1.7563 (0.5); 1.7499 (0.4); 1.6264 |
| | | (1.5); 1.6138 (3.6); 1.6067 (3.8); 1.5933 |
| | | (1.1); 1.4683 (0.4); 1.4619 (0.4); 1.2781 |
| | | (0.5); 1.2622 (3.5); 1.2437 (8.2); 1.2252 |
| | | (3.5); 1.2170 (0.7); 1.1981 (1.0); 1.1800 |
| | | (0.4); 0.1460 (1.2); 0.0079 (10.8); −0.0002 |
| | | (300.2); −0.0085 (10.7); −0.1496 (1.2) |
| I-02 | 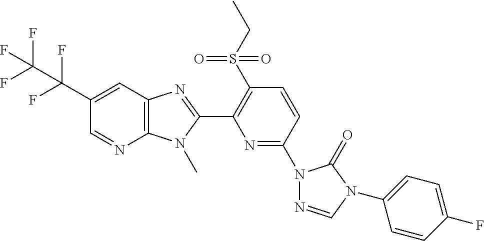 [SMILES: CCS(=O)(=O)c1ccc(-n2ncn(-c3ccc(F)cc3)c2=O)nc1-c1nc2cc(C(F)(F)C(F)(F)F)cnc2n1C] | : ¹H-NMR(400.2 MHz, d₆-DMSO): δ = 8.8553 (2.2); 8.8505 (2.2); 8.8188 (8.5); 8.7196 (3.2); 8.6975 (4.1); 8.6783 (2.3); 8.6735 (2.2); 8.5145 (4.1); 8.4924 (3.6); 7.7876 (2.1); 7.7819 (0.8); 7.7755 (2.3); 7.7702 (1.3); 7.7648 (2.6); 7.7584 (0.8); 7.7527 (2.4); 7.4612 (2.5); 7.4555 (0.7); 7.4486 (0.3); 7.4440 (0.9); 7.4392 (3.9); 7.4342 (0.8); 7.4227 (0.7); 7.4170 (2.2); 3.9085 (16.0); 3.8912 (3.0); 3.8727 (2.9); 3.8543 (0.8); 3.3285 (141.7); 2.8913 (0.7); 2.7326 (0.6); 2.6762 (0.5); 2.6716 (0.7); 2.6671 (0.5); 2.5252 (2.2); 2.5205 (3.2); 2.5118 (43.7); 2.5073 (89.0); 2.5027 (115.5); 2.4981 (80.7); 2.4935 (37.4); 2.3341 (0.5); 2.3295 (0.7); 2.3250 (0.5); |
| | | 2.0864 (1.8); 1.2660 (3.2); 1.2475 (7.6); |
| | | 1.2290 (3.2); 1.0692 (0.5); 0.0080 (0.5); |
| | | −0.0002 (16.5); −0.0085 (0.5) |
| I-03 | 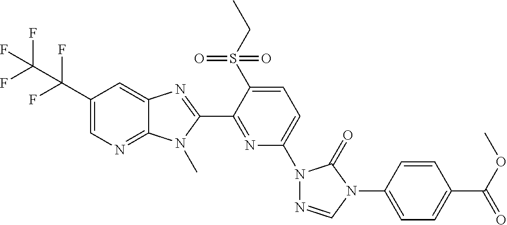 [SMILES: CCS(=O)(=O)c1ccc(-n2ncn(-c3ccc(C(=O)OC)cc3)c2=O)nc1-c1nc2cc(C(F)(F)C(F)(F)F)cnc2n1C] | : ¹H-NMR(400.2 MHz, d₆-DMSO): δ = 8.9956 (7.5); 8.8573 (2.5); 8.8528 (2.6); 8.7293 (2.9); 8.7071 (3.7); 8.6814 (2.7); 8.6769 (2.6); 8.5158 (3.7); 8.4937 (3.2); 8.1555 (3.9); 8.1511 (1.4); 8.1384 (1.6); 8.1337 (5.1); 7.9695 (4.9); 7.9650 (1.6); 7.9477 (4.1); 4.3690 (0.6); 4.3564 (1.3); 4.3437 (0.7); 3.9140 (16.0); 3.8920 (15.0); 3.8778 (3.4); 3.8593 (1.0); 3.4741 (0.3); 3.4613 (0.4); 3.4566 (1.1); 3.4439 (1.1); 3.4392 (1.1); 3.4265 (1.1); 3.4217 (0.4); 3.4090 (0.4); 3.3339 (48.4); 2.6725 (0.4); 2.5259 (1.3); 2.5120 (24.9); 2.5082 (48.4); 2.5037 (62.2); 2.4992 (45.3); 2.4950 (22.6); 2.3305 (0.4); 1.2687 (3.4); |
| | | 1.2503 (7.7); 1.2317 (3.4); 1.0741 (2.4); |
| | | 1.0567 (4.7); 1.0392 (2.3); 0.0078 (1.5); |
| | | −0.0002 (41.7); −0.0085 (1.6) |
| I-04 | 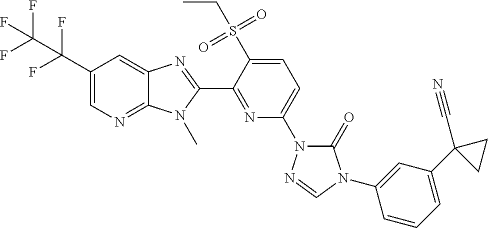 [SMILES: CCS(=O)(=O)c1ccc(-n2ncn(-c3cccc(C4(C#N)CC4)c3)c2=O)nc1-c1nc2cc(C(F)(F)C(F)(F)F)cnc2n1C] | : ¹H-NMR(400.2 MHz, d₆-DMSO): δ = 8.8884 (7.6); 8.8566 (2.4); 8.8521 (2.5); 8.7187 (3.0); 8.6966 (3.9); 8.6804 (2.5); 8.6757 (2.4); 8.5331 (3.8); 8.5109 (3.3); 8.3157 (3.1); 7.7016 (0.9); 7.6991 (1.0); 7.6968 (1.0); 7.6791 (1.8); 7.6769 (1.6); 7.6643 (1.9); 7.6598 (2.9); 7.6554 (1.5); 7.6167 (1.4); 7.5971 (2.6); 7.5772 (1.3); 7.4557 (1.5); 7.4360 (1.2); 7.4318 (1.0); 3.9018 (16.0); 3.8842 (3.1); 3.8657 (3.1); 3.8472 (0.9); 3.3247 (92.8); 3.3009 (1.7); 2.6757 (0.7); 2.6711 (0.9); 2.6667 (0.6); 2.5245 (3.2); 2.5111 (53.9); 2.5067 (106.6); 2.5022 (138.3); 2.4977 (98.9); 2.4932 (47.7); 2.3335 (0.6); 2.3290 (0.8); |
| | | 2.3245 (0.6); 1.8501 (1.2); 1.8373 (3.6); |
| | | 1.8302 (3.7); 1.8186 (1.5); 1.6255 (1.5); |
| | | 1.6131 (3.6); 1.6061 (3.7); 1.5926 (1.2); |
| | | 1.2647 (3.4); 1.2463 (7.7); 1.2277 (3.3); |
| | | 0.1459 (0.4); 0.0079 (3.4); −0.0002 (84.9); |
| | | −0.0085 (3.1); −0.1497 (0.4) |
| I-05 | 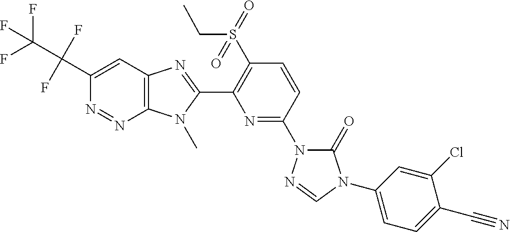 [SMILES: CCS(=O)(=O)c1ccc(-n2ncn(-c3ccc(C#N)c(Cl)c3)c2=O)nc1-c1nc2cc(C(F)(F)C(F)(F)F)nnc2n1C] | : ¹H-NMR(400.2 MHz, d₆-DMSO): δ = 9.0607 (7.1); 8.8688 (5.7); 8.7768 (3.3); 8.7546 (4.1); 8.5527 (3.8); 8.5305 (3.5); 8.3192 (0.4); 8.2703 (3.5); 8.2652 (3.6); 8.2446 (2.8); 8.2232 (3.5); 8.0678 (2.2); 8.0627 (2.0); 8.0464 (1.8); 8.0411 (1.7); 4.0682 (16.0); 3.8338 (0.9); 3.8153 (3.2); 3.7968 (3.2); 3.7783 (0.9); 3.3354 (122.5); 2.6764 (1.1); 2.6719 (1.4); 2.6674 (1.1); 2.5253 (4.6); 2.5117 (87.9); 2.5074 (172.1); 2.5028 (226.8); 2.4983 (172.0); 2.4940 (86.8); 2.3342 (1.0); 2.3298 (1.4); 2.3252 (1.0); 2.0765 (0.4); 1.2627 (3.6); 1.2442 (8.1); 1.2257 (3.5); −0.0002 (1.0) |
| I-06 | 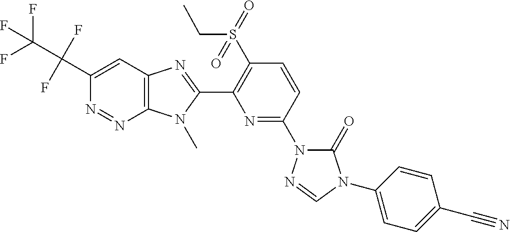 [SMILES: CCS(=O)(=O)c1ccc(-n2ncn(-c3ccc(C#N)cc3)c2=O)nc1-c1nc2cc(C(F)(F)C(F)(F)F)nnc2n1C] | : ¹H-NMR(400.2 MHz, d₆-DMSO): δ = 9.0145 (7.9); 8.8606 (6.6); 8.7703 (3.2); 8.7481 (4.0); 8.5668 (4.0); 8.5445 (3.6); 8.1055 (3.1); 8.1005 (1.3); 8.0885 (1.8); 8.0833 (5.9); 8.0337 (5.8); 8.0285 (1.7); 8.0165 (1.3); 8.0114 (3.2); 4.0726 (16.0); 3.8352 (0.9); 3.8167 (3.1); 3.7982 (3.2); 3.7797 (0.9); 3.3282 (74.2); 2.6763 (0.4); 2.6718 (0.6); 2.6672 (0.4); 2.5252 (1.8); 2.5117 (38.9); 2.5074 (77.6); 2.5028 (100.3); 2.4983 (72.0); 2.4938 (34.6); 2.3342 (0.4); 2.3297 (0.6); 2.3251 (0.4); 1.2646 (3.5); 1.2462 (8.1); 1.2277 (3.5); 0.0080 (1.1); −0.0002 (30.0); −0.0085 (1.1) |
| I-07 | 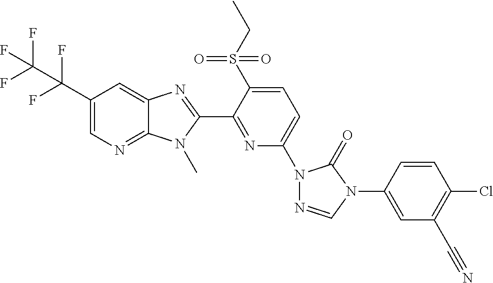 [SMILES: CCS(=O)(=O)c1ccc(-n2ncn(-c3ccc(Cl)c(C#N)c3)c2=O)nc1-c1nc2cc(C(F)(F)C(F)(F)F)cnc2n1C] | : ¹H-NMR(400.2 MHz, d₆-DMSO): δ = 8.9371 (7.2); 8.8587 (2.7); 8.8544 (2.9); 8.7361 (3.1); 8.7140 (3.8); 8.6813 (2.9); 8.6770 (2.8); 8.4906 (3.7); 8.4685 (3.3); 8.4070 (3.3); 8.4005 (3.5); 8.2015 (1.6); 8.1950 (1.5); 8.1793 (2.0); 8.1727 (2.0); 8.0068 (3.4); 7.9846 (2.8); 3.9073 (16.0); 3.8939 (3.5); 3.8754 (3.3); 3.8569 (1.0); 3.3282 (38.8); 2.6721 (0.4); 2.5075 (50.6); 2.5031 (65.2); 2.4987 (49.0); 2.3299 (0.4); 1.2668 (3.6); 1.2484 (7.9); 1.2299 (3.5); 0.0078 (0.6); −0.0002 (12.2); −0.0082 (0.6) |
| I-08 | 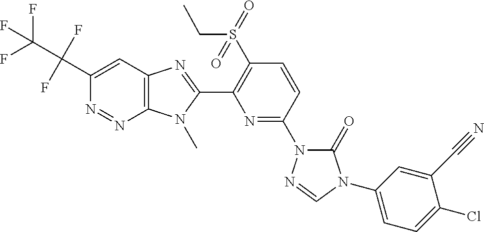 [SMILES: CCS(=O)(=O)c1ccc(-n2ncn(-c3ccc(Cl)c(C#N)c3)c2=O)nc1-c1nc2cc(C(F)(F)C(F)(F)F)nnc2n1C] | : ¹H-NMR(400.2 MHz, d₆-DMSO): δ = 8.9465 (6.8); 8.8620 (6.2); 8.7732 (3.0); 8.7510 (3.8); 8.5558 (3.6); 8.5336 (3.1); 8.4108 (3.3); 8.4042 (3.4); 8.3161 (0.7); 8.2600 (0.4); 8.2051 (1.8); 8.1984 (1.6); 8.1828 (2.1); 8.1764 (2.0); 8.0145 (3.4); 7.9923 (2.8); 4.0690 (16.0); 4.0378 (1.8); 3.8950 (0.8); 3.8323 (1.0); 3.8142 (3.2); 3.7956 (3.3); 3.7774 (1.0); 3.3265 (223.7); 2.6755 (2.2); 2.6713 (2.9); 2.6667 (2.1); 2.5708 (0.3); 2.5244 (8.7); 2.5066 (382.2); 2.5022 (478.0); 2.4978 (342.6); 2.3335 (2.1); 2.3291 (2.8); 2.3247 (2.0); 2.0747 (1.4); 1.2637 (3.7); 1.2453 (8.3); 1.2268 (3.6); −0.0001 (1.8) |
| I-09 | 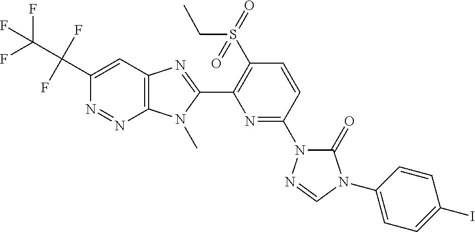 [SMILES: CCS(=O)(=O)c1ccc(-n2ncn(-c3ccc(I)cc3)c2=O)nc1-c1nc2cc(C(F)(F)C(F)(F)F)nnc2n1C] | : ¹H-NMR(400.2 MHz, d₆-DMSO): δ = 8.8734 (7.3); 8.8570 (6.5); 8.7536 (2.9); 8.7314 (3.8); 8.5679 (3.7); 8.5457 (3.1); 8.3163 (0.4); 7.9570 (4.4); 7.9353 (5.1); 7.8229 (0.8); 7.8148 (0.8); 7.8080 (0.7); 7.7957 (0.9); 7.7913 (0.8); 7.7829 (1.0); 7.5835 (5.1); 7.5617 (4.6); 7.4781 (1.0); 7.4653 (1.6); 7.4610 (1.6); 7.3847 (1.1); 7.3705 (0.4); 7.3445 (1.0); 4.0656 (16.0); 3.8281 (1.0); 3.8099 (3.4); 3.7914 (3.4); 3.7730 (1.1); 3.3261 (160.5); 2.6755 (1.2); 2.6713 (1.6); 2.6670 (1.2); 2.5067 (197.4); 2.5023 (252.7); 2.4981 (189.7); 2.3334 (1.2); 2.3291 (1.6); 2.3247 (1.2); 1.2609 (3.8); 1.2425 (8.3); 1.2240 (3.7); |
| | | 0.1458 (0.6); 0.0079 (6.6); −0.0001 |
| | | (122.0); −0.0078 (5.8); −0.1495 (0.6) |
| I-10 | 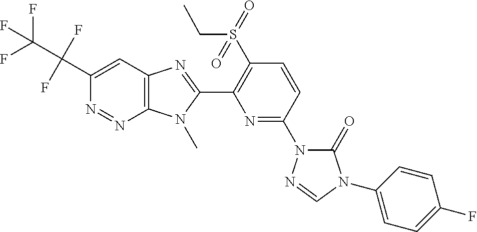 [SMILES: CCS(=O)(=O)c1ccc(-n2ncn(-c3ccc(F)cc3)c2=O)nc1-c1nc2cc(C(F)(F)C(F)(F)F)nnc2n1C] | : ¹H-NMR(400.2 MHz, d₆-DMSO): δ = 8.8583 (7.2); 8.8298 (8.2); 8.7562 (3.1); 8.7340 (4.0); 8.5770 (4.1); 8.5665 (0.3); 8.5548 (3.5); 7.7901 (2.3); 7.7847 (1.0); 7.7780 (2.5); 7.7728 (1.5); 7.7675 (2.8); 7.7608 (1.0); 7.7554 (2.6); 7.4663 (2.7); 7.4607 (0.9); 7.4442 (4.4); 7.4276 (0.8); 7.4221 (2.4); 5.7565 (0.6); 4.0703 (16.0); 4.0522 (0.6); 3.8310 (1.0); 3.8126 (3.4); 3.7941 (3.4); 3.7757 (1.0); 3.3264 (123.2); 2.6758 (0.7); 2.6713 (0.9); 2.6667 (0.7); 2.5246 (3.2); 2.5112 (57.6); 2.5069 (112.0); 2.5024 (145.3); 2.4978 (106.6); 2.4935 (52.6); 2.3336 (0.7); 2.3292 (0.9); 2.3246 (0.7); 2.0864 (0.4); 1.2630 (3.7); |
| | | 1.2445 (8.2); 1.2260 (3.7); 1.2110 (0.4); |
| | | 1.1402 (0.4); 0.0076 (1.6); −0.0002 (39.5); |
| | | −0.0083 (1.5) |
| I-11 | 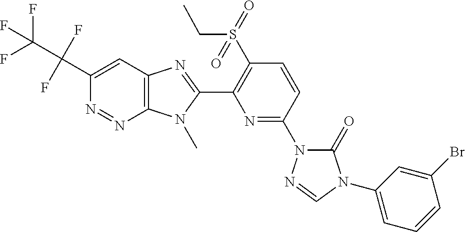 [SMILES: CCS(=O)(=O)c1ccc(-n2ncn(-c3cccc(Br)c3)c2=O)nc1-c1nc2cc(C(F)(F)C(F)(F)F)nnc2n1C] | : ¹H-NMR(400.2 MHz, d₆-DMSO): δ = 8.9183 (8.2); 8.8590 (7.5); 8.7612 (3.1); 8.7390 (4.0); 8.5699 (4.0); 8.5477 (3.5); 8.0378 (1.9); 8.0330 (3.5); 8.0282 (2.0); 7.8110 (1.5); 7.8081 (1.4); 7.7907 (1.7); 7.7880 (1.6); 7.6749 (1.3); 7.6727 (1.2); 7.6705 (1.1); 7.6547 (1.7); 7.6525 (1.8); 7.5672 (2.1); 7.5470 (3.2); 7.5267 (1.4); 7.4642 (0.5); 7.4598 (0.5); 7.4203 (0.3); 7.4151 (0.3); 7.3837 (0.4); 7.3433 (0.4); 7.3393 (0.4); 5.7569 (3.8); 4.0707 (16.0); 3.8310 (1.0); 3.8126 (3.4); 3.7941 (3.4); 3.7756 (1.0); 3.3262 (33.2); 2.6720 (0.4); 2.5252 (1.4); 2.5075 (52.6); 2.5030 (68.5); 2.4985 (51.1); 2.4943 (26.1); |
| | | 2.3343 (0.3); 2.3297 (0.4); 1.2642 (3.7); |
| | | 1.2458 (8.2); 1.2273 (3.6); 0.0078 (0.6); |
| | | −0.0002 (16.2); −0.0084 (0.7) |
| I-12 | 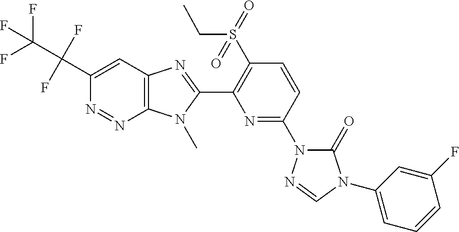 [SMILES: CCS(=O)(=O)c1ccc(-n2ncn(-c3cccc(F)c3)c2=O)nc1-c1nc2cc(C(F)(F)C(F)(F)F)nnc2n1C] | : ¹H-NMR(400.2 MHz, d₆-DMSO): δ = 8.9245 (8.0); 8.8592 (6.8); 8.7628 (3.2); 8.7406 (4.2); 8.5758 (4.1); 8.5536 (3.6); 7.7252 (1.0); 7.7220 (1.2); 7.7172 (0.9); 7.6963 (1.3); 7.6908 (0.9); 7.6673 (0.5); 7.6627 (0.4); 7.6552 (1.9); 7.6520 (2.2); 7.6470 (2.5); 7.6432 (2.6); 7.6247 (1.2); 7.6045 (0.4); 7.4643 (0.4); 7.4600 (0.4); 7.3842 (0.4); 7.3432 (0.8); 7.3356 (0.8); 7.3275 (0.8); 7.3208 (0.9); 7.3172 (0.8); 7.3147 (0.8); 7.3105 (0.6); 7.3023 (0.5); 7.2979 (0.6); 7.2919 (0.4); 4.0722 (16.0); 3.8329 (0.9); 3.8145 (3.3); 3.7960 (3.3); 3.7775 (1.0); 3.3276 (35.7); 2.6763 |
| | | (0.3); 2.6717 (0.5); 2.6674 (0.4); 2.5253 |
| | | (1.4); 2.5205 (2.2); 2.5118 (27.6); 2.5074 |
| | | (56.9); 2.5029 (76.3); 2.4983 (57.2); |
| | | 2.4939 (29.0); 2.3342 (0.3); 2.3296 (0.5); |
| | | 2.3250 (0.4); 2.0753 (4.4); 1.2646 (3.6); |
| | | 1.2462 (8.1); 1.2277 (3.5); 0.0079 (0.7); |
| | | −0.0002 (22.8); −0.0085 (0.9) |
| I-13 | 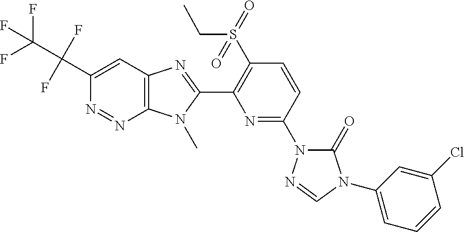 [SMILES: CCS(=O)(=O)c1ccc(-n2ncn(-c3cccc(Cl)c3)c2=O)nc1-c1nc2cc(C(F)(F)C(F)(F)F)nnc2n1C] | : ¹H-NMR(400.2 MHz, d₆-DMSO): δ = 8.9236 (6.9); 8.8600 (6.2); 8.7623 (3.0); 8.7401 (3.8); 8.5723 (3.7); 8.5501 (3.1); 8.3158 (0.5); 7.9147 (1.9); 7.9099 (3.4); 7.9051 (1.9); 7.7762 (1.4); 7.7582 (1.7); 7.7559 (1.7); 7.7534 (1.7); 7.6383 (1.5); 7.6181 (3.1); 7.5979 (1.8); 7.5463 (1.8); 7.5441 (1.9); 7.5417 (1.8); 7.5261 (1.2); 7.5239 (1.2); 7.5214 (1.2); 5.7560 (0.7); 4.0706 (16.0); 3.8310 (1.0); 3.8125 (3.4); 3.7940 (3.4); 3.7757 (1.0); 3.3236 (83.4); 2.6752 (1.5); 2.6709 (2.0); 2.6667 (1.5); 2.5063 (249.0); 2.5019 (312.7); 2.4976 (230.1); 2.3331 (1.5); 2.3289 (1.9); |
| | | 2.3244 (1.4); 1.2638 (3.9); 1.2453 (8.8); |
| | | 1.2347 (2.2); 1.2270 (4.1); 0.1462 (0.4); |
| | | 0.0076 (4.1); −0.0003 (88.5); −0.0084 |
| | | (3.7); −0.1496 (0.4) |
| I-14 | 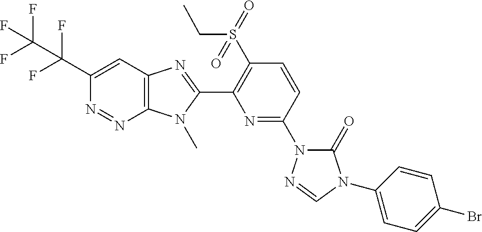 [SMILES: CCS(=O)(=O)c1ccc(-n2ncn(-c3ccc(Br)cc3)c2=O)nc1-c1nc2cc(C(F)(F)C(F)(F)F)nnc2n1C] | : ¹H-NMR(400.2 MHz, d₆-DMSO): δ = 8.8833 (8.2); 8.8574 (7.3); 8.7573 (3.2); 8.7351 (4.2); 8.5703 (4.1); 8.5481 (3.6); 7.8091 (3.6); 7.8038 (1.5); 7.7923 (1.8); 7.7868 (6.6); 7.7804 (1.0); 7.7421 (0.8); 7.7355 (6.3); 7.7301 (1.7); 7.7186 (1.3); 7.7132 (3.6); 7.4646 (0.4); 7.4600 (0.4); 7.3841 (0.4); 4.0692 (16.0); 3.8312 (0.9); 3.8127 (3.3); 3.7942 (3.3); 3.7758 (1.0); 3.3264 (42.3); 2.6762 (0.3); 2.6718 (0.5); 2.6675 (0.4); 2.5252 (1.2); 2.5204 (1.9); 2.5117 (27.6); 2.5073 (56.5); 2.5028 (75.2); 2.4982 (55.8); 2.4938 (27.9); 2.3342 (0.3); 2.3297 (0.5); 2.3249 (0.3); |
| | | 1.2628 (3.6); 1.2444 (8.2); 1.2259 (3.5); |
| | | 0.0080 (1.0); −0.0002 (32.8); −0.0084 (1.2) |
| I-15 | 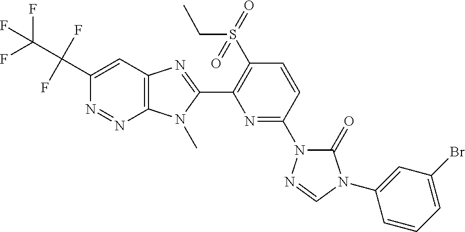 [SMILES: CCS(=O)(=O)c1ccc(-n2ncn(-c3cccc(Br)c3)c2=O)nc1-c1nc2cc(C(F)(F)C(F)(F)F)nnc2n1C] | : ¹H-NMR(400.2 MHz, d₆-DMSO): δ = 20.0119 (0.5); 19.2639 (0.7); 18.5128 (1.5); 16.0399 (0.4); 15.5098 (1.0); 12.5016 (0.5); 9.5488 (0.4); 9.5016 (7.0); 9.5001 (2.8); 9.4964 (0.8); 9.4936 (0.7); 9.4919 (0.7); 9.4787 (0.8); 9.4126 (0.4); 9.4083 (0.4); 9.1768 (0.4); 8.1011 (0.4); 8.0959 (0.5); 8.0943 (0.6); 8.0907 (0.4); 8.0591 (0.4); 8.0384 (0.5); 8.0323 (0.6); 8.0295 (0.4); 8.0237 (1.4); 8.0156 (1.2); 8.0149 (0.9); 8.0119 (1.4); 8.0111 (0.9); 8.0105 (1.6); 8.0097 (2.0); 8.0089 (2.4); 8.0068 (2.2); 8.0060 (3.2); 8.0053 (2.5); 8.0047 (2.0); 8.0038 (3.3); 8.0030 (3.2); |
| | | 8.0024 (4.7); 8.0017 (7.3); 8.0009 (16.0); |
| | | 8.0002 (15.9); 7.9727 (0.5); 7.9565 (0.5); |
| | | 7.9551 (0.5); 7.9533 (0.5); 7.8645 (0.4); |
| | | 7.7889 (0.5); 7.6530 (0.4); 7.0872 (0.4); |
| | | 6.4945 (2.1); 6.4914 (0.8); 6.4898 (0.8); |
| | | 6.4866 (0.9); 6.4808 (0.4); 6.4745 (0.7); |
| | | 3.4944 (3.3); 3.3295 (1.2); 2.5115 (0.8); |
| | | 2.5070 (1.1); 0.4998 (0.4); 0.4915 (1.3); |
| | | 0.0046 (0.7); −0.0400 (0.4); −1.7615 (0.4) |
| I-16 | 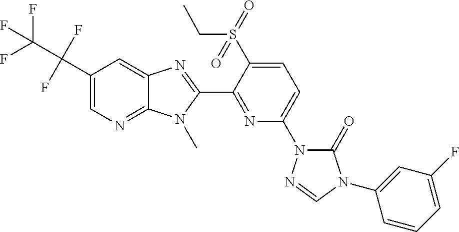 [SMILES: CCS(=O)(=O)c1ccc(-n2ncn(-c3cccc(F)c3)c2=O)nc1-c1nc2cc(C(F)(F)C(F)(F)F)cnc2n1C] | : ¹H-NMR(400.2 MHz, d₆-DMSO): δ = 8.9132 (7.8); 8.8599 (2.4); 8.8555 (2.5); 8.7265 (3.0); 8.7043 (3.8); 8.6855 (2.6); 8.6809 (2.5); 8.5129 (3.8); 8.4908 (3.4); 7.7229 (1.0); 7.7197 (1.1); 7.7153 (0.8); 7.6941 (1.2); 7.6886 (0.9); 7.6642 (0.5); 7.6598 (0.3); 7.6521 (1.9); 7.6490 (2.1); 7.6437 (2.5); 7.6402 (2.4); 7.6217 (1.1); 7.6015 (0.4); 7.3412 (0.5); 7.3340 (0.7); 7.3256 (0.7); 7.3189 (0.9); 7.3153 (0.8); 7.3129 (0.8); 7.3085 (0.5); 7.3005 (0.5); 7.2959 (0.6); 7.2899 (0.4); 3.9079 (16.0); 3.8911 (3.2); 3.8725 (3.2); 3.8540 (0.9); 3.3229 (65.9); 2.6753 (0.5); 2.6707 (0.7); 2.6662 (0.5); 2.5241 (2.1); 2.5106 |
| | | (41.6); 2.5063 (83.6); 2.5018 (109.4); |
| | | 2.4972 (79.2); 2.4928 (38.5); 2.3332 (0.5); |
| | | 2.3286 (0.6); 2.3241 (0.5); 2.0750 (1.0); |
| | | 1.2654 (3.4); 1.2470 (7.6); 1.2284 (3.3); |
| | | −0.0002 (0.4) |
| I-17 | 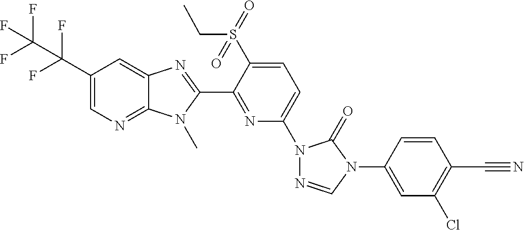 [SMILES: CCS(=O)(=O)c1ccc(-n2ncn(-c3ccc(C#N)c(Cl)c3)c2=O)nc1-c1nc2cc(C(F)(F)C(F)(F)F)cnc2n1C] | : ¹H-NMR(400.2 MHz, d₆-DMSO): δ = 9.0461 (7.4); 8.8626 (2.3); 8.8579 (2.4); 8.7394 (3.2); 8.7173 (4.0); 8.6871 (2.5); 8.6825 (2.4); 8.4878 (3.8); 8.4656 (3.5); 8.2664 (3.3); 8.2612 (3.4); 8.2383 (2.7); 8.2168 (3.4); 8.0630 (2.0); 8.0578 (1.8); 8.0416 (1.6); 8.0363 (1.6); 3.9903 (0.5); 3.9041 (16.0); 3.8904 (3.1); 3.8719 (3.0); 3.8527 (1.4); 3.8160 (0.4); 3.3217 (102.2); 2.6794 (0.5); 2.6751 (1.0); 2.6705 (1.4); 2.6659 (1.0); 2.6613 (0.5); 2.5240 (4.3); 2.5192 (6.5); 2.5106 (85.4); 2.5061 (173.2); 2.5015 (226.8); 2.4969 (162.8); 2.4924 (77.8); 2.3373 (0.4); 2.3330 (1.0); 2.3283 (1.4); 2.3237 (1.0); 2.3192 (0.4); |
| | | 2.0747 (0.9); 1.2645 (3.4); 1.2461 (7.9); |
| | | 1.2275 (3.4); 1.2053 (0.4); 0.0080 (2.3); |
| | | −0.0001 (72.0); −0.0085 (2.3) |
| I-18 | 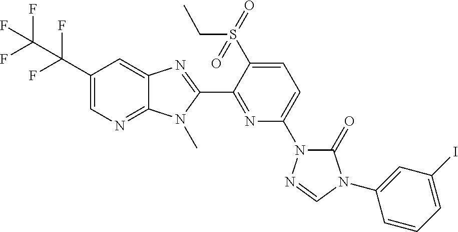 [SMILES: CCS(=O)(=O)c1ccc(-n2ncn(-c3cccc(I)c3)c2=O)nc1-c1nc2cc(C(F)(F)C(F)(F)F)cnc2n1C] | : ¹H-NMR(400.2 MHz, d₆-DMSO): δ = 8.8815 (8.0); 8.8592 (2.2); 8.8545 (2.3); 8.7205 (3.2); 8.6983 (4.1); 8.6846 (2.3); 8.6798 (2.2); 8.5061 (3.9); 8.4840 (3.6); 8.1556 (1.7); 8.1511 (2.8); 8.1465 (1.8); 7.8326 (1.1); 7.8303 (1.4); 7.8288 (1.3); 7.8264 (1.1); 7.8128 (1.1); 7.8106 (1.5); 7.8090 (1.4); 7.8067 (1.3); 7.8008 (1.1); 7.7987 (1.2); 7.7956 (1.1); 7.7935 (1.0); 7.7804 (1.2); 7.7782 (1.1); 7.7751 (1.3); 7.7730 (1.0); 7.3912 (1.7); 7.3711 (2.9); 7.3510 (1.4); 3.9032 (16.0); 3.8860 (2.9); 3.8675 (2.9); 3.8490 (0.8); 3.3220 (56.0); 2.6753 (0.5); 2.6706 (0.8); 2.6661 (0.6); 2.5242 (2.0); 2.5194 (3.0); 2.5107 |
| | | (44.0); 2.5062 (90.8); 2.5016 (120.3); |
| | | 2.4970 (86.5); 2.4925 (41.1); 2.3330 (0.5); |
| | | 2.3285 (0.7); 2.3239 (0.5); 2.0749 (6.6); |
| | | 1.2638 (3.2); 1.2453 (7.5); 1.2268 (3.1); |
| | | −0.0002 (3.9) |
| I-19 | 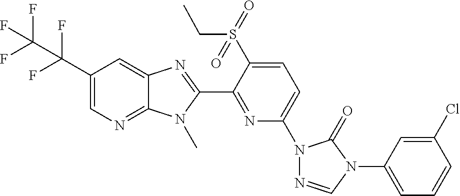 [SMILES: CCS(=O)(=O)c1ccc(-n2ncn(-c3cccc(Cl)c3)c2=O)nc1-c1nc2cc(C(F)(F)C(F)(F)F)cnc2n1C] | : ¹H-NMR(400.2 MHz, d₆-DMSO): δ = 8.9140 (6.9); 8.8583 (2.1); 8.8535 (2.3); 8.7259 (3.2); 8.7038 (4.0); 8.6818 (2.2); 8.6772 (2.1); 8.5100 (3.7); 8.4879 (3.4); 7.9126 (1.6); 7.9075 (3.2); 7.9025 (1.7); 7.7758 (0.9); 7.7734 (1.1); 7.7708 (0.9); 7.7683 (0.9); 7.7556 (1.2); 7.7532 (1.3); 7.7505 (1.3); 7.7480 (1.2); 7.6341 (1.4); 7.6138 (2.9); 7.5936 (1.7); 7.5425 (1.3); 7.5401 (1.5); 7.5375 (1.4); 7.5351 (1.3); 7.5223 (0.8); 7.5199 (0.9); 7.5173 (0.9); 7.5149 (0.8); 4.2749 (0.6); 3.9084 (16.0); 3.8913 (2.9); 3.8728 (2.9); 3.8544 |
| | | (0.8); 3.3256 (47.7); 2.6758 (0.5); 2.6712 |
| | | (0.8); 2.6666 (0.5); 2.5247 (2.2); 2.5200 |
| | | (3.1); 2.5113 (44.4); 2.5068 (91.8); 2.5023 |
| | | (121.0); 2.4977 (86.0); 2.4931 (40.6); |
| | | 2.3336 (0.5); 2.3291 (0.7); 2.3245 (0.5); |
| | | 2.0749 (1.9); 1.2663 (3.2); 1.2478 (7.6); |
| | | 1.2293 (3.3); 0.1458 (0.6); 0.0079 (4.2); |
| | | −0.0002 (139.5); −0.0086 (4.4); −0.0134 |
| | | (0.5); −0.1498 (0.6) |
| I-20 | 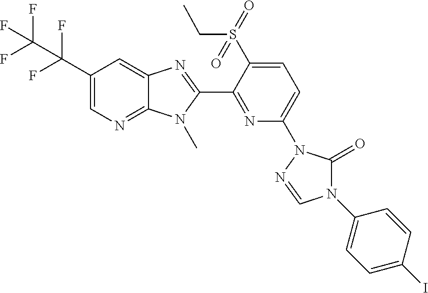 [SMILES: CCS(=O)(=O)c1ccc(-n2ncn(-c3ccc(I)cc3)c2=O)nc1-c1nc2cc(C(F)(F)C(F)(F)F)cnc2n1C] | : ¹H-NMR(400.2 MHz, d₆-DMSO): δ = 8.8598 (8.2); 8.8528 (2.4); 8.7172 (3.2); 8.6951 (4.0); 8.6828 (2.2); 8.6781 (2.2); 8.5052 (3.8); 8.4830 (3.5); 7.9604 (0.4); 7.9536 (4.2); 7.9485 (1.4); 7.9367 (1.4); 7.9316 (5.0); 7.9250 (0.6); 7.5865 (0.5); 7.5798 (4.8); 7.5746 (1.5); 7.5629 (1.4); 7.5577 (4.6); 7.5509 (0.5); 3.9015 (16.0); 3.8863 (2.9); 3.8678 (2.9); 3.8493 (0.9); 3.3449 (315.5); 2.6769 (0.4); 2.6723 (0.6); 2.6677 (0.5); 2.5258 (1.7); 2.5211 (2.6); 2.5124 (37.9); 2.5079 (78.8); 2.5033 (104.5); 2.4987 (74.6); 2.4941 (35.1); 2.3346 (0.4); 2.3301 (0.6); 2.3255 (0.4); 2.0751 (3.1); 1.2623 (3.2); 1.2439 (7.6); 1.2254 (3.2); −0.0002 (8.5) |
| I-21 | 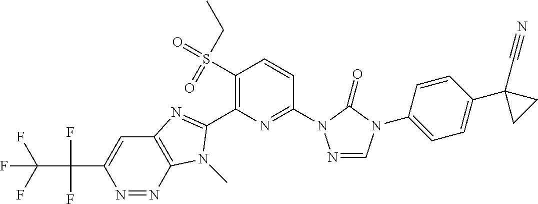 [SMILES: CCS(=O)(=O)c1ccc(-n2ncn(-c3ccc(C4(C#N)CC4)cc3)c2=O)nc1-c1nc2cc(C(F)(F)C(F)(F)F)nnc2n1C] | : ¹H-NMR(400.2 MHz, d₆-DMSO): δ = 8.8678 (8.5); 8.8576 (6.7); 8.7539 (3.1); 8.7317 (4.2); 8.5783 (4.1); 8.5561 (3.6); 8.3157 (0.5); 7.7734 (3.9); 7.7684 (1.4); 7.7568 (1.5); 7.7516 (5.0); 7.7450 (0.6); 7.5550 (0.6); 7.5485 (4.7); 7.5435 (1.5); 7.5318 (1.4); 7.5266 (4.0); 7.5200 (0.4); 7.3079 (0.4); 4.0673 (16.0); 4.0555 (0.6); 4.0377 (1.1); 4.0199 (1.1); 4.0020 (0.4); 3.8281 (0.9); 3.8097 (3.0); 3.7912 (3.1); 3.7728 (0.9); 3.5679 (3.8); 3.3243 (216.5); 2.6798 (0.5); 2.6753 (1.1); 2.6708 (1.5); 2.6662 (1.1); 2.6617 (0.6); 2.5243 (4.2); 2.5195 (6.6); 2.5109 (89.0); 2.5064 |
| | | (183.0); 2.5018 (242.5); 2.4972 (171.9); |
| | | 2.4927 (80.9); 2.3378 (0.5); 2.3332 (1.0); |
| | | 2.3286 (1.4); 2.3241 (1.0); 2.3196 (0.5); |
| | | 1.9888 (4.9); 1.8303 (1.2); 1.8173 (3.1); |
| | | 1.8104 (3.5); 1.7992 (1.4); 1.6001 (1.4); |
| | | 1.5875 (3.4); 1.5806 (3.5); 1.5668 (1.1); |
| | | 1.2616 (3.5); 1.2431 (8.3); 1.2246 (3.5); |
| | | 1.1927 (1.4); 1.1749 (2.8); 1.1571 (1.3); |
| | | 0.1459 (0.4); 0.0080 (3.0); −0.0002 (95.0); |
| | | −0.0085 (3.2); −0.1496 (0.4) |
| I-22 | 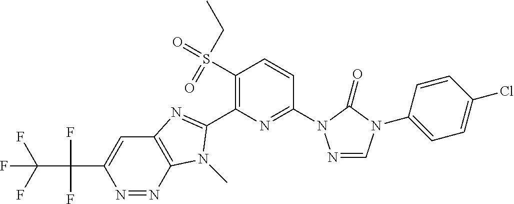 [SMILES: CCS(=O)(=O)c1ccc(-n2ncn(-c3ccc(Cl)cc3)c2=O)nc1-c1nc2cc(C(F)(F)C(F)(F)F)nnc2n1C] | : ¹H-NMR(400.2 MHz, d₆-DMSO): δ = 8.8777 (8.1); 8.8526 (7.2); 8.7573 (3.2); 8.7351 (4.1); 8.5704 (4.0); 8.5481 (3.5); 7.8067 (0.4); 7.7994 (4.1); 7.7940 (1.4); 7.7825 (1.7); 7.7771 (5.9); 7.7698 (0.6); 7.6825 (0.7); 7.6753 (5.9); 7.6699 (1.6); 7.6583 (1.4); 7.6531 (4.1); 7.6455 (0.4); 4.0698 (16.0); 3.8301 (1.0); 3.8116 (3.3); 3.7931 (3.4); 3.7747 (1.0); 3.3191 (81.4); 2.6755 (0.6); 2.6710 (0.8); 2.6665 (0.6); 2.5242 (2.7); 2.5108 (47.0); 2.5065 (90.4); 2.5019 (116.0); 2.4974 (83.9); 2.4930 (40.9); 2.3332 (0.5); 2.3288 (0.7); |
| | | 2.3243 (0.5); 2.0736 (0.4); 1.2635 (3.7); |
| | | 1.2450 (8.3); 1.2266 (3.6); 0.1460 (0.5); |
| | | 0.0160 (0.4); 0.0079 (4.8); −0.0002 |
| | | (121.8); −0.0085 (4.5); −0.1496 (0.5) |
| I-23 | 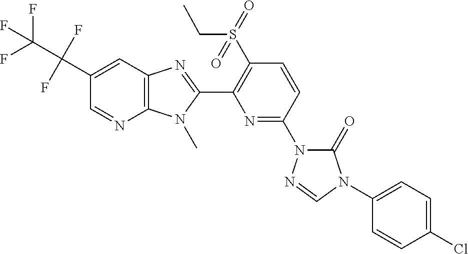 [SMILES: CCS(=O)(=O)c1ccc(-n2ncn(-c3ccc(Cl)cc3)c2=O)nc1-c1nc2cc(C(F)(F)C(F)(F)F)cnc2n1C] | : ¹H-NMR(400.0 MHz, d₆-DMSO): δ = 8.8686 (7.2); 8.8552 (2.6); 8.8508 (2.9); 8.7213 (3.1); 8.6991 (3.9); 8.6777 (2.7); 8.6733 (2.8); 8.5078 (3.7); 8.4857 (3.3); 7.8046 (0.4); 7.7972 (4.0); 7.7920 (1.5); 7.7803 (1.6); 7.7749 (5.7); 7.7675 (0.8); 7.6786 (0.7); 7.6713 (5.6); 7.6660 (1.8); 7.6542 (1.4); 7.6490 (4.0); 5.7555 (8.1); 3.9076 (16.0); 3.8910 (3.3); 3.8724 (3.2); 3.8541 (1.0); 3.3194 (25.1); 2.6714 (0.4); 2.5111 (20.9); 2.5068 (43.0); 2.5023 (59.5); 2.4979 (45.8); 2.3292 (0.4); 1.9891 (1.1); 1.2659 (3.4); 1.2474 (7.7); 1.2289 (3.7); 1.1931 (0.4); 1.1753 (0.7); 1.1575 (0.3); 0.0080 (2.6); −0.0001 (67.0); −0.0083 (2.8) |
| I-24 | 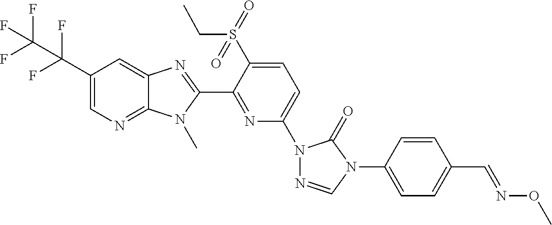 [SMILES: CCS(=O)(=O)c1ccc(-n2ncn(-c3ccc(/C=N/OC)cc3)c2=O)nc1-c1nc2cc(C(F)(F)C(F)(F)F)cnc2n1C] | : ¹H-NMR(600.1 MHz, d₆-DMSO): δ = 8.9314 (0.2); 8.9086 (5.9); 8.8556 (1.5); 8.8524 (1.5); 8.7178 (2.3); 8.7030 (2.8); 8.6778 (1.5); 8.6747 (1.4); 8.5157 (2.8); 8.5009 (2.7); 8.3138 (0.3); 8.3026 (3.7); 7.8288 (1.0); 7.8247 (0.5); 7.8180 (0.8); 7.8138 (4.8); 7.8050 (4.2); 7.8009 (0.6); 7.7939 (0.4); 7.7899 (0.9); 3.9698 (0.4); 3.9250 (16.0); 3.9093 (11.8); 3.8981 (0.6); 3.8858 (2.2); 3.8735 (2.2); 3.8611 (0.6); 3.3162 (151.8); 2.6194 (0.4); 2.6164 (0.8); 2.6133 (1.1); 2.6102 (0.8); 2.6073 (0.4); 2.5223 (2.4); 2.5193 (3.0); 2.5161 (2.7); 2.5074 (51.9); 2.5043 (115.3); |
| | | 2.5013 (161.2); 2.4982 (114.0); 2.4951 |
| | | (50.4); 2.3912 (0.3); 2.3882 (0.8); 2.3851 |
| | | (1.0); 2.3821 (0.7); 2.3790 (0.3); 1.2606 |
| | | (2.6); 1.2483 (5.9); 1.2360 (2.6); 0.0968 |
| | | (0.4); 0.0054 (3.3); −0.0001 (125.9); |
| | | −0.0057 (3.8); −0.1003 (0.5) |

### Use Examples

### Boophilus microplus—Injection Test

Solvent: dimethyl sulfoxide

An appropriate active ingredient formulation is produced by mixing 10 mg of active ingredient with 0.5 ml of solvent and diluting the concentrate with solvent to the desired concentration.

1 μl of the active ingredient solution is injected into the abdomen of 5 engorged adult female cattle ticks (Boophilus microplus). The ticks are transferred into dishes and kept in a climate-controlled room.

Efficacy is checked after 7 days by laying of fertile eggs. Eggs which are not visibly fertile are stored in a climate-controlled cabinet until the larvae hatch after about 42 days. Efficacy of 100% means that none of the ticks has laid any fertile eggs; 0% means that all the eggs are fertile.

In this test, for example, the following compounds from the preparation examples show an efficacy of 90% at an application rate of 20 μg/tick: I-23.

### Ctenocephalides felis—Oral Test

Solvent: dimethyl sulfoxide

An appropriate active ingredient formulation is produced by mixing 10 mg of active ingredient with 0.5 ml of dimethyl sulfoxide. Dilution with citrated cattle blood gives the desired concentration.

About 20 unfed adult cat fleas (Ctenocephalides felis) are placed into a chamber which is closed at the top and bottom with gauze. Onto the chamber is placed a metal cylinder with its bottom end closed with parafilm. The cylinder contains the blood/active ingredient formulation, which can be imbibed by the fleas through the parafilm membrane. The cylinder is heated to 37° C., while the flea chambers are kept at room temperature.

After 2 days, the kill rate in % is determined relative to the untreated control. 100% means that all of the fleas have been killed; 0% means that none of the fleas have been killed.

In this test, for example, the following compounds from the preparation examples show an efficacy of 100% at an application rate of 100 ppm: 1-22, 1-23.

### Lucilia cuprina Test

Solvent: dimethyl sulfoxide

An appropriate active ingredient formulation is produced by mixing 10 mg of active ingredient with 0.5 ml of dimethyl sulfoxide and diluting the concentrate with water to the desired concentration.

About 20 L1 larvae of the Australian sheep blowfly (Lucilia cuprina) are transferred into a test vessel containing minced horsemeat and the active ingredient formulation of the desired concentration.

After 2 days, the kill rate in % is determined relative to the untreated control. 100% means that all the larvae have been killed; 0% means that no larvae have been killed.

In this test, for example, the following compounds from the preparation examples show an efficacy of 100% at an application rate of 100 ppm: 1-22, 1-23.

### Musca domestica Test

Solvent: dimethyl sulfoxide

An appropriate active ingredient formulation is produced by mixing 10 mg of active ingredient with 0.5 ml of dimethyl sulfoxide and diluting the concentrate with water to the desired concentration.

Vessels containing a sponge treated with sugar solution and the desired concentration of active compound formulation are populated with 10 adult houseflies (Musca domestica).

After 2 days, the kill rate in % is determined relative to the untreated control. 100% means that all of the flies have been killed; 0% means that none of the flies have been killed.

In this test, for example, the following compounds from the preparation examples show an efficacy of 100% at an application rate of 100 ppm: 1-22, 1-23.

### Diabrotica balteata—Spray Test

Solvent: 78 parts by weight of acetone
1.5 parts by weight of dimethylformamide

Emulsifier: alkylaryl polyglycol ether

An appropriate active ingredient formulation is produced by dissolving 1 part by weight of active ingredient with the stated parts by weight of solvent and making the solution up to the desired concentration with water containing an emulsifier concentration of 1000 ppm. Further test concentrations are produced by diluting the formulation with emulsifier-containing water.

Pre-swollen wheat grains (Triticum aestivum) are incubated in a multiwell plate filled with agar and a little water for one day (5 seed grains per cavity). The germinated wheat grains are sprayed with an active ingredient formulation of the desired concentration. Subsequently, each cavity is infected with 10-20 beetle larvae of Diabrotica balteata.

After 7 days, efficacy in % is determined. 100% means that all wheat plants have grown as in the untreated, uninfected control; 0% means that no wheat plant has grown.

In this test, for example, the following compounds from the preparation examples show an efficacy of 100% at an application rate of 125 g/ha (=40 μg/cavity): I-20, I-21.

In this test, for example, the following compounds from the preparation examples show an efficacy of 100% at an application rate of 100 g/ha (=32 μg/cavity): I-01, I-02, I-03, I-04, I-05, I-06, I-07, I-08, I-09, I-10, I-11, I-12, I-13, I-14, I-15, I-16, I-17, 1-18, I-19, I-24.

### Meloidogyne incognita Test

Solvent: 125.0 parts by weight of acetone

An appropriate active ingredient formulation is produced by mixing 1 part by weight of active ingredient with the stated amount of solvent and diluting the concentrate to the desired concentration with water.

Vessels are filled with sand, active ingredient solution, an egg/larvae suspension of the southern root-knot nematode (Meloidogyne incognita) and lettuce seeds. The lettuce seeds germinate and the plants develop. The galls develop on the roots.

After 14 days, the nematicidal efficacy in % is determined by the formation of galls. 100% means that no galls were found; 0% means that the number of galls on the treated plants corresponds to the untreated control.

In this test, for example, the following compounds from the preparation examples show an efficacy of 100% at an application rate of 20 ppm: I-02, I-05, I-07, I-08, I-10, I-11, I-12, I-14, I-15, I-16.

In this test, for example, the following compounds from the preparation examples show an efficacy of 90% at an application rate of 20 ppm: I-09, I-17, I-20, I-21.

### Myzus persicae—Oral Test

Solvent: 100 parts by weight of acetone

An appropriate active ingredient formulation is produced by dissolving 1 part by weight of active ingredient with the specified parts by weight of solvent and making the solution up to the desired concentration with water.

50 μl of the active ingredient formulation is transferred into microtitre plates and made up to a final volume of 200 μl with 150 μl of IPL41 insect medium (33%+15% sugar). Subsequently, the plates are sealed with parafilm, which a mixed population of green peach aphids (Myzus persicae) within a second microtitre plate is able to puncture and imbibe the solution.

After 5 days, efficacy in % is determined. 100% means that all the aphids have been killed; 0% means that no aphids have been killed.

In this test, for example, the following compounds from the preparation examples show an efficacy of 100% at an application rate of 4 ppm: I-21, I-22, I-23.

In this test, for example, the following compounds from the preparation examples show an efficacy of 90% at an application rate of 4 ppm: I-17.

### Nezara viridula—Spray Test

Solvent: 78.0 parts by weight of acetone
1.5 parts by weight of dimethylformamide

Emulsifier: alkylaryl polyglycol ether

An appropriate active ingredient formulation is produced by dissolving 1 part by weight of active ingredient with the stated parts by weight of solvent and making the solution up to the desired concentration with water containing an emulsifier concentration of 1000 ppm. Further test concentrations are produced by diluting the formulation with emulsifier-containing water.

Barley plants (Hordeum vulgare) are sprayed with an active ingredient formulation of the desired concentration and are infected with larvae of the Southern green shield bug (Nezara viridula).

After 4 days, efficacy in % is determined. 100% means that all of the shield bugs have been killed; 0% means that none of the shield bugs have been killed.

In this test, for example, the following compounds of the Preparation Examples show an efficacy of 100% at an application rate of 500 g/ha: I-02, I-04, I-06, I-07.

### Phaedon cochleariae—Spray Test

Solvent: 78.0 parts by weight of acetone
1.5 parts by weight of dimethylformamide

Emulsifier: alkylaryl polyglycol ether

An appropriate active ingredient formulation is produced by dissolving 1 part by weight of active ingredient with the stated parts by weight of solvent and making the solution up to the desired concentration with water containing an emulsifier concentration of 1000 ppm. Further test concentrations are produced by diluting the formulation with emulsifier-containing water.

Discs of Chinese cabbage leaves (Brassica pekinensis) are sprayed with an active ingredient formulation of the desired concentration and, after drying, populated with larvae of the mustard beetle (Phaedon cochleariae).

After 7 days, efficacy in % is determined. 100% means that all the beetle larvae have been killed; 0% means that no beetle larvae have been killed.

In this test, for example, the following compounds of the Preparation Examples show an efficacy of 100% at an application rate of 100 g/ha: 1-22, 1-23.

### Spodoptera Frusiperda—Spray Test

Solvent: 78.0 parts by weight of acetone
1.5 parts by weight of dimethylformamide

Emulsifier: alkylaryl polyglycol ether

An appropriate active ingredient formulation is produced by dissolving 1 part by weight of active ingredient with the stated parts by weight of solvent and making the solution up to the desired concentration with water containing an emulsifier concentration of 1000 ppm. Further test concentrations are produced by diluting the formulation with emulsifier-containing water.

Leaf discs of maize (Zea mays) are sprayed with an active ingredient formulation of the desired concentration and, after drying, populated with caterpillars of the fall armyworm (Spodoptera frugiperda).

After 7 days, efficacy in % is determined. 100% means that all the caterpillars have been killed; 0% means that no caterpillar has been killed.

In this test, for example, the following compounds of the Preparation Examples show an efficacy of 100% at an application rate of 100 g/ha: I-01, I-04, I-05, I-06, I-07, I-08, I-09, I-10, I-11, I-12, I-13, I-14, I-15, I-16, I-17, I-18, I-19, I-20, I-21, I-22, I-23, I-24.

### Tetranychus urticae—Spray Test, OP-Resistant

Solvent: 78.0 parts by weight of acetone
1.5 parts by weight of dimethylformamide

Emulsifier: alkylaryl polyglycol ether

An appropriate active ingredient formulation is produced by dissolving 1 part by weight of active ingredient with the stated parts by weight of solvent and making the solution up to the desired concentration with water containing an emulsifier concentration of 1000 ppm. Further test concentrations are produced by diluting the formulation with emulsifier-containing water.

Discs of bean leaves (Phaseolus vulgaris) infested with all stages of the greenhouse red spider mite (Tetranychus urticae) are sprayed with an active ingredient formulation of the desired concentration.

After 6 days, efficacy in % is determined. 100% means that all the spider mites have been killed; 0% means that no spider mites have been killed.

In this test, for example, the following compounds from the preparation examples show an efficacy of 90% at an application rate of 500 g/ha: I-03.

### Diabrotica balteata—Drench Test

Solvent: 7 parts by weight of dimethylformamide

Emulsifier: 2 parts by weight of alkylaryl polyglycol ether

An appropriate active ingredient formulation is produced by mixing 1 part by weight of active ingredient with the specified amounts of solvent and emulsifier, and diluting the concentrate to the desired concentration with water, it being necessary to take account of the volume of soil which is drenched. It should be ensured that a concentration of 40 ppm of emulsifier in the soil is not exceeded. Further test concentrations are produced by diluting with water.

5 maize cobs (Zea mays) in each case are sown into pots filled with earth, and the next day the pots are watered with the active ingredient preparation of the desired concentration. After one day, about 25 L2 larvae of the banded cucumber beetle (Diabrotica balteata) are added.

After 8 days, efficacy in % is determined. 100% means that all 5 plants have germinated and grown; 0% means that none of the plants has emerged.

In this test, for example, the following compounds from the preparation examples show an efficacy of 100% at an application rate of 20 ppm: 1-22, 1-23.

### Myzus persicae—Spray Test

Solvent: 14 parts by weight of dimethylformamide

Emulsifier: alkylaryl polyglycol ether

An appropriate active ingredient formulation is produced by dissolving 1 part by weight of active ingredient with the stated parts by weight of solvent and making the solution up to the desired concentration with water containing an emulsifier concentration of 1000 ppm. Further test concentrations are produced by diluting the formulation with emulsifier-containing water. If the addition of ammonium salts or/and penetrants is required, these are each added in a concentration of 1000 ppm to the formulation solution.

Bell pepper plants (Capsicum annuum) severely infested with the green peach aphid (Myzus persicae) are treated by spraying with the active ingredient formulation in the desired concentration.

After 6 days, the kill rate in % is determined. 100% means that all of the aphids have been killed; 0% means that none of the aphids have been killed.

In this test, for example, the following compounds from the preparation examples show an efficacy of 95% at an application rate of 20 ppm: I-06.

In this test, for example, the following compounds from the preparation examples show an efficacy of 98% at an application rate of 20 ppm: I-12.

### Nezara viridula—Spray Test

Solvent: 52.5 parts by weight of acetone
7 parts by weight of dimethylformamide

Emulsifier: alkylaryl polyglycol ether

An appropriate active ingredient formulation is produced by dissolving 1 part by weight of active ingredient with the stated parts by weight of solvent and making the solution up to the desired concentration with water containing an emulsifier concentration of 1000 ppm. Further test concentrations are produced by diluting the formulation with emulsifier-containing water. If the addition of ammonium salts or/and penetrants is required, these are each added in a concentration of 1000 ppm to the formulation solution.

Barley plants (Hordeum vulgare) infected with larvae of the Southern green shield bug (Nezara viridula) are sprayed with an active ingredient formulation of the desired concentration.

After 4 days, efficacy in % is determined. 100% means that all of the shield bugs have been killed; 0% means that none of the shield bugs have been killed.

In this test, for example, the following compounds from the preparation examples show an efficacy of 100% at an application rate of 100 g ai/ha: I-12, I-13, I-15, I-20, I-23.

In this test, for example, the following compounds from the preparation examples show an efficacy of 90% at an application rate of 100 g ai/ha: I-11, I-18, I-22.

### Spodoptera Frustiperda—Spray Test

Solvent: 14 parts by weight of dimethylformamide

Emulsifier: alkylaryl polyglycol ether

An appropriate active ingredient formulation is produced by dissolving 1 part by weight of active ingredient with the stated parts by weight of solvent and making the solution up to the desired concentration with water containing an emulsifier concentration of 1000 ppm. Further test concentrations are produced by diluting the formulation with emulsifier-containing water. If the addition of ammonium salts or/and penetrants is required, these are each added in a concentration of 1000 ppm to the formulation solution.

Cotton leaves (Gossypium hirsutum) are sprayed with an active ingredient formulation of the desired concentration and populated with caterpillars of the armyworm (Spodoptera frugiperda).

After 7 days, the kill in % is determined. 100% means that all the caterpillars have been killed; 0% means that none of the caterpillars have been killed.

In this test, for example, the following compounds from the preparation examples show an efficacy of 100% at an application rate of 4 ppm: I-02.

### Comparative Experiments

### Plutella xylostella Spray Test (PLUTMA)

Solvent: 14 parts by weight of dimethylformamide

Emulsifier: alkylaryl polyglycol ether

An appropriate active ingredient formulation is produced by dissolving 1 part by weight of active ingredient with the stated parts by weight of solvent and making the solution up to the desired concentration with water containing an emulsifier concentration of 1000 ppm. Further test concentrations are produced by diluting the formulation with emulsifier-containing water. If the addition of ammonium salts or/and penetrants is required, these are each added in a concentration of 1000 ppm to the formulation solution.

Cabbage leaves (Brassica oleracea) are sprayed with an active ingredient formulation of the desired concentration and infected with larvae of the diamondback moth (Plutella xylostella).

After 7 days, the kill in % is determined. 100% means that all caterpillars have been killed; 0% means that none of the caterpillars have been killed.

In this test, for example, the following compounds from the preparation examples show superior efficacy to the prior art: see table.

### Heliothis Armigera Spray Test (HELIAR)

Solvent: 14 parts by weight of dimethylformamide

Emulsifier: alkylaryl polyglycol ether

An appropriate active ingredient formulation is produced by dissolving 1 part by weight of active ingredient with the stated parts by weight of solvent and making the solution up to the desired concentration with water containing an emulsifier concentration of 1000 ppm. Further test concentrations are produced by diluting the formulation with emulsifier-containing water. If the addition of ammonium salts or/and penetrants is required, these are each added in a concentration of 1000 ppm to the formulation solution.

Cotton plants (Gossypium hirsutum) are sprayed with an active ingredient formulation of the desired concentration and, after drying, populated with caterpillars of the cotton bollworm (Heliothis armigera).

After 7 days, the kill in % is determined. 100% means that all caterpillars have been killed; 0% means that none of the caterpillars have been killed.

In this test, for example, the following compound of the Preparation Examples shows superior efficacy to the prior art: see table

### Spodoptera frugtiperda—Spray Test (SPODFR)

Solvent: 14 parts by weight of dimethylformamide

Emulsifier: alkylaryl polyglycol ether

An appropriate active ingredient formulation is produced by dissolving 1 part by weight of active ingredient with the stated parts by weight of solvent and making the solution up to the desired concentration with water containing an emulsifier concentration of 1000 ppm. Further test concentrations are produced by diluting the formulation with emulsifier-containing water. If the addition of ammonium salts or/and penetrants is required, these are each added in a concentration of 1000 ppm to the formulation solution.

Cotton leaves (Gossypium hirsutum) are sprayed with an active ingredient formulation of the desired concentration and populated with caterpillars of the armyworm (Spodoptera frugiperda).

After 7 days, the kill in % is determined. 100% means that all caterpillars have been killed; 0% means that none of the caterpillars have been killed.

In this test, for example, the following compounds from the preparation examples show superior efficacy to the prior art: see table.

| | | | | % |
|---|---|---|---|---|
| Substance | Structure | Object | Concentration | Efficacy |
| Ex. No. I-33 known from WO 2017/055185 | 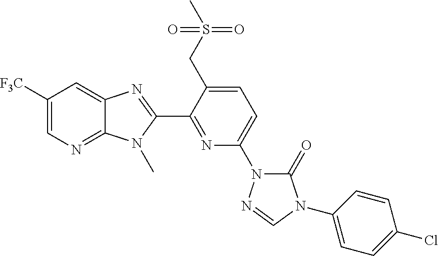 [SMILES: Cn1c(-c2nc(-n3ncn(-c4ccc(Cl)cc4)c3=O)ccc2CS(C)(=O)=O)nc2cc(C(F)(F)F)cnc21] | PLUTMA SPODFR | 0.16 ppm 0.8 ppm | 15 0 |
| Ex. No. I-23 inventive | 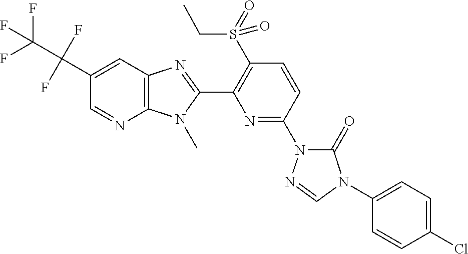 [SMILES: CCS(=O)(=O)c1ccc(-n2ncn(-c3ccc(Cl)cc3)c2=O)nc1-c1nc2cc(C(F)(F)C(F)(F)F)cnc2n1C] | PLUTMA SPODFR | 0.16 ppm 0.8 ppm | 90 100 |
| Ex. No. I-16 known from WO 2017/055185 | 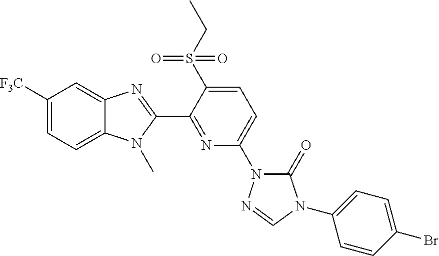 [SMILES: CCS(=O)(=O)c1ccc(-n2ncn(-c3ccc(Br)cc3)c2=O)nc1-c1nc2cc(C(F)(F)F)ccc2n1C] | PLUTMA SPODFR HELIAR HELIAR | 0.8 ppm 0.8 ppm 4 ppm 0.8 ppm | 85 10 60 20 |
| Ex. No. I-14 inventive | 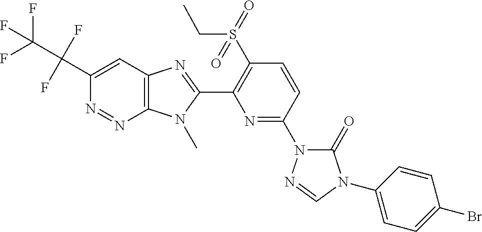 [SMILES: CCS(=O)(=O)c1ccc(-n2ncn(-c3ccc(Br)cc3)c2=O)nc1-c1nc2cc(C(F)(F)C(F)(F)F)nnc2n1C] | PLUTMA SPODFR HELIAR HELIAR | 0.8 ppm 0.8 ppm 4 ppm 0.8 ppm | 100 100 100 100 |

1. A compound of formula (I) 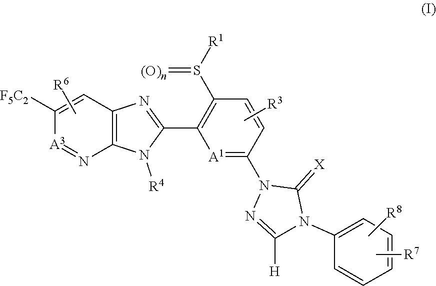
in which
A¹ is nitrogen, =N⁺(O⁻)— or =C(H)—,
A³ is nitrogen, =N⁺(O⁻)— or =C(H)—,
X is oxygen or sulfur,
R¹ is (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₈)cycloalkyl, halo(C₃-C₅)cycloalkyl, (C₃-C₈)cycloalkyl-(C₁-C₆)alkyl, (C₃-C₈)cycloalkyl-(C₁-C₆)haloalkyl, (C₁-C₆)alkyl-(C₃-C₈)cycloalkyl, (C₁-C₆)haloalkyl-(C₃-C₈)cycloalkyl, (C₁-C₆)cyanoalkyl, (C₁-C₆)alkoxy-(C₁-C₆)alkyl, (C₁-C₆)haloalkoxy-(C₁-C₆)alkyl, (C₁-C₆)alkylthio-(C₁-C₆)alkyl, (C₁-C₆)alkylsulfinyl-(C₁-C₆)alkyl or (C₁-C₆)alkylsulfonyl-(C₁-C₆)alkyl,
R³ is hydrogen, cyano, halogen, nitro, hydroxyl, amino, SCN, tri-(C₁-C₆)alkylsilyl, (C₃-C₈)cycloalkyl, (C₃-C₈)cycloalkyl-(C₃-C₈)cycloalkyl, (C₁-C₆)alkyl-(C₃-C₈)cycloalkyl, halo(C₃-C₅)cycloalkyl, cyano(C₃-C₈)cycloalkyl, (C₁-C₆)alkyl, (C₁-C₆)haloalkyl, (C₁-C₆)cyanoalkyl, (C₁-C₆)hydroxyalkyl, (C₁-C₆)alkoxy-(C₁-C₆)alkyl, (C₂-C₆)alkenyl, (C₂-C₆)haloalkenyl, (C₂-C₆)cyanoalkenyl, (C₂-C₆)alkynyl, (C₂-C₆)haloalkynyl, (C₂-C₆)cyanoalkynyl, (C₁-C₆)alkoxy, (C₁-C₆)haloalkoxy, (C₁-C₆)cyanoalkoxy, (C₁-C₆)alkylhydroxyimino, (C₁-C₆)alkoxyimino, (C₁-C₆)alkyl-(C₁-C₆)alkoxyimino, (C₁-C₆)haloalkyl-(C₁-C₆)alkoxyimino, (C₁-C₆)alkylthio, (C₁-C₆)haloalkylthio, (C₁-C₆)alkylsulfinyl, (C₁-C₆)haloalkylsulfinyl, (C₁-C₆)alkylsulfonyl, (C₁-C₆)haloalkylsulfonyl, (C₁-C₆)alkylcarbonyl, (C₁-C₆)haloalkylcarbonyl, (C₁-C₆)alkoxycarbonyl, (C₁-C₆)haloalkoxycarbonyl, aminocarbonyl, (C₁-C₆)alkylaminocarbonyl, di(C₁-C₆)alkylaminocarbonyl, (C₁-C₆)alkylsulfonylamino, (C₁-C₆)alkylamino, di(C₁-C₆)alkylamino, aminosulfonyl, (C₁-C₆)alkylaminosulfonyl, di(C₁-C₆)alkylaminosulfonyl, (C₁-C₆)alkylsulfoximino, (C₃-C₈)cycloalkylamino or NHCO—(C₁-C₆)alkyl ((C₁-C₆)alkylcarbonylamino),
R⁴ is hydrogen, (C₁-C₄)alkyl, (C₁-C₄)haloalkyl, (C₁-C₄)cyanoalkyl, (C₁-C₄)alkoxy-(C₁-C₄)alkyl, (C₂-C₄)alkenyl, (C₂-C₄)haloalkenyl, (C₂-C₄)cyanoalkenyl, (C₂-C₄)alkynyl, (C₂-C₄)haloalkynyl, (C₂-C₄)cyanoalkynyl, (C₁-C₄)alkoxy, (C₁-C₄)haloalkoxy, (C₁-C₄)alkylthio, (C₁-C₄)haloalkylthio, (C₁-C₄)alkylsulfinyl, (C₁-C₄)haloalkylsulfinyl, (C₁-C₄)alkylsulfonyl or (C₁-C₄)haloalkylsulfonyl,
R⁶ is hydrogen, cyano, halogen, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₈)cycloalkyl, (C₃-C₈)cycloalkyl-(C₃-C₈)cycloalkyl, (C₁-C₆)alkyl-(C₃-C₈)cycloalkyl, (C₁-C₆)alkoxy, (C₁-C₆)haloalkoxy, (C₁-C₆)alkoxyimino, (C₁-C₆)alkylthio, (C₁-C₆)haloalkylthio, (C₁-C₆)alkylsulfinyl, (C₁-C₆)haloalkylsulfinyl, (C₁-C₆)alkylsulfonyl, (C₁-C₆)haloalkylsulfonyl, (C₁-C₆)alkylsulfonyloxy, (C₁-C₆)alkylcarbonyl, (C₁-C₆)haloalkylcarbonyl, aminocarbonyl, (C₁-C₆)alkylaminocarbonyl, di(C₁-C₆)alkylaminocarbonyl, (C₁-C₆)alkylsulfonylamino, aminosulfonyl, (C₁-C₆)alkylaminosulfonyl or di(C₁-C₆)alkylaminosulfonyl,
R⁷, R⁸ are independently hydrogen, cyano, halogen, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₈)cycloalkyl, halo(C₃-C₅)cycloalkyl, cyano(C₃-C₈)cycloalkyl, (C₃-C₈)cycloalkyl-(C₃-C₈)cycloalkyl, (C₁-C₆)alkyl-(C₃-C₈)cycloalkyl, (C₁-C₆)alkoxy, (C₁-C₆)haloalkoxy, (C₁-C₆)alkoxyimino, (C₁-C₆)alkylthio, (C₁-C₆)haloalkylthio, (C₁-C₆)alkylsulfinyl, (C₁-C₆)haloalkylsulfinyl, (C₁-C₆)alkylsulfonyl, (C₁—C₆)haloalkylsulfonyl, (C₁-C₆)alkylcarbonyl, (C₁-C₆)haloalkylcarbonyl, (C₁-C₆)alkoxycarbonyl, aminocarbonyl, (C₁-C₆)alkylaminocarbonyl, di(C₁-C₆)alkylaminocarbonyl, (C₁-C₆)alkylsulfonylamino, aminosulfonyl, (C₁-C₆)alkylaminosulfonyl or di(C₁-C₆)alkylaminosulfonyl,
n is 0, 1 or 2.

2. The compound of formula (I) according to claim 1, in which
A¹ is nitrogen, =N⁺(O⁻)— or =C(H)—,
A³ is nitrogen, =N⁺(O⁻)— or =C(H)—,
X is oxygen or sulfur,
R¹ is (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₈)cycloalkyl, halo(C₃-C₅)cycloalkyl, (C₃-C₈)cycloalkyl-(C₁-C₆)alkyl, (C₃-C₈)cycloalkyl-(C₁-C₆)haloalkyl, (C₁-C₆)alkyl-(C₃-C₈)cycloalkyl, (C₁-C₆)haloalkyl-(C₃-C₈)cycloalkyl, (C₁-C₆)cyanoalkyl, (C₁-C₆)alkoxy-(C₁-C₆)alkyl or (C₁-C₆)haloalkoxy-(C₁-C₆)alkyl,
R³ is hydrogen, cyano, halogen, (C₃-C₈)cycloalkyl, (C₁-C₆)alkyl-(C₃-C₈)cycloalkyl, halo(C₃-C₅)cycloalkyl, cyano(C₃-C₈)cycloalkyl, (C₁-C₆)alkyl, (C₁-C₆)haloalkyl, (C₁-C₆)cyanoalkyl, (C₁-C₆)alkoxy-(C₁-C₆)alkyl, (C₂-C₆)alkenyl, (C₂-C₆)haloalkenyl, (C₂-C₆)cyanoalkenyl, (C₂-C₆)alkynyl, (C₂-C₆)haloalkynyl, (C₂-C₆)cyanoalkynyl, (C₁-C₆)alkoxy, (C₁-C₆)haloalkoxy, (C₁-C₆)cyanoalkoxy, (C₁-C₆)alkylhydroxyimino, (C₁-C₆)alkoxyimino, (C₁-C₆)alkyl-(C₁-C₆)alkoxyimino, (C₁-C₆)haloalkyl-(C₁-C₆)alkoxyimino, (C₁-C₆)alkylthio, (C₁-C₆)haloalkylthio, (C₁-C₆)alkylsulfinyl, (C₁-C₆)haloalkylsulfinyl, (C₁-C₆)alkylsulfonyl, (C₁-C₆)haloalkylsulfonyl, aminocarbonyl, (C₁-C₆)alkylaminocarbonyl, di(C₁-C₆)alkylaminocarbonyl, (C₁-C₆)alkylsulfonylamino, aminosulfonyl, (C₁-C₆)alkylaminosulfonyl, di(C₁-C₆)alkylaminosulfonyl, (C₁-C₆)alkylsulfoximino or NHCO—(C₁-C₆)alkyl ((C₁-C₆)alkylcarbonylamino),
R⁴ is hydrogen, (C₁-C₄)alkyl, (C₁-C₄)alkoxy-(C₁-C₄)alkyl, (C₁-C₄)haloalkyl, (C₂-C₄)alkenyl, (C₂-C₄)haloalkenyl, (C₂-C₄)alkynyl, (C₂-C₄)haloalkynyl, (C₁-C₄)alkoxy, (C₁-C₄)haloalkoxy, (C₁-C₄)alkylthio, (C₁-C₄)haloalkylthio, (C₁-C₄)alkylsulfinyl, (C₁-C₄)haloalkylsulfinyl, (C₁-C₄)alkylsulfonyl or (C₁-C₄)haloalkylsulfonyl,
R⁶ is hydrogen, cyano, halogen, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₈)cycloalkyl, (C₁-C₆)alkyl-(C₃-C₈)cycloalkyl, (C₁-C₆)alkoxy, (C₁-C₆)haloalkoxy, (C₁-C₆)alkoxyimino, (C₁-C₆)alkylthio, (C₁-C₆)haloalkylthio, (C₁-C₆)alkylsulfinyl, (C₁-C₆)haloalkylsulfinyl, (C₁-C₆)alkylsulfonyl, (C₁-C₆)haloalkylsulfonyl, (C₁-C₆)alkylcarbonyl or (C₁-C₆)haloalkylcarbonyl,
R⁷, R⁸ are independently hydrogen, cyano, halogen, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₈)cycloalkyl, halo(C₃-C₅)cycloalkyl, cyano(C₃-C₈)cycloalkyl, (C₃-C₈)cycloalkyl-(C₃-C₈)cycloalkyl, (C₁-C₆)alkyl-(C₃-C₈)cycloalkyl, (C₁-C₆)alkoxy, (C₁-C₆)haloalkoxy, (C₁-C₆)alkoxycarbonyl, (C₁-C₆)alkoxyimino, (C₁-C₆)alkylthio, (C₁-C₆)haloalkylthio, (C₁-C₆)alkylsulfinyl, (C₁-C₆)haloalkylsulfinyl, (C₁-C₆)alkylsulfonyl or (C₁-C₆)haloalkylsulfonyl,
n is 0, 1 or 2.

3. The compound of formula (I) according to claim 1, in which
A¹ is nitrogen, =N⁺(O⁻)— or =C(H)—,
A³ is nitrogen, =N⁺(O⁻)— or =C(H)—,
X is oxygen or sulfur,
R¹ is (C₁-C₆)alkyl, (C₁-C₆)haloalkyl or (C₃-C₆)cycloalkyl,
R³ is hydrogen, cyano, halogen, (C₃-C₆)cycloalkyl, (C₁-C₆)alkyl-(C₃-C₆)cycloalkyl, halo(C₃-C₆)cycloalkyl, cyano(C₃-C₆)cycloalkyl, (C₁-C₆)alkyl, (C₁—C₆)haloalkyl, (C₁-C₆)cyanoalkyl, (C₁-C₆)alkoxy-(C₁-C₆)alkyl, (C₂-C₆)alkenyl, (C₂-C₆)haloalkenyl, (C₂-C₆)cyanoalkenyl, (C₂-C₆)alkynyl, (C₂-C₆)haloalkynyl, (C₂-C₆)cyanoalkynyl, (C₁-C₆)alkoxy, (C₁-C₆)haloalkoxy, (C₁-C₆)alkoxyimino, (C₁-C₆)alkylthio, (C₁-C₆)haloalkylthio, (C₁-C₆)alkylsulfinyl, (C₁-C₆)haloalkylsulfinyl, (C₁-C₆)alkylsulfonyl, (C₁-C₆)haloalkylsulfonyl or (C₁-C₆)alkylsulfoximino,
R⁴ is hydrogen, (C₁-C₄)alkyl, (C₁-C₄), alkoxy-(C₁-C₄)alkyl or (C₁-C₄)haloalkyl,
R⁶ is hydrogen, cyano, halogen, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₃-C₆)cycloalkyl, (C₁-C₆)alkoxy, (C₁-C₆)haloalkoxy, (C₁-C₆)alkylthio, (C₁-C₆)haloalkylthio, (C₁-C₆)alkylsulfinyl, (C₁-C₆)haloalkylsulfinyl, (C₁-C₆)alkylsulfonyl, (C₁-C₆)haloalkylsulfonyl, (C₁-C₆)alkylcarbonyl or (C₁-C₆)haloalkylcarbonyl,
R⁷, R⁸ are independently hydrogen, cyano, halogen, (C₁-C₄)alkyl, (C₁-C₄)haloalkyl, (C₃-C₆)cycloalkyl, halo(C₃-C₆)cycloalkyl, cyano(C₃-C₆)cycloalkyl, (C₁-C₄)alkoxy, (C₁-C₄)haloalkoxy, (C₁-C₄)alkoxycarbonyl, (C₁-C₄)alkoxyimino, (C₁-C₄)alkylthio, (C₁-C₄)haloalkylthio, (C₁-C₄)alkylsulfinyl, (C₁-C₄)haloalkylsulfinyl, (C₁-C₄)alkylsulfonyl or (C₁-C₄)haloalkylsulfonyl,
n is 0, 1 or 2.

4. The compound of formula (I) according to claim 1, in which
A¹ is nitrogen,
A³ is nitrogen or =C(H)—,
X is oxygen,
R¹ is (C₁-C₄)alkyl, (C₁-C₄)haloalkyl or (C₃-C₄)cycloalkyl,
R³ is hydrogen, cyano, halogen, (C₁-C₄)alkyl, (C₁-C₄)haloalkyl, (C₁-C₄)alkoxy, (C₁-C₄)haloalkoxy, (C₁-C₄)alkylthio, (C₁-C₄)haloalkylthio, (C₁-C₄)alkylsulfinyl, (C₁—C₄)haloalkylsulfinyl, (C₁-C₄)alkylsulfonyl, (C₁-C₄)haloalkylsulfonyl or (C₁-C₄)alkoxyimino,
R⁴ is hydrogen or (C₁-C₄)alkyl,
R⁶ is hydrogen,
R⁷ is cyano, halogen, (C₁-C₄)alkyl, (C₁-C₄)haloalkyl, (C₃-C₄)cycloalkyl, cyano(C₃-C₄)cycloalkyl, (C₁-C₄)alkoxy, (C₁-C₄)haloalkoxy, (C₁-C₄)alkoxycarbonyl, (C₁-C₄)alkoxyimino, (C₁-C₄)alkylthio, (C₁-C₄)haloalkylthio, (C₁-C₄)alkylsulfinyl, (C₁-C₄)haloalkylsulfinyl, (C₁-C₄)alkylsulfonyl or (C₁-C₄)haloalkylsulfonyl,
R⁸ is hydrogen or cyano,
n is 0, 1 or 2.

5. The compound of formula (I) according to claim 1, in which
A¹ is nitrogen,
A³ is nitrogen or =C(H)—,
X is oxygen,
R¹ is methyl, ethyl, n-propyl or i-propyl,
R³ is hydrogen,
R⁴ is methyl,
R⁶ is hydrogen,
R⁷ is cyano, fluorine, chlorine, bromine, iodine, methyl, ethyl, trifluoromethyl, methoxy, trifluoromethoxy, methoxycarbonyl, methoxyimino or cyanocyclopropyl,
R⁸ is hydrogen or chlorine,
n is 0, 1 or 2.

6. The compound of formula (I) according to claim 1, in which
A¹ is nitrogen,
A³ is nitrogen or =C(H)—,
X is oxygen,
R¹ is ethyl,
R³ is hydrogen,
R⁴ is methyl,
R⁶ is hydrogen,
R⁷ is cyano, fluorine, chlorine, bromine, iodine, methoxycarbonyl (—COOCH₃), methoxyimino (—CH═NOCH₃) or 1-cyano-1-cyclopropyl,
R⁸ is hydrogen or chlorine,
n is 2.

7. The compound of formula (I) according to claim 1, in which A³ is nitrogen and A¹, X, R¹, R³, R⁴, R⁶, R⁷, R⁸ and n have the definitions given in claim 1, 2, 3, 4, 5 or 6.

8. The compound of formula (I) according to claim 1, in which A³ is =C(H)— and A¹, X, R¹, R³, R⁴, R⁶, R⁷, R⁸ and n have the definitions given in claim 1, 2, 3, 4, 5 or 6.

9. The compound of formula (I) according to claim 1, in which the compounds have the following structure:

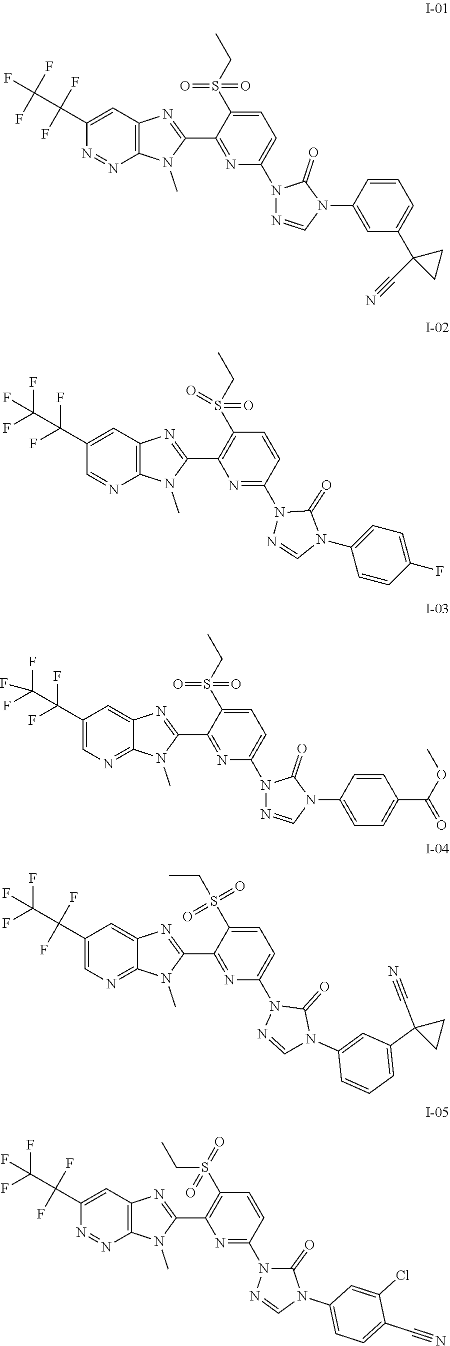 [SMILES: CCS(=O)(=O)c1ccc(-n2ncn(-c3cccc(C4(C#N)CC4)c3)c2=O)nc1-c1nc2cc(C(F)(F)C(F)(F)F)nnc2n1C ; CCS(=O)(=O)c1ccc(-n2ncn(-c3ccc(F)cc3)c2=O)nc1-c1nc2cc(C(F)(F)C(F)(F)F)cnc2n1C ; CCS(=O)(=O)c1ccc(-n2ncn(-c3ccc(C(=O)OC)cc3)c2=O)nc1-c1nc2cc(C(F)(F)C(F)(F)F)cnc2n1C ; CCS(=O)(=O)c1ccc(-n2ncn(-c3cccc(C4(C#N)CC4)c3)c2=O)nc1-c1nc2cc(C(F)(F)C(F)(F)F)cnc2n1C ; CCS(=O)(=O)c1ccc(-n2ncn(-c3ccc(C#N)c(Cl)c3)c2=O)nc1-c1nc2cc(C(F)(F)C(F)(F)F)nnc2n1C]

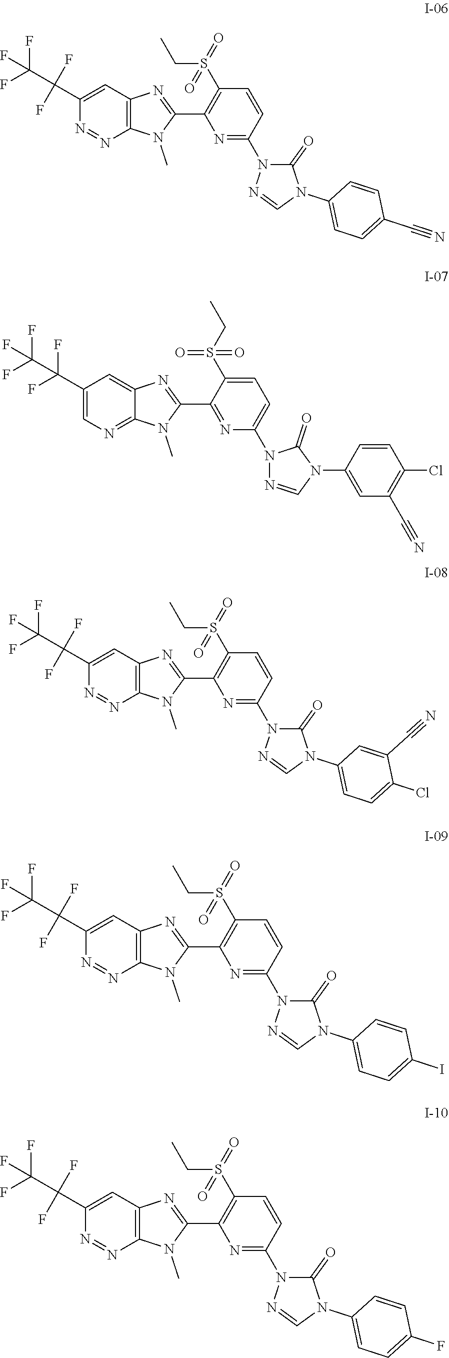 [SMILES: CCS(=O)(=O)c1ccc(-n2ncn(-c3ccc(C#N)cc3)c2=O)nc1-c1nc2cc(C(F)(F)C(F)(F)F)nnc2n1C ; CCS(=O)(=O)c1ccc(-n2ncn(-c3ccc(Cl)c(C#N)c3)c2=O)nc1-c1nc2cc(C(F)(F)C(F)(F)F)cnc2n1C ; CCS(=O)(=O)c1ccc(-n2ncn(-c3ccc(Cl)c(C#N)c3)c2=O)nc1-c1nc2cc(C(F)(F)C(F)(F)F)nnc2n1C ; CCS(=O)(=O)c1ccc(-n2ncn(-c3ccc(I)cc3)c2=O)nc1-c1nc2cc(C(F)(F)C(F)(F)F)nnc2n1C ; CCS(=O)(=O)c1ccc(-n2ncn(-c3ccc(F)cc3)c2=O)nc1-c1nc2cc(C(F)(F)C(F)(F)F)nnc2n1C]

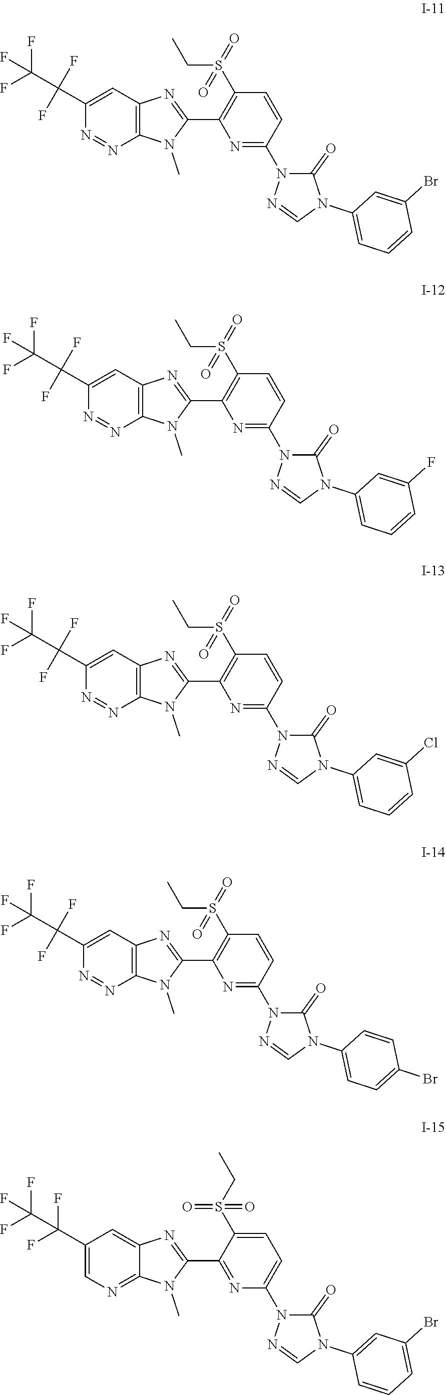 [SMILES: CCS(=O)(=O)c1ccc(-n2ncn(-c3cccc(Br)c3)c2=O)nc1-c1nc2cc(C(F)(F)C(F)(F)F)nnc2n1C ; CCS(=O)(=O)c1ccc(-n2ncn(-c3cccc(F)c3)c2=O)nc1-c1nc2cc(C(F)(F)C(F)(F)F)nnc2n1C ; CCS(=O)(=O)c1ccc(-n2ncn(-c3cccc(Cl)c3)c2=O)nc1-c1nc2cc(C(F)(F)C(F)(F)F)nnc2n1C ; CCS(=O)(=O)c1ccc(-n2ncn(-c3ccc(Br)cc3)c2=O)nc1-c1nc2cc(C(F)(F)C(F)(F)F)nnc2n1C ; CCS(=O)(=O)c1ccc(-n2ncn(-c3cccc(Br)c3)c2=O)nc1-c1nc2cc(C(F)(F)C(F)(F)F)cnc2n1C]

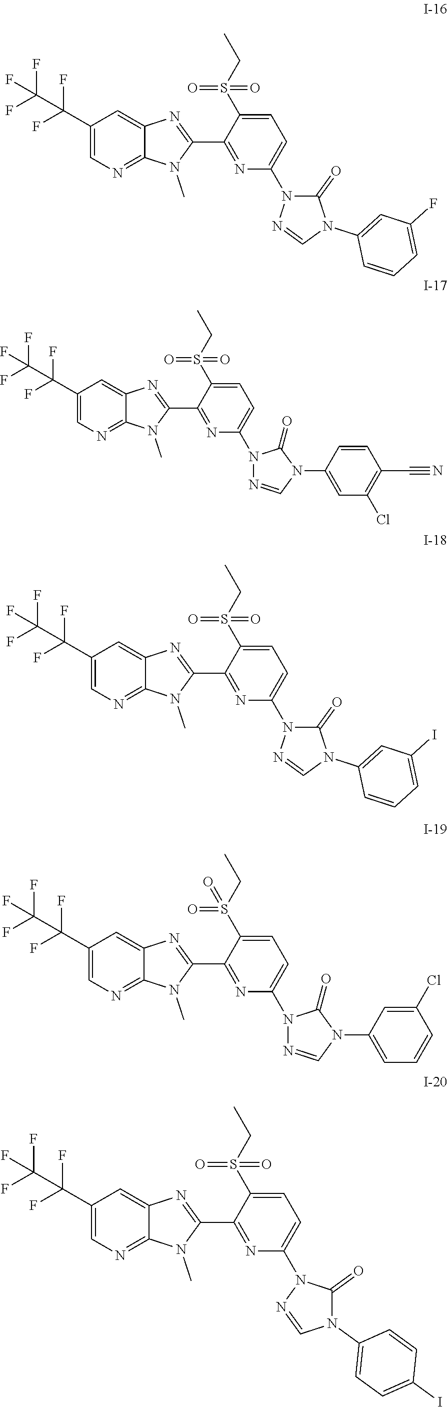 [SMILES: CCS(=O)(=O)c1ccc(-n2ncn(-c3cccc(F)c3)c2=O)nc1-c1nc2cc(C(F)(F)C(F)(F)F)cnc2n1C ; CCS(=O)(=O)c1ccc(-n2ncn(-c3ccc(C#N)c(Cl)c3)c2=O)nc1-c1nc2cc(C(F)(F)C(F)(F)F)cnc2n1C ; CCS(=O)(=O)c1ccc(-n2ncn(-c3cccc(I)c3)c2=O)nc1-c1nc2cc(C(F)(F)C(F)(F)F)cnc2n1C ; CCS(=O)(=O)c1ccc(-n2ncn(-c3cccc(Cl)c3)c2=O)nc1-c1nc2cc(C(F)(F)C(F)(F)F)cnc2n1C ; CCS(=O)(=O)c1ccc(-n2ncn(-c3ccc(I)cc3)c2=O)nc1-c1nc2cc(C(F)(F)C(F)(F)F)cnc2n1C]

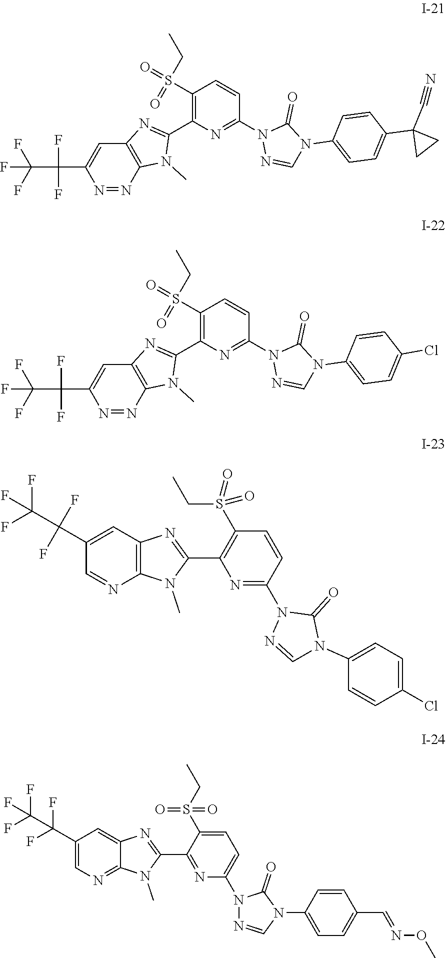 [SMILES: CCS(=O)(=O)c1ccc(-n2ncn(-c3ccc(C4(C#N)CC4)cc3)c2=O)nc1-c1nc2cc(C(F)(F)C(F)(F)F)nnc2n1C ; CCS(=O)(=O)c1ccc(-n2ncn(-c3ccc(Cl)cc3)c2=O)nc1-c1nc2cc(C(F)(F)C(F)(F)F)nnc2n1C ; CCS(=O)(=O)c1ccc(-n2ncn(-c3ccc(Cl)cc3)c2=O)nc1-c1nc2cc(C(F)(F)C(F)(F)F)cnc2n1C ; CCS(=O)(=O)c1ccc(-n2ncn(-c3ccc(/C=N/OC)cc3)c2=O)nc1-c1nc2cc(C(F)(F)C(F)(F)F)cnc2n1C]

10. An Agrochemical formulation comprising a compound of formula (I) according to claim 1, and one or more extenders and/or surfactants.

11. The Agrochemical formulation according to claim 10, additionally comprising a further active agrochemical ingredient.

12. A method of controlling animal pests, comprising allowing a compound according to claim 1 to act on one or more animal pests and/or a habitat thereof.

13. A compound according to claim 1 or an agrochemical formulation thereof for controlling one or more animal pests.
